SEQUENCE LISTING (122 sequences)

SEQ ID NO: 1 - protein, 6 aa, synthetic construct
FSSSSI

SEQ ID NO: 2 - protein, 10 aa, synthetic construct
SISSSYGYTY

SEQ ID NO: 3 - protein, 4 aa, synthetic construct
SWAM

SEQ ID NO: 4 - protein, 5 aa, synthetic construct
SVSSA

SEQ ID NO: 5 - protein, 7 aa, synthetic construct
SASSLYS

SEQ ID NO: 6 - protein, 8 aa, synthetic construct
YWAYYSPI

SEQ ID NO: 7 - protein, 7 aa, synthetic construct
DFTAYAM

SEQ ID NO: 8 - protein, 10 aa, synthetic construct
SIYPSGGYTA

SEQ ID NO: 9 - protein, 7 aa, synthetic construct
RSYYFAL

SEQ ID NO: 10 - protein, 5 aa, synthetic construct
SVSSA

SEQ ID NO: 11 - protein, 7 aa, synthetic construct
SASDLYS

SEQ ID NO: 12 - protein, 7 aa, synthetic construct
YAGAGLI

SEQ ID NO: 13 - protein, 115 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSS

SEQ ID NO: 14 - protein, 109 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIK

SEQ ID NO: 15 - protein, 118 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSS

SEQ ID NO: 16 - protein, 108 aa, synthetic construct
DIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIK

SEQ ID NO: 17 - protein, 107 aa, synthetic construct
LLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 18 - protein, 107 aa, synthetic construct
AAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 19 - protein, 107 aa, synthetic construct
AAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAK

SEQ ID NO: 20 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 107 aa, synthetic construct
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 107 aa, synthetic construct
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 107 aa, synthetic construct
GQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 107 aa, synthetic construct
GQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 228 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIK

SEQ ID NO: 26 - protein, 232 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIK

SEQ ID NO: 27 - protein, 5 aa, synthetic construct
DKTHT

SEQ ID NO: 28 - protein, 13 aa, synthetic construct
DKTHTCPPCPAPE

SEQ ID NO: 29 - protein, 5 aa, synthetic construct
GGGGS

SEQ ID NO: 30 - protein, 9 aa, synthetic construct
GGGGSGGGS

SEQ ID NO: 31 - protein, 15 aa, synthetic construct
GGGGSGGGGSEPKSC

SEQ ID NO: 32 - protein, 15 aa, synthetic construct
GGGGSGGGGSEPKSS

SEQ ID NO: 33 - protein, 20 aa, synthetic construct
GGGGSGGGGSEPKSSDKTHT

SEQ ID NO: 34 - protein, 20 aa, synthetic construct
GGGSGGGSGGGSGGGSGSTG

SEQ ID NO: 35 - protein, 6 aa, synthetic construct
LSSYSM

SEQ ID NO: 36 - protein, 10 aa, synthetic construct
YISSYDSITD

SEQ ID NO: 37 - protein, 8 aa, synthetic construct
PAVGHMAF

SEQ ID NO: 38 - protein, 5 aa, synthetic construct
SVSSA

SEQ ID NO: 39 - protein, 7 aa, synthetic construct
SASSLYS

SEQ ID NO: 40 - protein, 6 aa, synthetic construct
WYNAPI

SEQ ID NO: 41 - protein, 119 aa, synthetic construct
EVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSS

SEQ ID NO: 42 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIK

SEQ ID NO: 43 - protein, 107 aa, synthetic construct [sequence omitted]

SEQ ID NO: 44 - protein, 214 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 108 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 47 - protein, 102 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTII

SEQ ID NO: 48 - protein, 103 aa, synthetic construct
DVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 49 - protein, 205 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 50 - protein, 5 aa, synthetic construct
SYSMG

SEQ ID NO: 51 - protein, 16 aa, synthetic construct
AISKGGYKYDSVSLEG

SEQ ID NO: 52 - protein, 17 aa, synthetic construct
SRAYGSSRLRLADTYEY

SEQ ID NO: 53 - protein, 125 aa, synthetic construct
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 54 - skipped

SEQ ID NO: 55 - protein, 16 aa, synthetic construct
RISSGGTTAYVDSVKG

SEQ ID NO: 56 - protein, 7 aa, synthetic construct
FSSRPNP

SEQ ID NO: 57 - protein, 113 aa, synthetic construct
EVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 58 - protein, 15 aa, synthetic construct
MNLLLILTFVAAAVA

SEQ ID NO: 59 - protein, 717 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGSGGGSGGGSGGGSGSTGEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 60 - protein, 721 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSGSTGEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 61 - protein, 214 aa, synthetic construct; identical to SEQ ID NO: 44

SEQ ID NO: 62 - protein, 717 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 63 - protein, 721 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 64 - protein, 348 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 65 - protein, 851 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 66 - protein, 855 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 67 - protein, 214 aa, synthetic construct; identical to SEQ ID NO: 44

SEQ ID NO: 68 - protein, 717 aa, synthetic construct; identical to SEQ ID NO: 62

SEQ ID NO: 69 - protein, 721 aa, synthetic construct; identical to SEQ ID NO: 63

SEQ ID NO: 70 - protein, 470 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 71 - protein, 973 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 72 - protein, 977 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 73 - protein, 214 aa, synthetic construct; identical to SEQ ID NO: 44

SEQ ID NO: 74 - protein, 851 aa, synthetic construct; identical to SEQ ID NO: 65

SEQ ID NO: 75 - protein, 855 aa, synthetic construct; identical to SEQ ID NO: 66

SEQ ID NO: 76 - protein, 348 aa, synthetic construct; identical to SEQ ID NO: 64

SEQ ID NO: 77 - protein, 973 aa, synthetic construct; identical to SEQ ID NO: 71

SEQ ID NO: 78 - protein, 977 aa, synthetic construct; identical to SEQ ID NO: 72

SEQ ID NO: 79 - protein, 470 aa, synthetic construct; identical to SEQ ID NO: 70

SEQ ID NO: 80 - protein, 434 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 81 - protein, 439 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEPKSSDKTHTSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 82 - protein, 247 aa, synthetic construct
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 83 - protein, 247 aa, synthetic construct
EVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSSGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 84 - protein, 19 aa, synthetic construct
MGWSCIILFLVATATGVHS

SEQ ID NO: 85 - protein, 20 aa, synthetic construct
METDTLLLWVLLLWVPGSTG

SEQ ID NO: 86 - protein, 19 aa, synthetic construct
MEWSWVFLFFLSVTTGVHS

SEQ ID NO: 87 - protein, 942 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGAGGGGAGGGGAGGGGASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 88 - protein, 946 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGAGGGGAGGGGAGGGGASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 89 - protein, 214 aa, synthetic construct; identical to SEQ ID NO: 44

SEQ ID NO: 90 - protein, 942 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGAGGGGAGGGGAGGGGAEVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 91 - protein, 946 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGAGGGGAGGGGAGGGGAEVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 92 - protein, 214 aa, synthetic construct; identical to SEQ ID NO: 44

SEQ ID NO: 93 - protein, 942 aa, synthetic construct; identical to SEQ ID NO: 87

SEQ ID NO: 94 - protein, 946 aa, synthetic construct; identical to SEQ ID NO: 88

SEQ ID NO: 95 - protein, 439 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGAGGGGAGGGGAGGGGASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 96 - protein, 942 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 97 - protein, 946 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 98 - protein, 214 aa, synthetic construct; identical to SEQ ID NO: 44

SEQ ID NO: 99 - protein, 942 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSGGGGSEVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 100 - protein, 946 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSGGGGSEVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 101 - protein, 214 aa, synthetic construct; identical to SEQ ID NO: 44

SEQ ID NO: 102 - protein, 942 aa, synthetic construct; identical to SEQ ID NO: 96

SEQ ID NO: 103 - protein, 946 aa, synthetic construct; identical to SEQ ID NO: 97

SEQ ID NO: 104 - protein, 439 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQWYNAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 105 - protein, 932 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 106 - protein, 936 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSEVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 107 - protein, 214 aa, synthetic construct; identical to SEQ ID NO: 44

SEQ ID NO: 108 - protein, 937 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 109 - protein, 941 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSEVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 110 - protein, 214 aa, synthetic construct; identical to SEQ ID NO: 44

SEQ ID NO: 111 - protein, 932 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGAGGGGAEVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 112 - protein, 936 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGAGGGGAEVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 113 - protein, 214 aa, synthetic construct; identical to SEQ ID NO: 44

SEQ ID NO: 114 - protein, 717 aa, synthetic construct
EVQLVESGGGLVQPGGSLRLSCAASGFDFSSSSIHWVRQAPGKGLEWVASISSSYGYTYYADSVKGRFTISADTSKNTAYLQMNSLRAEDTAVYYCARSWAMDYWGQGTLVTVSSGGGGSDIQMTQSPSSLSASVGDRVTITCRASQSVSSAVAWYQQKPGKAPKLLIYSASDLYSGVPSRFSGSRSGTDFTLTISSLQPEDFATYYCQQYAGAGLITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPIRELMTSNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 115 - protein, 721 aa, synthetic construct
EVQLVESGGGLVQPGGSLRLSCAASGFDFTAYAMHWVRQAPGKGLEWVASIYPSGGYTAYADSVKGRFTISADTSKNTAYLQMNSLRAEDTAVYYCARRSYYFALDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWAYYSPITFGQGTKVEIKGGGGSGGGGSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPMVFDLPPSREEMTKNQVSLWCMVKGFYPSDIAVEWESNGQPENNYKTTPPVLDSDGSFFLYSKLTVDKSRWQQGNVFSCSVMHEALHNHYTQKSLSLSPGKGGGGSGGGGSEPKSSDKTHTEVQLVESGGGLVQPGGSLRLSCAASGFTLSSYSMHWVRQAPGKGLEWVAYISSYDSITDYADSVKGRFTISADTSKNTAYLQMNSLRAEDTAVYYCARPAVGHMAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 116 - protein, 439 aa, synthetic construct; identical to SEQ ID NO: 104

SEQ ID NO: 117 - protein, 5 aa, synthetic construct
GGGGA

SEQ ID NO: 118 - protein, 20 aa, synthetic construct
GGGGAGGGGAGGGGAGGGGA

SEQ ID NO: 119 - protein, 15 aa, synthetic construct
GGGGAGGGGAGGGGA

SEQ ID NO: 120 - protein, 10 aa, synthetic construct
GGGGSGGGGS

SEQ ID NO: 121 - protein, 15 aa, synthetic construct
GGGGSGGGGSGGGGS

SEQ ID NO: 122 - protein, 20 aa, synthetic construct
GGGGSGGGGSGGGGSGGGGS

(19) United States
(12) Patent Application Publication
(10) Pub. No.: US 2026/0092109 A1
(43) Pub. Date: Apr. 2, 2026

(54) MULTIVALENT FZD4, WNT CO-RECEPTOR, AND VEGF RECEPTOR MOLECULES AND USES THEREOF

(71) Applicants: EyeBiotech Limited, London (GB); Merck Sharp & Dohme LLC, Rahway, NJ (US)

(72) Inventors: Paul E. Stephens, Wiltshire (GB); Michael John Davies, Middlesex (GB); Yin Shan Eric Ng, Groton, MA (US); Masahisa Handa, Berkeley, CA (US); Alexander V. Loktev, Danville, CA (US)

(73) Assignees: EyeBiotech Limited, London (GB); Merck Sharp & Dohme LLC, Rahway, NJ (US)

(21) Appl. No.: 19/319,282

(22) Filed: Sep. 4, 2025

Related U.S. Application Data
(60) Provisional application No. 63/691,710, filed on Sep. 6, 2024.

(51) Int. Cl. C07K 16/28; C07K 16/22

(52) U.S. Cl. CPC C07K 16/28; C07K 16/22; C07K 16/2863; C07K 2317/31; C07K 2317/52; C07K 2317/76; C07K 2317/92

(57) ABSTRACT

Herein are multivalent antibody binding molecules that activate a Wnt/β-catenin signaling pathway comprising a FZD4 receptor binding domain, an LRP5 co-receptor binding domain, and a VEGF binding domain. Also described herein are nucleic acids and vectors encoding said molecules and methods for their use.

### CROSS-REFERENCE TO RELATED APPLICATIONS

The application claims the benefit of priority to U.S. Provisional Application No. 63/691,710, filed Sep. 6, 2024, the contents of which are incorporated herein by reference in their entirety.

### REFERENCE TO SEQUENCE LISTING SUBMITTED ELECTRONICALLY

The contents of the electronic sequence listing (26044-US-PCT_SL.xml; Size: 145,065 bytes, created on Sep. 2, 2025) are herein incorporated by reference in their entirety

### FIELD

This disclosure relates generally to multivalent antibody binding molecules that activate a Wnt/β-catenin signaling pathway comprising a FZD4 receptor binding domain, an LRP5 co-receptor binding domain, and a VEGF binding domain. Also described herein are nucleic acids and vectors encoding said molecules and methods for their use.

### BACKGROUND

Wnt signaling pathways are critical for embryonic development and tissue homeostasis in adults. Wnt signaling is initiated when a Frizzled (FZD) receptor on the cell surface membrane binds with a Wnt ligand. Wnt ligands are secreted growth factors that regulate various cellular processes such as proliferation, differentiation, survival and migration.

Nineteen Wnt ligands exist in humans that interact with a network of ten Frizzled cell surface receptors (FZD) and one of several co-receptors that guide the selective engagement of different intracellular signaling branches (Wodarz, A. and Nusse, R. Annu. Rev. Cell Dev. Biol. 14, 59-88 (1998); Angers, S and Moon, R. T., transduction. Nat. Rev. Mol. Cell Biol. 10, 468-477 (2009)). FZDs have conserved structural features including seven hydrophobic transmembrane domains and a cysteine-rich ligand-binding domain. FZDs are known to function in three distinct signaling pathways, known as the Wnt planar cell polarity (PCP) pathway, the canonical Wnt/β-catenin pathway, and the Wnt/calcium pathway. The presence of Wnt co-receptors is also required to direct the differential engagement of the intracellular signaling cascades listed above. For example, Wnt ligands bind to a Frizzled receptor and a member of the low-density lipoprotein receptor-related proteins 5 and 6 (LRP5/6) co-receptor family to activate the Wnt/β-catenin pathway, or with a receptor tyrosine kinase-like orphan receptors 1 and 2 (ROR1/2), related to receptor tyrosine kinase (RYK) or protein tyrosine kinase 7 (PTK7) co-receptor to activate alternate β-catenin-independent signaling pathways. Wnt ligands are universally important for the control of tissue stem cells self-renewal and regulation of many progenitor cell populations, but the hydrophobicity and sensitive tertiary structure of Wnt proteins makes their biochemical purification challenging and their use in vitro and in vivo inefficient.

### SUMMARY

The present disclosure is directed to a multivalent antibody binding molecule, comprising an LRP5 binding domain that specifically binds low-density lipoprotein receptor-related protein 5 (LRP5), an Fc domain, an FZD4 binding domain that specifically binds Frizzled 4 (FZD4), and one or more VEGF binding domains that each specifically bind vascular endothelial growth factor (VEGF); wherein the multivalent antibody molecule comprises four polypeptides, wherein: (a) the first polypeptide comprises: (i) a first VH comprising a CDR-H1 having the amino acid sequence of SEQ ID NO: 1, a CDR-H2 having the amino acid sequence of SEQ ID NO: 2, and a CDR-H3 having the amino acid sequence of SEQ ID NO: 3; (ii) a VL comprising a CDR-L1 having the amino acid sequence of SEQ ID NO: 10, a CDR-L2 having the amino acid sequence of SEQ ID NO: 11, and a CDR-L3 having the amino acid sequence of SEQ ID NO: 12; and (iii) a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37; (b) the second polypeptide comprises: (i) a first VH comprising a CDR-H1 having the amino acid sequence of SEQ ID NO: 7, a CDR-H2 having the amino acid sequence of SEQ ID NO: 8, and a CDR-H3 having the amino acid sequence of SEQ ID NO: 9; (ii) a VL comprising a CDR-L1 having the amino acid sequence of SEQ ID NO: 4, a CDR-L2 having the amino acid sequence of SEQ ID NO: 5, and a CDR-L3 having the amino acid sequence of SEQ ID NO: 6; and (iii) a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37; and (c) the third and fourth polypeptides comprise a VL comprising a CDR-L1 having the amino acid sequence of SEQ ID NO: 38, a CDR-L2 having the amino acid sequence of SEQ ID NO: 39, and a CDR-L3 having the amino acid sequence of SEQ ID NO: 40, and wherein each of the VEGF binding domains comprise a VEGF receptor component. In some aspects, the LRP5 binding domain is attached to the N-terminus of the Fc domain and the FZD4 binding domain is attached to the C-terminus the Fc domain, wherein the LRP5 binding domain comprises a diabody that binds LRP5, and wherein the FZD4 binding domain comprises two scFv molecules or two Fab molecules that bind FZD4. The present disclosure is directed to a multivalent antibody binding molecule, comprising an LRP5 binding domain that specifically binds low-density lipoprotein receptor-related protein 5 (LRP5), an Fc domain, an FZD4 binding domain that specifically binds Frizzled 4 (FZD4), and one or more VEGF binding domains that each specifically bind vascular endothelial growth factor (VEGF); wherein: (a) the LRP5 binding domain is attached to the N-terminus of the Fc domain and the FZD4 binding domain is attached to the C-terminus the Fc domain, (b) the LRP5 binding domain comprises a diabody that binds LRP5, (c) the FZD4 binding domain comprises two scFv molecules or two Fab molecules that bind FZD4, and (d) each of the VEGF binding domains comprise one or more single heavy chain variable domain antibodies (VHH) or antigen binding fragments thereof, wherein each VHH or antigen binding fragment comprises: (i) a heavy chain complementarity determining region 1 (HC-CDR1) comprising the amino acid sequence set forth in SEQ ID NO: 50; an HC-CDR2 comprising the amino acid sequence set forth in SEQ ID NO: 51; and an HC-CDR3 comprising the amino acid sequence set forth in SEQ ID NO: 52, or (ii) an HC-CDR1 comprising the amino acid set forth in SEQ ID NO: 54, an HC-CDR2 comprising the amino acid sequence set forth in SEQ ID NO: 55 and the HC-CDR3 comprising the amino acid sequence set forth in SEQ ID NO: 56. In some aspects, each VHH comprises an HC-CDR1 comprising the amino acid sequence set forth in SEQ ID NO: 50; an HC-CDR2 comprising the amino acid sequence set forth in SEQ ID NO: 51; and an HC-CDR3 comprising the amino acid sequence set forth in SEQ ID NO: 52. In some aspects, each VHH comprises an HC-CDR1 comprising the amino acid set forth in SEQ ID NO:54, an HC-CDR2 comprising the amino acid sequence set forth in SEQ ID NO:55 and the HC-CDR3 comprising the amino acid sequence set forth in SEQ ID NO:56.

In some aspects, each of the VEGF binding domains comprise one or more VHH. In some aspects, each VHH comprises an amino acid sequence that is at least 90% identical to SEQ ID NO: 53 or SEQ ID NO: 57. In some aspects, each VHH comprises an amino acid sequence that is at least 95% identical to SEQ ID NO: 53 or SEQ ID NO: 57. In some aspects, each VHH comprises an amino acid sequence that is at least 90% identical to SEQ ID NO: 53. In some aspects, each VHH comprises an amino acid sequence that is at least 95% identical to SEQ ID NO: 57. In some aspects, each VHH comprises an amino acid sequence according to SEQ ID NO: 53 or SEQ ID NO: 57. In some aspects, each VHH comprises an amino acid sequence according to SEQ ID NO: 53. In some aspects, each VHH comprises an amino acid sequence according to SEQ ID NO: 57. In some aspects, the multivalent antibody binding molecule comprises a first VEGF binding domain that is attached to the C-terminus of one or both of the scFv molecules or the Fab molecules.

In some aspects, the FZD4 binding domain comprises two Fab molecules that bind FZD4. In some aspects, the multivalent antibody binding molecule comprises a first VEGF binding domain that is attached to the C-terminus of one or both of the scFv molecules or the Fab molecules. In some aspects, the FZD4 binding domain comprises two Fab molecules that bind FZD4, and wherein the multivalent antibody binding molecule comprises a first VEGF binding domain and second VEGF binding domain, wherein each VEGF binding domain is attached to the C-terminus of each of the Fab molecules. In some aspects, the first VEGF binding domain is attached to the C-terminus of the third polypeptide, and the second VEGF binding domain is attached to the C-terminus of the fourth polypeptide.

In some aspects, the FZD4 binding domain comprises two Fab molecules that bind FZD4, and wherein the multivalent antibody binding molecule comprises a first VEGF binding domain and second VEGF binding domain, wherein each VEGF binding domain is attached to the N-terminus of the diabody that binds LRP5. In some aspects, the first VEGF binding domain and/or the second VEGF binding domain comprises a VEGF receptor 1 (VEGFR1) extracellular Ig domain 2 and a VEGF receptor 2 (VEGFR2) extracellular Ig domain number 3. In some aspects, the first VEGF binding domain and/or the second VEGF binding domain comprises an amino acid sequence at least 90% identical to a sequence selected from SEQ ID NO: 47-49.

In some aspects, the first VEGF binding domain and/or the second VEGF binding domain comprises an amino acid sequence at least 95% identical to a sequence selected from SEQ ID NO: 47-49. In some aspects, the first VEGF binding domain and/or the second VEGF binding domain comprises an amino acid sequence at least 90% identical to SEQ ID NO: 49.

In some aspects, the first VEGF binding domain and/or the second VEGF binding domain comprises an amino acid sequence at least 95% identical to SEQ ID NO: 49. In some aspects, the first VEGF binding domain and the second VEGF binding domain each comprise the amino acid sequence of SEQ ID NO: 49. In some aspects, the first VH of the first polypeptide interacts with the VL of the second polypeptide to form a domain that binds LRP5; the first VH of the second polypeptide interacts with the VL of the first polypeptide to form a domain that binds LRP5; the second VH of the first polypeptide interacts with the VL of the third polypeptide to form a domain that binds FZD4; and the second VH of the second polypeptide interacts with the VL of the fourth polypeptide to form a domain that binds FZD4.

In some aspects, (a) the first polypeptide comprises: (i) a first VH comprising an amino acid sequence that is 90% identical to SEQ ID NO: 13, (ii) a VL comprising an amino acid sequence that is 90% identical to SEQ ID NO: 16; and (iii) a second VH comprising an amino acid sequence that is 90% identical to SEQ ID NO: 41; (b) the second polypeptide comprises: (iv) a first VH comprising an amino acid sequence that is 90% identical to SEQ ID NO: 15; (v) a VL comprising an amino acid sequence that is 90% identical to SEQ ID NO: 14; and (vi) a second VH comprising an amino acid sequence that is 90% identical to SEQ ID NO: 41; and (c) the third polypeptide and the fourth polypeptide comprise a VL comprising an amino acid sequence that is 90% identical to SEQ ID NO: 42.

In some aspects, each VEGF binding domain comprises one VHH. In some aspects, each VEGF binding domain comprises two VHH molecules. In some aspects, the multivalent antibody binding molecule comprises a first VEGF binding domain and a second VEGF binding domain, wherein the first VEGF binding domain and/or the second VEGF binding domain comprise one VHH. In some aspects, the multivalent antibody binding molecule comprises a first VEGF binding domain and a second VEGF binding domain, wherein the first VEGF binding domain and the second VEGF binding domain each comprise two VHHs. In some aspects, the third VEGF binding domain and the fourth VEGF binding domain each comprise one VHH. In some aspects, the third VEGF binding domain and the fourth VEGF binding domain each comprise two VHHs. In some aspects, the VHHs are separated by a VHH linker. In some aspects, the VHH linker comprises one or more of the amino acid sequence of SEQ ID NO: 29. In some aspects, the multivalent antibody binding molecule comprises a first VEGF binding domain, a second VEGF binding domain, a third VEGF binding domain, and a fourth VEGF binding domain, each comprising two VHHs separated by a VHH linker and comprising the sequence according to SEQ ID NO: 82 or SEQ ID NO: 83.

In some aspects, the first polypeptide and the second polypeptide form a heterodimer and each comprise a constant heavy chain domain 2 (CH2) and a constant heavy chain domain 3 (CH3), wherein the CH2 and CH3 of the first polypeptide and the CH2 and CH3 of the second polypeptide form the Fc domain. In some aspects, the CH3 of the first polypeptide comprises a serine at position 366, an alanine at position 368 and a valine at position 407 and the CH3 of the second polypeptide comprises a tryptophan at position 366; or the CH3 of the second polypeptide comprises a serine at position 366, an alanine at position 368 and a valine at position 407, and the CH3 of the first polypeptide comprises a tryptophan at position 366, and wherein the positions are according to EU numbering.

In some aspects, the CH3 of the first polypeptide further comprises an isoleucine at position 354, a leucine at position 357, and a serine at position 360, and the CH3 of the second polypeptide further comprises a methionine at position 347, a phenylalanine at position 349, an aspartic acid at position 350, and a methionine a position 368; or the CH3 of the second polypeptide further comprises an isoleucine at position 354, a leucine at position 357, and a serine at position 360, and the CH3 of the first polypeptide further comprises a methionine at position 347, a phenylalanine at position 349, an aspartic acid at position 350, and a methionine a position 368; and wherein the positions are according to EU numbering.

In some aspects, the first polypeptide comprises a cysteine at position 349, and the second polypeptide comprises a cysteine at position 354, or the second polypeptide comprises a cysteine at position 349, and the first polypeptide comprises a cysteine at position 354, and wherein the positions are according to EU numbering. In some aspects, the Fc domain has reduced effector function. In some aspects, the CH3 of the first polypeptide and/or the CH3 of the second polypeptide comprise a glycine at position 397, an alanine at position 265, or both a glycine at position 397 and an alanine at position 265, wherein the positions according to EU numbering. In some aspects, the CH3 of the first polypeptide and/or the CH3 of the second polypeptide comprise a glycine at position 397 and an alanine at position 265, wherein the positions according to EU numbering. In some aspects, the CH2 of the first polypeptide and/or the CH2 of the second polypeptide comprise an alanine at position 234, an alanine at position 235, a serine at position 331, or any combination thereof, wherein the positions are according to EU numbering. In some aspects, the CH2 of the first polypeptide and/or the CH2 of the second polypeptide comprise an alanine at position 234 and an alanine at position 235, wherein the positions are according to EU numbering. In some aspects, the CH2 of the first polypeptide and the CH2 of the second polypeptide comprise an alanine at position 234, an alanine at position 235, and a serine a position 331, wherein the positions are according to EU numbering. In some aspects, the CH2 of the first polypeptide and the CH2 of the second polypeptide comprise an alanine at position 234, an alanine at position 235, and a serine a position 265, wherein the positions are according to EU numbering.

In some aspects, the VL of the first polypeptide is attached to the CH2 of the first polypeptide by a first polypeptide linker, and the VL of the second polypeptide is attached to CH2 of the second polypeptide by a second polypeptide linker. In some aspects, the second VH of the first polypeptide is attached to the CH3 of the first polypeptide by a third polypeptide linker, and the second VH of the second polypeptide is attached to CH3 of the second polypeptide by a fourth polypeptide linker. In some aspects, the first polypeptide linker and/or the second polypeptide linker comprise the amino acid sequence selected from SEQ ID NOs: 27-34 or SEQ ID NOs: 117-119. In some aspects, the first polypeptide linker and/or the second polypeptide linker comprise one or more repeats of the amino acid sequence of SEQ ID NO: 29 or SEQ ID NO: 117. In some aspects, the third polypeptide linker and/or the fourth polypeptide linker comprise the amino acid sequence selected from SEQ ID NOs: 27-34 or SEQ ID NOS: 117-119. In some aspects, the third polypeptide linker and/or the fourth polypeptide linker comprise one or more repeats of the amino acid sequence of SEQ ID NO: 29 or SEQ ID NO: 117. In some aspects, the first polypeptide linker and/or the second polypeptide linker comprise the amino acid sequence of GGGGSGGGGSEPKSS (SEQ ID NO: 32). In some aspects, the first polypeptide linker and/or the second polypeptide linker comprise the amino acid sequence of GGGGSGGGGSEPKSSDKTHT (SEQ ID NO: 33). In some aspects, the first polypeptide linker and the second polypeptide linker comprise the amino acid sequence of GGGGSGGGGSEPKSSDKTHT (SEQ ID NO: 33). In some aspects, the third polypeptide linker and/or the fourth polypeptide linker comprise the amino acid sequence of GGGSGGGSGGGSGGGSGSTG (SEQ ID NO: 34). In some aspects, the third polypeptide linker and the fourth polypeptide linker comprise the amino acid sequence of GGGSGGGSGGGSGGGSGSTG (SEQ ID NO: 34). In some aspects, the first VH of the first polypeptide is linked to the VL of the first polypeptide by a fifth polypeptide linker, and the first VH of the second polypeptide is linked to the VL of the second polypeptide by a sixth polypeptide linker. In some aspects, the fifth polypeptide linker comprises one or more repeats of the amino acid sequence of SEQ ID NO: 29 or SEQ ID NO: 117, and/or the sixth polypeptide linker comprises one or more repeats of the amino acid sequence of SEQ ID NO: 29 or SEQ ID NO: 117. In some aspects, the fifth polypeptide linker comprises the amino acid sequence of GGGGS (SEQ ID NO: 29); and the sixth polypeptide linker comprises the amino acid sequence of GGGGS (SEQ ID NO: 29). In some aspects, the first VEGF binding domain is attached to the second VH of the first polypeptide by a seventh polypeptide linker, and the second VEGF binding domain is attached to the second VH of the second polypeptide by an eighth polypeptide linker.

In some aspects, the seventh polypeptide linker and/or the eighth polypeptide linker comprise an amino acid sequence selected from SEQ ID NOs: 27-34 or SEQ ID NOs: 117-119. In some aspects, the seventh polypeptide linker and/or the eighth polypeptide linker comprise one or more repeats of the amino acid sequence of SEQ ID NO: 29 or SEQ ID NO: 117. In some aspects, the seventh polypeptide linker and the eighth polypeptide linker each comprise SEQ ID NO: 32. In some aspects, the seventh polypeptide linker and the eighth polypeptide linker each comprise SEQ ID NO: 118.

The present disclosure is also directed to a multivalent antibody binding molecule, comprising a first polypeptide, a second polypeptide, a third polypeptide, and a fourth polypeptide. In some aspects, the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 61. In some aspects, the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 80. In some aspects, the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 81. In some aspects, the first polypeptide comprises the amino acid sequence of SEQ ID NO: 87, the second polypeptide comprises the amino acid of SEQ ID NO: 88, and the third polypeptide and the fourth polypeptide each comprise the amino acid sequence of SEQ ID NO: 89. In some aspects, the first polypeptide comprises the amino acid sequence of SEQ ID NO: 90, the second polypeptide comprises the amino acid of SEQ ID NO: 91, and the third polypeptide and the fourth polypeptide each comprise the amino acid sequence of SEQ ID NO: 92. In some aspects, the first polypeptide comprises the amino acid sequence of SEQ ID NO: 93, the second polypeptide comprises the amino acid of SEQ ID NO: 94, and the third polypeptide and the fourth polypeptide each comprise the amino acid sequence of SEQ ID NO: 95. In some aspects, the first polypeptide comprises the amino acid sequence of SEQ ID NO: 96, the second polypeptide comprises the amino acid of SEQ ID NO: 97, and the third polypeptide and the fourth polypeptide each comprise the amino acid sequence of SEQ ID NO: 98. In some aspects, the first polypeptide comprises the amino acid sequence of SEQ ID NO: 99, the second polypeptide comprises the amino acid of SEQ ID NO: 100, and the third polypeptide and the fourth polypeptide each comprise the amino acid sequence of SEQ ID NO: 101. In some aspects, the first polypeptide comprises the amino acid sequence of SEQ ID NO: 99, the second polypeptide comprises the amino acid of SEQ ID NO: 100, and the third polypeptide and the fourth polypeptide each comprise the amino acid sequence of SEQ ID NO: 101.

In some aspects, the first polypeptide comprises the amino acid sequence of SEQ ID NO: 102, the second polypeptide comprises the amino acid of SEQ ID NO: 103, and the third polypeptide and the fourth polypeptide each comprise the amino acid sequence of SEQ ID NO: 104. In some aspects, the first polypeptide comprises the amino acid sequence of SEQ ID NO: 105, the second polypeptide comprises the amino acid of SEQ ID NO: 106, and the third polypeptide and the fourth polypeptide each comprise the amino acid sequence of SEQ ID NO: 107. In some aspects, the first polypeptide comprises the amino acid sequence of SEQ ID NO: 108, the second polypeptide comprises the amino acid of SEQ ID NO: 109, and the third polypeptide and the fourth polypeptide each comprise the amino acid sequence of SEQ ID NO: 110. In some aspects, the first polypeptide comprises the amino acid sequence of SEQ ID NO: 111, the second polypeptide comprises the amino acid of SEQ ID NO: 112, and the third polypeptide and the fourth polypeptide each comprise the amino acid sequence of SEQ ID NO: 113. In some aspects, the first polypeptide comprises the amino acid sequence of SEQ ID NO: 114, the second polypeptide comprises the amino acid of SEQ ID NO: 115, and the third polypeptide and the fourth polypeptide each comprise the amino acid sequence of SEQ ID NO: 116. In some aspects, the first polypeptide comprises the amino acid sequence of SEQ ID NO: 87, the second polypeptide comprises the amino acid of SEQ ID NO: 88, and the third polypeptide and the fourth polypeptide each comprise the amino acid sequence of SEQ ID NO: 89. In some aspects, the first polypeptide comprises the amino acid sequence of SEQ ID NO: 93, the second polypeptide comprises the amino acid of SEQ ID NO: 94, and the third polypeptide and the fourth polypeptide each comprise the amino acid sequence of SEQ ID NO: 95. In some aspects, the first polypeptide comprises the amino acid sequence of SEQ ID NO: 96, the second polypeptide comprises the amino acid of SEQ ID NO: 97, and the third polypeptide and the fourth polypeptide each comprise the amino acid sequence of SEQ ID NO: 98.

The present disclosure is also directed to a pharmaceutical composition comprising the multivalent antibody binding molecule described herein and a pharmaceutically acceptable carrier.

The present disclosure is also directed to a method of treating an ocular disorder comprising administering to a person in need thereof a therapeutically effective amount of the multivalent antibody binding molecule described herein or the pharmaceutical composition described herein. In some aspects, the ocular disorder is selected from diabetic retinopathy, retinopathy of prematurity, Coats' disease, Familial Exudative Vitreoretinopathy (FEVR), Norrie disease, macular degeneration, diabetic macular edema, and pediatric vitreoretinopathies. In some aspects, the ocular disorder is macular degeneration. In some aspects, the macular degeneration is neovascular age-related macular degeneration (nvAMD). In some aspects, the ocular disorder is diabetic macular edema. In some aspects, the multivalent antibody binding molecule described herein or the pharmaceutical composition described herein is administered by intravitreal injection.

The present disclosure is also directed to use of the multivalent antibody binding molecule described herein or the pharmaceutical composition described herein in the manufacture of a medicament for the treatment of an ocular disorder. In some aspects, the ocular disorder is selected from diabetic retinopathy, retinopathy of prematurity, Coats' disease, Familial Exudative Vitreoretinopathy (FEVR), Norrie disease, macular degeneration, diabetic macular edema, and pediatric vitreoretinopathies. In some aspects, the ocular disorder is macular degeneration. In some aspects, the macular degeneration is neovascular age-related macular degeneration (nvAMD). In some aspects, the ocular disorder is diabetic macular edema.

The present disclosure is also directed to a composition comprising the multivalent antibody binding molecule described herein for use in the treatment of an ocular disorder. In some aspects, the ocular disorder is selected from diabetic retinopathy, retinopathy of prematurity, Coats' disease, Familial Exudative Vitreoretinopathy (FEVR), Norrie disease, macular degeneration, diabetic macular edema, and pediatric vitreoretinopathies. In some aspects, the ocular disorder is macular degeneration. In some aspects, the ocular disorder is diabetic macular edema. In some aspects, the macular degeneration is neovascular age-related macular degeneration (nvAMD).

The present disclosure is also directed to a nucleic acid molecule encoding the multivalent antibody binding molecule described herein. In some aspects, the nucleic acid molecule encodes one or more of the first polypeptide, the second polypeptide, the third polypeptide, or the fourth polypeptide of the multivalent antibody binding molecule described herein.

The present disclosure is also directed to a vector comprising the nucleic acid molecule described herein. The present disclosure is also directed to a host cell comprising the nucleic acid molecule described herein. The present disclosure is also directed to a host cell comprising one or more vectors described herein.

The present disclosure is also directed to a method for producing the multivalent antibody binding molecule described herein, comprising providing the host cell described herein, culturing the host cell in culture medium under conditions to enable expression of the multivalent antibody binding molecule, and optionally isolating the multivalent antibody binding molecule.

### BRIEF DESCRIPTION OF THE DRAWINGS

FIG. 1 shows multivalent antibody binding molecule formats 1.0 and 2.2 described herein. Format 2.2 comprises VEGF receptor components attached to the light chains of the molecule.

FIG. 2 shows various multivalent antibody binding molecule formats described herein, including formats 3.1-3.6. The locations of the VEGF binding domain(s) are shown at the C-termini of the molecules as compared to format 1.0 (See FIG. 1)

FIGS. 3A and 3B show SDS-PAGE results after Protein A affinity column purification for the 3.1-3.6 (FIG. 3A) and 2.2 (FIG. 3B) constructs, as described in Example 1.

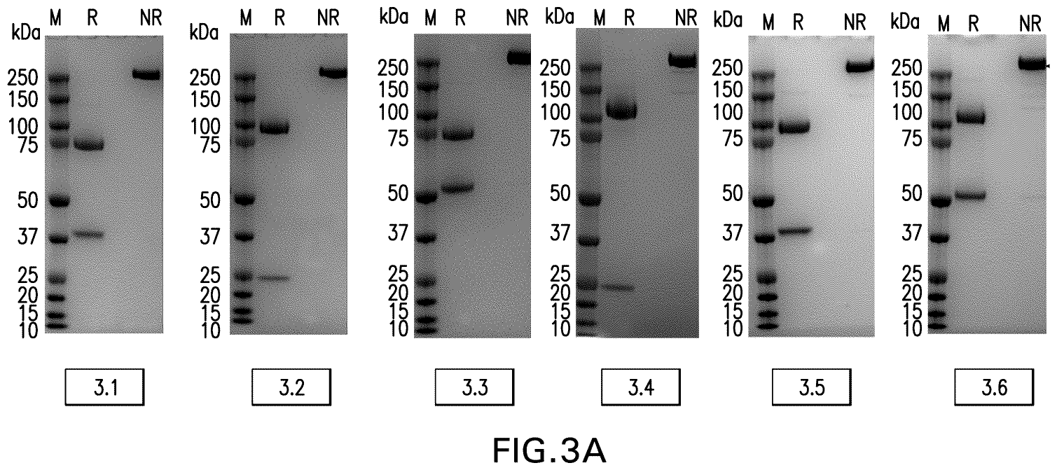

FIG. 4 shows a diagrammatic representation of the two lentiviral constructs used to create the CHO TopFLASH cell line as described in Example 1.

FIG. 5 shows an initial dose response curve for molecule 1.0, using a CHO cell TopFLASH assay as described in Example 1.

FIG. 6 shows a VEGF neutralization dose response curve for a reference standard as described in Example 1.

FIG. 7 shows Axin2 real-time PCR analysis of bEnd.3 cells after treatment with various agents as described in Example 3.

FIG. 8 shows the results from an in vitro functional assay demonstrating rescue of VEGF-induced hyper-permeability in mouse bEnd.3 cells as described in Example 4.

FIG. 9 shows a non-linear fit of the output of FIG. 8 comparing anti-VEGF alone, WNT signaling alone, and WNT signaling plus anti-VEGF treatment.

### DETAILED DESCRIPTION OF THE DISCLOSURE

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

As used herein, the term “activates” refers to a measurable increase in the intracellular level of a Wnt signaling pathway, e.g., the Wnt/β-catenin signaling pathway, compared with the level in the absence of a FZD Agonist of the disclosure.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the aspects “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

The term “antibody” as referred to herein includes whole antibodies. A “whole antibody” or full-length antibody refers to a glycoprotein comprising at least two heavy (H) chains and two light (L) chains inter-connected by disulfide bonds. Each heavy chain is comprised of a heavy chain variable region or domain (abbreviated herein as VH) and a heavy chain constant region. The heavy chain constant region is comprised of three domains, CH1, CH2 and CH3. Each light chain is comprised of a light chain variable region or domain (abbreviated herein as VL) and a light chain constant region. The light chain constant region is comprised of one domain, CL or CL1. The light chain constant region can be of kappa or lambda type. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector functions, e.g., binding Fc receptors and activation of antibody-dependent cellular cytotoxicity (ADCC) and complement dependent cytotoxicity (CDC). Three highly divergent stretches within each of the heavy chain variable domain, VH or VH domain, and light chain variable domain, VL or VL domain, referred to as complementarity determining regions (CDRs), are interposed between more conserved flanking stretches known as “framework regions”, or “FRs”. Thus, the term “FR” refers to amino acid sequences which are naturally found between, and adjacent to, CDRs in immunoglobulins. A VH domain typically has four FRs, referred to herein as VH framework region 1 (FR1), VH framework region 2 (FR2), VH framework region 3 (FR3), and VH framework region 4 (FR4). Similarly, a VL domain typically has four FRs, referred to herein as VL framework region 1 (FR1), VL framework region 2 (FR2), VL framework region 3 (FR3), and VL framework region 4 (FR4). In an antibody molecule, the three CDRs of a VL domain (CDR-L1, CDR-L2 and CDR-L3) and the three CDRs of a VH domain (CDR-H1, CDR-H2 and CDR-H3) are disposed relative to each other in three-dimensional space to form an antigen-binding site within the antibody variable region. The surface of the antigen-binding site is complementary to a three-dimensional surface of a bound antigen. The amino acid sequences of VL and VH domains may be numbered, and CDRs and FRs therein identified/defined, according to the Kabat numbering system (Kabat et al., 1991, Sequences of Proteins of Immunological Interest, 5th Ed. Public Health Service, National Institutes of Health, Bethesda, Md.) or the INTERNATIONAL IMMUNOGENETICS INFORMATION SYSTEM (IMGT numbering system; Lefranc et al., 2003, Development and Comparative Immunology 27:55-77), both incorporated herein by reference. One of ordinary skill in the art would possess the knowledge for numbering amino acid residues of a VL domain and of a VH domain, and identifying CDRs and FRs therein, according to a routinely employed numbering system such as the IMGT numbering system, the Kabat numbering system, and the like.

The term “antigen-binding portion” or “antigen-binding fragment” of an antibody (or simply “antibody fragment”), as used herein, refers to one or more fragments, portions or domains of an antibody that retain the ability to specifically bind to an antigen. It has been shown that fragments of a full-length antibody can perform the antigen-binding function of an antibody. Examples of binding fragments encompassed within the term “antigen-binding portion” of an antibody include (i) an Fab fragment, a monovalent fragment consisting of the VL, VH, CL1 and CH1 domains; (ii) an F(ab′)2 fragment, a bivalent fragment comprising two F(ab)′ fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment (Ward et al. (1989) Nature 241:544-546), which consists of a VH domain; and (vi) an isolated complementary determining region (CDR). Furthermore, although the two domains of the Fv fragment, VL and VH, are coded for by separate genes, they can be joined, using recombinant methods, by a synthetic linker that enables them to be made as a single contiguous chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies, are also encompassed (see e.g., Holliger et al. (1993) PNAS. USA 90:6444-6448). Among antibody fragments, Fab has one antigen-binding site with a structure of the variable regions of the light and heavy chain, the constant region of the light chain, and the first CH1 of the heavy chain. Fab′ differs from Fab in that it has a hinge region comprising one or more cysteine residues at the C-terminus of the CH1 domain. F(ab′)2 antibody is produced by disulfide bonds formation between Cysteine residues in the region of the hinge of Fab′. Fv is the smallest antibody fragment having only the variable region of the heavy chain and the variable region of the light chain Double chain Fv (two-chain Fv) is formed by a noncovalent bond between the heavy chain variable region and the light chain variable region, and single-chain Fv (scFv) is generally formed through a peptide linker covalently between the variable region of the heavy chain and the variable region of the light chain, or is connected directly at the C-terminus by forming a dimer-like structure like a double-chain Fv. This fragment can be obtained by protein hydrolysis enzyme (e.g., one can get Fab by restriction digestion of whole antibody using papain, one can get F(ab′)2 fragment by cutting with pepsin), also made by genetic manipulation technology. An antibody may be, for example, in the Fv form (e.g., scFv), or a complete antibody form. In addition, constant region of the heavy chain may be selected from any isotypes of gamma (γ), mu (μ), alpha (α), delta (δ), or epsilon (ε). For example, the constant region is gamma 1 (IgG1), gamma 3 (IgG3), or gamma 4 (IgG4). The light chain constant region can be of kappa (Cκ) or lambda (Cλ) type.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

“Diabodies,” or sometimes referred to herein as “Dia,” or “Db” are dimeric antibody fragments. In each polypeptide of the diabody, a heavy-chain variable domain (VH) is linked to a light-chain variable domain (VL), but unlike single-chain Fv fragments, the linker between the VL and VH is too short for intramolecular pairing and as such each antigen-binding site is formed by pairing of the VH and VL of one polypeptide with the VH and VL of the other polypeptide. Diabodies thus have two antigen-binding sites, and can be monospecific or bispecific. (see, e.g., Holliger, P., et al. (1993) Proc. Natl. Acad. Sci. USA 90:6444-6448; Poljak, R. J., et al. (1994) Structure 2:1121-1123; Kontermann and Dubel eds., Antibody Engineering (2001) Springer-Verlag. New York. 790 pp. (ISBN 3-540-41354-5) incorporated herein by reference.

“VHH domains”, also known as VHHs, or VHH antibodies, were originally described as the antigen binding immunoglobulin (variable) domain of “heavy chain antibodies” (i.e., of “antibodies devoid of light chains”; Hamers-Casterman C, Atarhouch T, Muyldermans S, Robinson G, Hamers C, Songa E B, Bendahman N, Hamers R.: “Naturally occurring antibodies devoid of light chains”; Nature 363, 446-448 (1993)). The term “VHH domain” was chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). VHH domains can specifically bind to an epitope without an additional antigen binding domain (as opposed to VH or VL domains in a conventional 4-chain antibody, in which case the epitope is recognized by a VL domain together with a VH domain). VHH domains are small, robust and efficient antigen recognition units formed by a single immunoglobulin domain.

“Single-domain antibody” (sdAb), or “nanobody”, is an antibody fragment consisting of a single monomeric variable antibody domain (VHH domain) and an antibody constant region.

As used herein an “effective amount” of an agent, e.g., the multivalent antibody binding molecules or a pharmaceutical composition comprising the molecules, refers to an amount effective, at dosages and for periods of time necessary, to achieve the desired result. In some aspects, a therapeutically effective amount is one that reduces the incidence and/or severity of, stabilizes one or more characteristics of, and/or delays onset of, one or more symptoms of a disease, disorder, and/or condition, e.g., diabetic macular edema (DME), neovascular age-related macular degeneration (nvAMD) or wet age-related macular degeneration.

As used herein, the term “epitope” includes any protein determinant capable of specific binding to an immunoglobulin or fragment thereof, or a T-cell receptor. Epitopic determinants usually consist of chemically active surface groupings of molecules such as amino acids or sugar side chains and usually have specific three-dimensional structural characteristics, as well as specific charge characteristics.

“Affinity” refers to the strength of the sum total of noncovalent interactions between a single binding site of a molecule (e.g., a multivalent antibody binding molecule) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including KinExA and Biacore.

In some aspects, the invention provides multivalent antibody binding molecules that specifically bind to a target (e.g., LRP5 or FZD4). An antibody is said to “specifically bind” to an antigen or target when the dissociation constant is ≤10 μM; e.g., ≤100 nM, ≤10 nM or ≤1 nM. In some aspects, an antibody that “specifically binds to the target,” or an antibody that “specifically binds to a polypeptide comprising the amino acid sequence of the target,” is an antibody or antigen-binding fragment thereof that binds to target with a K_{D} of about 1 nM or a higher binding affinity. For example, an antibody that specifically binds to a polypeptide comprising the target may bind to a FLAG®-tagged form of X but will not bind to other FLAG®-tagged proteins. In one aspect, the antibody or antigen-binding fragment thereof binds to human target with a K_{D} of about 1 nM-2 pM. In another aspect, the antibody or antigen-binding fragment thereof binds to human target with a K_{D} of about 100 pM or higher affinity.

The epitope for a given antibody (Ab)/antigen (Ag) pair can be defined and characterized at different levels of detail using a variety of experimental and computational epitope mapping methods. The experimental methods include mutagenesis, X-ray crystallography, Nuclear Magnetic Resonance (NMR) spectroscopy and Hydrogen deuterium exchange Mass Spectrometry (HX-MS), methods that are known in the art. As each method relies on a unique principle, the description of an epitope is intimately linked to the method by which it has been determined. Thus, depending on the epitope mapping method employed, the epitope for a given Ab/Ag pair will be described differently.

The epitope for a given antibody (Ab)/antigen (Ag) pair may be described by routine methods. For example, the overall location of an epitope may be determined by assessing the ability of an antibody to bind to different fragments or variants of the antigen. The specific amino acids within the antigen that make contact with an antibody (epitope) may also be determined using routine methods. For example, the Ab and Ag molecules may be combined and the Ab/Ag complex may be crystallized. The crystal structure of the complex may be determined and used to identify specific sites of interaction between the Ab and Ag.

The constant region of immunoglobulin molecules comprises a CH1, CH2 and CH3 domain, wherein the CH1 and CH2 domains are separated by a hinge region. Wild-type sequences for IgG1 CH1, CH2, and CH3 domains can be found in SEQ ID NO: 45, SEQ ID NO: 17, and SEQ ID NO: 20 respectively. The subunit of the constant domain comprising the CH2 and CH3 domains of the constant region of immunoglobulin molecules is also called the fragment crystallizable region, the “Fc region” or “Fc domain.” The Fc domain is composed of two identical protein fragments, derived from the second and third constant domains (CH2 and CH3) of the antibody's two heavy chains, and the Fc domains of IgGs bear a highly conserved N-glycosylation site. The CH2 domains described herein may or may not also comprise an IgG1 hinge region at the N-terminus of the CH2 domain, according to SEQ ID NO: 28. Glycosylation of the Fc fragment is essential for Fc receptor-mediated activity. In an aspect of the disclosure, the Fc domain of the multivalent antibody binding molecule is engineered such that it does not target the cell that binds the multivalent antibody binding molecule for ADCC or CDC-dependent death. In an aspect of the disclosure, the Fc domain of the multivalent antibody binding molecule is a peptide dimer in a knob-in-hole configuration. The peptide dimer may be a heterodimer.

The terms “individual,” “subject,” “host,” and “patient,” are used interchangeably herein and refer to any mammalian subject for whom diagnosis, treatment, or therapy is desired, particularly humans.

“LRP”, “LRP proteins” and “LRP receptors” is used herein to refer to members of the low-density lipoprotein receptor-related protein family. These receptors are single-pass transmembrane proteins that bind and internalize ligands in the process of receptor-mediated endocytosis. LRP proteins LRP5 (e.g., LRP5: NP_002326.2) and LRP6 (e.g., LRP6: NP_002327.2) are included in a Wnt receptor complex required for activation on the Wnt/β-catenin signaling pathway. See also, for human/mouse LRP5 and LRP6: https://www.uniprot.org/uniprot/O75197, https://www.uniprot.org/uniprot/Q91VN0, https://www.uniprot.org/uniprot/O75581, https://www.uniprot.org/uniprot/O88572.

The term “polypeptide fragment” as used herein refers to a polypeptide that has an amino terminal and/or carboxy-terminal deletion, but where the remaining amino acid sequence is identical to the corresponding positions in the naturally occurring sequence deduced, for example, from a full-length cDNA sequence.

As used herein the term “paratope” includes the antigen binding site in the variable region of an antibody that binds to an epitope.

“Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv and other antibody fragments, see James D. Marks, Antibody Engineering, Chapter 2, Oxford University Press (1995) (Carl K. Borrebaeck, Ed.).

The terms “treatment”, “treating” and the like are used herein to generally mean to administer a therapeutic moiety, such as a composition comprising any of the multivalent antibody binding molecules of the disclosure, to a subject or patient having one or more disease symptoms, suspected of having a disease, or diagnosed with a disease, for which the therapeutic moiety has therapeutic activity to obtain a desired pharmacologic and/or physiologic effect. The effect may be a partial or complete cure for a disease and/or adverse effect attributable to the disease. “Treatment” as used herein covers any treatment of a disease in a mammal and includes: (a) alleviating one or more symptoms of a disease; (b) inhibiting the disease, i.e., slowing or arresting its development; or (c) relieving the disease, i.e., causing regression of the disease. The therapeutic agent may be administered during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease. Treatment with a multivalent antibody binding molecule of the invention may be combined with other interventions to treat other symptoms or diseases or to provide additional therapeutic efficacy against the same disease or disorder for which the multivalent antibody binding molecule is meant to treat.

As used herein, a subject “in need of treatment” means a subject diagnosed with or suspected of having (e.g., exhibiting a disease symptom) a disease (e.g., diabetic macular edema or macular degeneration), in either case, that will be the subject of the treatment.

As used herein, the term “prevention” denotes any action to inhibit, delay the onset of, or reduce the likelihood of occurrence of the disease of interest by administering the multivalent antibody binding molecule or composition.

The term “heavy chain” or “HC” as used herein means the full-length heavy chain or fragments thereof comprising a variable region domain VH and three constant region domains CH1, CH2 and CH3, having an amino acid sequence with a sufficient variable region in order to provide antigen specificity.

The term “light chain” or “LC”, means the full-length light chain or fragments thereof comprising a variable region domain VL and a constant region domain CL, having an amino acid sequence with a sufficient variable region in order to provide antigen specificity.

The “humanized” form of non-human (e.g., murine) antibody is a chimeric antibody comprising one or more amino acid sequence (e.g., one or more CDR sequences, such as six CDR sequences) from a non-human antibody (donor or source antibody) and otherwise minimal sequence derived from non-human immunoglobulins. In some aspects, the humanized antibody is a human immunoglobulin (receptor antibody) whose hypervariable regions are replaced by residues from hypervariable regions of non-human primates, mouse, rat, rabbit or non-human primate (receptor antibody), possessing the desired specificity, affinity and ability of residues from the hypervariable region of the recipient. For humanization one or more residues in the framework domain (FR) can be replaced by the corresponding residue of the non-human donor antibody. This can help to maintain a proper three-dimensional configuration of the grafted CDR(s), thereby improving affinity and antibody stability. Humanized antibodies, e.g., can alternatively or additionally include a new residue that does not appear in the original recipient antibody or donor antibody to further refine additional performance of antibody.

“Antibody variable domain” as used herein refers to a domain comprising complementarity determining regions (CDRs; e.g., CDR1, CDR2, and CDR3) and the framework regions (FRs). VH refers to the variable domain of the heavy chain VL refers to the variable domain of the light chain. “Framework region” (FR) is a variable domain segment outside of the CDRs. Each variable domain typically has 4 FRs identified as FR1, FR2, FR3 and FR4.

“Complementarity determining regions” (CDRs; i.e., CDR1, CDR2, and CDR3) refer to the amino acid residues of the variable domain of the antibody, which are necessary for antigen binding. Each variable domain typically comprises three CDR regions identified as CDR1, CDR2 and CDR3.

As used herein, “or” has the inclusive sense (i.e., equivalent to and/or) unless the context clearly indicates otherwise.

As used herein, “antibody binding molecule” encompasses any polypeptide comprising sequences from multiple sources. Antibody binding molecules may be produced, e.g., from a genetic fusion (e.g., a polynucleotide encoding the sequence of the multivalent antibody binding molecule) or by chemically joining polypeptides that were produced or synthesized separately.

As used herein “EU index”, “EU numbering” or “EU index as in Kabat” or “EU numbering scheme” refers to the numbering of the EU antibody (Edelman et al., 1969, Proc Natl Acad Sci USA 63:78-85, hereby entirely incorporated by reference.)

“Angiogenic disease,” also referred to as “angiogenesis-related disease,” means the occurrence of angiogenesis or a disease associated with progression of angiogenesis.

A “linker,” as used herein, is a construct that links two polypeptides. In certain aspects, the linker is a peptide linker and links two polypeptides. A linker can be used to link two immunoglobulin variable regions (e.g., VH and VL) in the context of a multispecific binding construct.

A “hinge region”, as used herein, refers to an amino acid sequence found in an antibody constant domain (CH1, CH2, CH3), that separates the CH1 and CH2 regions and provides structural flexibility. The hinge region forms a flexible linker between the Fab arms and the Fc region of an antibody. Length and flexibility of the hinge region varies extensively among the IgG subclasses. The native hinge sequence of IgG1 encompasses 13 amino acids (SEQ ID NO: 28). The CH2 domains as described herein may also comprise a hinge region at the N-terminus according to SEQ ID NO: 28.

An “IgG1 upper hinge region”, as used herein, refers to a subset of amino acid residues located near the N-terminus of the antibody hinge region. The IgG1 upper hinge region is found in SEQ ID NO: 27.

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Nucleic acid molecule” is meant to encompass DNA (gDNA and cDNA) and RNA molecules inclusively, and nucleotide, the basic structural unit of nucleic acid, includes the nucleotide in nature, as well as the analogue with modified sugar or base moieties. The sequence of the nucleic acid encoding the heavy and light chain variable regions can be modified. The modifications include addition, deletion, or non-conservative or conservative substitution of nucleotides. In specific aspects, a multivalent antibody binding molecule of the disclosure is encoded by a single nucleic acid molecule. In alternative aspects, each chain of a multivalent antibody binding molecule of the disclosure is encoded by a separate nucleic acid molecule.

As used in this disclosure, the term “vector” refers to a nucleic acid delivery vehicle or plasmid that can be engineered to contain a nucleic acid molecule, e.g., a nucleic acid sequence encoding the multivalent antibody binding molecules described herein. The vector that can express protein when inserted with a polynucleotide is called an expression vector. Vectors can be inserted into the host cell by transformation, transduction, or transfection, so that the carried genetic substances can be expressed in the host cell. Vectors are well known to the technical personnel in the field, including but not limited to: plasmid, phagemid, cosmid, artificial chromosome such as yeast artificial chromosome (YAC), bacterial artificial chromosome (BAC), or P1 derived artificial chromosome (PAC), phage such as λ phage or M13 phage and animal viruses etc. Animal viruses may include but not limited to, reverse transcriptase virus (including lentivirus), adenovirus, adeno-associated virus, herpes virus (e. g. herpes simplex virus), varicella-zoster virus, baculovirus, papillomavirus, and papovavirus (such as SV40). A vector can contain multiple components that control expression of the multivalent antibody binding molecules described herein, including but not limited to, promoters, e.g., viral or eukaryotic promoters, e.g., a CMV promoter, signal peptides, e.g., TRYP2 signal peptide, transcription initiation factor, enhancer, selection element, and reporter gene. In addition, the vector may also contain replication initiation site(s). In an aspect of this disclosure, the vector comprises a nucleic acid encoding a heavy chain of the multivalent antibody binding molecule of the disclosure. In an aspect, the vector comprises a nucleic acid encoding a light chain of the multivalent antibody binding molecule of the disclosure. In an aspect, the vector comprises nucleic acids encoding two heavy chain sequences and one light chain sequence. An aspect of the disclosure is a set of one or more vectors which collectively comprise the set of one or more polynucleotides described previously, such that all chains of the multivalent antibody binding molecule of the disclosure are encoded in the set of vectors.

As used herein, the terms “operatively linked” or “linked” mean the functional linkage between a nucleic acid expression control sequence (for example, promoter, signal sequence, or array of transcriptional regulator binding sites) and different nucleic acid sequences, Thereby, the control sequence controls transcription and/or translation of the other nucleic acid sequence.

As used in this disclosure, the term “host cell” refers to cells that can import expression cassettes and vectors, including but not limited to, prokaryotic cells such as Escherichia coli and Bacillus subtilis, fungal cells such as yeast and Aspergillus, insect cells such as S2 drosophila cells and Sf9, or animal cells, including human cells, e.g., fibroblast cells, CHO cells, COS cells, NSO cells, HeLa cells, BHK cells, or HEK293 cells. An aspect of this disclosure is a host cell expressing a vector of the disclosure. An aspect of this disclosure is a process for the production of a multivalent antibody binding molecule of the disclosure using a vector.

### Molecules

The present disclosure is related to an antibody molecule that binds lipoprotein receptor-related protein 5 (LRP5), Frizzled 4 (FZD4), and vascular endothelial growth factor (VEGF), comprising: (a) a bivalent LRP5 binding domain, (b) an Fc domain, (c) a bivalent FZD4 binding domain, and (d) one or more VEGF binding domains; wherein the LRP5 binding domain is attached to the N-terminus of the Fc domain and the FZD4 binding domain is attached to the C-terminus the Fc domain, wherein the LRP5 binding domain comprises a diabody that binds LRP5, and wherein the FZD4 binding domain comprises two scFv molecules or two Fab molecules that bind FZD4.

As described herein, the FZD-binding Fabs or scFvs are formed by the pairing of each VH with a VL such that the VH that binds FZD4 pairs with the VL that binds FZD4 to form a FZD4 binding site.

As described herein, the LRP5-binding domains are formed by the pairing of a VH that binds LRP5 and a VL that binds LRP5 to form one or more LRP5 binding sites. In the case of a diabody format, the LRP5 binding domain comprises two LRP5 binding sites. In some aspects, the LRP5 binding sites are monospecific. In some aspects, the LRP5 binding sites are biparatopic.

The present disclosure is related to a multivalent antibody binding molecule comprising an LRP5 binding domain that specifically binds low-density lipoprotein receptor-related protein 5 (LRP5), an Fc domain, an FZD4 binding domain that specifically binds Frizzled 4 (FZD4), and one or more VEGF binding domains that each specifically bind vascular endothelial growth factor (VEGF); wherein the multivalent antibody molecule comprises four polypeptides, wherein the first polypeptide comprises: a first VH comprising a CDR-H1 having the amino acid sequence of SEQ ID NO: 1, a CDR-H2 having the amino acid sequence of SEQ ID NO: 2, and a CDR-H3 having the amino acid sequence of SEQ ID NO: 3; a VL comprising a CDR-L1 having the amino acid sequence of SEQ ID NO: 10, a CDR-L2 having the amino acid sequence of SEQ ID NO: 11, and a CDR-L3 having the amino acid sequence of SEQ ID NO: 12; and a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37; the second polypeptide comprises: a first VH comprising a CDR-H1 having the amino acid sequence of SEQ ID NO: 7, a CDR-H2 having the amino acid sequence of SEQ ID NO: 8, and a CDR-H3 having the amino acid sequence of SEQ ID NO: 9; a VL comprising a CDR-L1 having the amino acid sequence of SEQ ID NO: 4, a CDR-L2 having the amino acid sequence of SEQ ID NO: 5, and a CDR-L3 having the amino acid sequence of SEQ ID NO: 6; and a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37; and the third polypeptide and the fourth polypeptide each comprise a VL comprising a CDR-L1 having the amino acid sequence of SEQ ID NO: 38, a CDR-L2 having the amino acid sequence of SEQ ID NO: 39, and a CDR-L3 having the amino acid sequence of SEQ ID NO: 40, and wherein each of the VEGF binding domains comprise a VEGF receptor component.

The present disclosure is also related to a multivalent antibody binding molecule, comprising an LRP5 binding domain that specifically binds low-density lipoprotein receptor-related protein 5 (LRP5), an Fc domain, an FZD4 binding domain that specifically binds Frizzled 4 (FZD4), and one or more VEGF binding domains that each specifically bind vascular endothelial growth factor (VEGF); wherein the LRP5 binding domain is attached to the N-terminus of the Fc domain and the FZD4 binding domain is attached to the C-terminus the Fc domain, the LRP5 binding domain comprises a diabody that binds LRP5, the FZD4 binding domain comprises two scFv molecules or two Fab molecules that bind FZD4, and each of the VEGF binding domains comprise one or more single heavy chain variable domain antibodies (VHH) or antigen binding fragments thereof, wherein each VHH comprises a heavy chain complementarity determining region 1 (HC-CDR1) comprising the amino acid sequence set forth in SEQ ID NO: 50 or 54; an HC-CDR2 comprising the amino acid sequence set forth in SEQ ID NO: 51 or 55; and an HC-CDR3 comprising the amino acid sequence set forth in SEQ ID NO: 52 or 56.

In some aspects, the FZD4 binding domain comprises two Fab molecules that bind FZD4. In some aspects, the one or more VEGF binding domains each comprise an antibody or antigen binding fragment thereof, a VEGF receptor component, or a combination thereof. In some aspects, the multivalent antibody binding molecule comprises a first VEGF binding domain that is attached to the C-terminus of one or both of the scFv molecules or the Fab molecules. In some aspects, the FZD4 binding domain comprises two Fab molecules that bind FZD4, and wherein the multivalent antibody binding molecule comprises a first VEGF binding domain and second VEGF binding domain, wherein each VEGF binding domain is attached to the C-terminus of each of the Fab molecules.

In some aspects, the first VEGF binding domain is attached to the C-terminus of the first polypeptide, and the second VEGF binding domain is attached to the C-terminus of the second polypeptide. In some aspects, the first VEGF binding domain is attached to the C-terminus of the third polypeptide, and the second VEGF binding domain is attached to the C-terminus of the fourth polypeptide.

In some aspects, the FZD4 binding domain comprises two Fab molecules that bind FZD4, and wherein the multivalent antibody binding molecule comprises a first VEGF binding domain and second VEGF binding domain, wherein each VEGF binding domain is attached to the N-terminus of the diabody that binds LRP5. In some aspects, the first VEGF binding domain is attached to the N-terminus of the first polypeptide, and the second VEGF binding domain is attached to the N-terminus of the second polypeptide.

Described herein are multivalent antibody binding molecules comprising an Fc domain, with or without effector function, a bivalent FZD4 binding domain and a bivalent LRP5-binding domain, wherein the binding domains are attached to opposite ends of the Fc domain. In an aspect, the FZD binding domain is attached to the carboxy terminus of the Fc region and the LRP co-receptor binding domain is attached to the amino terminus of the Fc domain. Alternatively, the FZD binding domain is attached to the amino terminus of the Fc region and the co-receptor binding domain is attached to the carboxy terminus of the Fc domain. The binding domains may be attached directly to the Fc domain or attached to the Fc domain via a linker.

In an aspect of the disclosure, the FZD binding domain is bivalent and comprises a diabody or comprises a scFv, a V_{H}H fragment, or a Fab fragment or combinations thereof that bind FZD4, and the co-receptor binding domain is bivalent and comprises a diabody or a V_{H}H fragment, an Fab, or a scFv or combinations thereof that bind the LRP5 receptor. In an aspect of the disclosure the FZD binding domain is attached to the carboxy-terminus of the Fc domain and comprises two scfv, two V_{H}H fragments, two Fab fragments or a diabody that bind FZD, and the co-receptor binding domain attached to the amino terminus of the Fc domain comprises a diabody, two V_{H}H fragments or two scFvs that binds to the LRP5 receptor.

When attached to the carboxy terminus of the Fc domain, the FZD-binding Fabs are linked to the CH3 of the Fc domain via the Fab variable heavy region or variable light region. In other aspects, the FZD binding domain is attached to the amino terminus of the Fc domain and is comprised of two Fabs and the LRP5 receptor binding domain is attached to the carboxy terminus of the Fc domain and is comprised of a diabody or two scFvs that bind the co-receptor. The present disclosure is related to an antibody molecule that binds lipoprotein receptor-related protein 5 (LRP5), Frizzled 4 (FZD4), and vascular endothelial growth factor (VEGF), comprising: (a) a bivalent LRP5 binding domain, (b) an Fc domain, (c) a bivalent FZD4 binding domain, and (d) one or more VEGF binding domains; wherein the LRP5 binding domain is attached to the N-terminus of the Fc domain and the FZD4 binding domain is attached to the C-terminus the Fc domain, wherein the LRP5 binding domain comprises a diabody that binds LRP5, and wherein the FZD4 binding domain comprises two scFv molecules or two Fab molecules that bind FZD4. In some aspects, the FZD4 binding domain comprises two Fab molecules that bind FZD4.

In some aspects, the multivalent antibody binding molecule comprises: (a) a first polypeptide comprising, from N-terminus to C-terminus, a first heavy chain variable domain (VH), a light chain variable domain (VL), a constant heavy chain domain 2 (CH2) and a constant heavy chain domain 3 (CH3) of the Fc domain, a second VH, and a constant heavy chain domain 1 (CH1), (b) a second polypeptide comprising, from N-terminus to C-terminus, a first VH, a VL, a CH2 and a CH3 of the Fc domain, a second VH, and a CH1, (c) a third polypeptide comprising, from N-terminus to C-terminus, a light chain variable domain (VL) and a constant light chain domain 1 (CL1); and (d) a fourth polypeptide comprising, from N-terminus to C-terminus, a VL and a CL1.

### VEGF Antagonists

The molecules of the present disclosure comprise a VEGF binding domain. In some aspects the VEGF binding domain described herein is a VEGF antagonist. As used herein, the expression “VEGF antagonist” means any molecule that blocks, reduces or interferes with the normal biological activity of VEGF.

VEGF antagonists include molecules which interfere with the interaction between VEGF and a natural VEGF receptor, e.g., molecules which bind to VEGF or a VEGF receptor and prevent or otherwise hinder the interaction between VEGF and a VEGF receptor. Specific exemplary VEGF antagonists include anti-VEGF antibodies, anti-VEGF receptor antibodies, and VEGF receptor-based chimeric molecules (also referred to herein as “VEGF-Traps”). VEGF receptor-based chimeric molecules include chimeric polypeptides which comprise two or more immunoglobulin (Ig)-like domains of a VEGF receptor such as VEGFR1 (also referred to as Flt1) and/or VEGFR2 (also referred to as Flk1 or KDR).

VEGF antagonists also include antibodies, antigen binding fragments thereof, or single heavy chain antibodies. The term “immunoglobulin single variable domain” as used herein means an immunoglobulin variable domain which is capable of specifically binding to an epitope of the antigen without pairing with an additional variable immunoglobulin domain. One example of immunoglobulin single variable domains in the meaning of the present disclosure are “domain antibodies”, such as the immunoglobulin single variable domains VH and VL (VH domains and VL domains). Another example of immunoglobulin single variable domains is a “VHH domain” (or simply “VHH”) from camelids, as defined hereinafter.

In some aspects, the first VEGF binding domain and/or the second VEGF binding domain comprises a VEGF receptor 1 (VEGFR1) extracellular Ig domain 2 and a VEGF receptor 2 (VEGFR2) extracellular Ig domain number 3.

In some aspects, the first VEGF binding domain and/or the second VEGF binding domain comprise one or more antibodies or antigen binding fragments thereof; or one or more single heavy chain antibodies, single heavy chain variable domain antibodies (VHH).

In some aspects, the first VEGF binding domain and/or the second VEGF binding domain comprise a VEGF receptor 1 (VEGFR1) extracellular Ig domain 2, and VEGF receptor 2 (VEGFR2) extracellular Ig domain number 3, and wherein the first VEGF binding domain is fused to the C-terminus of the first polypeptide and/or the second VEGF binding domain is fused to the C-terminus of the second polypeptide. In some aspects, the first VEGF binding domain and/or the second VEGF binding domain comprise a VEGF receptor 1 (VEGFR1) extracellular Ig domain 2, and VEGF receptor 2 (VEGFR2) extracellular Ig domain number 3, and wherein the first VEGF binding domain is fused to the C-terminus of the third polypeptide and/or the second VEGF binding domain is fused to the C-terminus of the fourth polypeptide. In some aspects, the first VEGF binding domain and the second VEGF binding domain comprise a VEGF receptor 1 (VEGFR1) extracellular Ig domain 2, and VEGF receptor 2 (VEGFR2) extracellular Ig domain number 3, and wherein the first VEGF binding domain is fused to the C-terminus of the third polypeptide and the second VEGF binding domain is fused to the C-terminus of the fourth polypeptide. In some aspects, the VEGF binding domain comprises an amino acid sequence at least 90% identical to any one of SEQ ID NOs: 47-49. In some aspects, the VEGF binding domain comprises the amino acid sequence of SEQ ID NO: 47-49. In some aspects, the VEGF binding domain comprises the amino acid sequence of SEQ ID NO: 49. In some aspects, the VEGF binding domain comprises SEQ ID NO: 49 and further comprises a linker at either the N-terminus or the C-terminus of the VEGF binding domain. In some aspects, the VEGF binding domain comprises SEQ ID NO: 49 and further comprises a 2×G4S linker at either the N-terminus or the C-terminus of the VEGF binding domain. In some aspects, the VEGF binding domain comprises SEQ ID NO: 49 and further comprises two repeats of a G4S linker (SEQ ID NO: 29) at either the N-terminus or the C-terminus of the VEGF binding domain. In some aspects, the VEGF binding domain comprises SEQ ID NO: 49 and further comprises three repeats of a G4S linker (SEQ ID NO: 29) at either the N-terminus or the C-terminus of the VEGF binding domain. In some aspects, the VEGF binding domain comprises SEQ ID NO: 49 and further comprises four repeats of a G4S linker (SEQ ID NO: 29) at either the N-terminus or the C-terminus of the VEGF binding domain. In some aspects, the VEGF binding domain comprises SEQ ID NO: 49 and further comprises two repeats of a G4S linker (SEQ ID NO: 29) at the N-terminus of the VEGF binding domain. In some aspects, the VEGF binding domain comprises SEQ ID NO: 49 and further comprises three repeats of a G4S linker (SEQ ID NO: 29) at the N-terminus of the VEGF binding domain.

In some aspects, the first VEGF binding domain and/or the second VEGF binding domain comprise one or more antibodies or antigen binding fragments thereof; or one or more single heavy chain antibodies (VHH) or antigen binding fragments thereof; and wherein the first VEGF binding domain is fused to the C-terminus of the first polypeptide and/or the second VEGF binding domain is fused to the C-terminus of the second polypeptide. In some aspects, the first VEGF binding domain and/or the second VEGF binding domain comprise one or more antibodies or antigen binding fragments thereof; or one or more single heavy chain variable domain antibodies (VHH) or antigen binding fragments thereof; and wherein the first VEGF binding domain is fused to the C-terminus of the third polypeptide and/or the second VEGF binding domain is fused to the C-terminus of the fourth polypeptide. In some aspects, the first VEGF binding domain and the second VEGF binding domain each comprise one or two single heavy chain variable domain antibodies (VHH); and wherein the first VEGF binding domain is fused to the C-terminus of the first polypeptide and the second VEGF binding domain is fused to the C-terminus of the second polypeptide. In some aspects, first VEGF binding domain and/or the second VEGF binding domain comprise: (a) a heavy chain complementarity determining region 1 (HC-CDR1), wherein the HC-CDR1 comprises the amino acid sequence set forth in SEQ ID NO: 50; an HC-CDR2, wherein the HC-CDR2 comprises the amino acid sequence set forth in SEQ ID NO: 51; an HC-CDR3, wherein the HC-CDR3 comprises the amino acid sequence set forth in SEQ ID NO: 52; or a HC-CDR1, wherein the HC-CDR1 comprises the amino acid sequence set forth in SEQ ID NO: 54; an HC-CDR2, wherein the HC-CDR2 comprises the amino acid sequence set forth in SEQ ID NO: 55; an HC-CDR3, wherein the HC-CDR3 comprises the amino acid sequence set forth in SEQ ID NO: 56. In some aspects, the first VEGF binding domain and/or the second VEGF binding domain each comprise an amino acid sequence that is at least 90% identical to SEQ ID NO: 53 or SEQ ID NO: 57. In some aspects, the first VEGF binding domain and/or the second VEGF binding domain each comprises an amino acid sequence selected from SEQ ID NO: 53 or SEQ ID NO: 57.

The present disclosure is also directed to a multivalent antibody binding molecule, comprising an LRP5 binding domain that specifically binds low-density lipoprotein receptor-related protein 5 (LRP5), an Fc domain, an FZD4 binding domain that specifically binds Frizzled 4 (FZD4), and one or more VEGF binding domains that each specifically bind vascular endothelial growth factor (VEGF); wherein: the LRP5 binding domain is attached to the N-terminus of the Fc domain and the FZD4 binding domain is attached to the C-terminus the Fc domain, the LRP5 binding domain comprises a diabody that binds LRP5, the FZD4 binding domain comprises two scFv molecules or two Fab molecules that bind FZD4, and each of the VEGF binding domains comprise one or more single heavy chain variable domain antibodies (VHH) or antigen binding fragments thereof, wherein each VHH or antigen binding fragment comprises (i) a heavy chain complementarity determining region 1 (HC-CDR1) comprising the amino acid sequence set forth in SEQ ID NO: 50; an HC-CDR2 comprising the amino acid sequence set forth in SEQ ID NO: 51; and an HC-CDR3 comprising the amino acid sequence set forth in SEQ ID NO: 52; or (ii) an HC-CDR1 comprising the amino acid set forth in SEQ ID NO: 54, an HC-CDR2 comprising the amino acid sequence set forth in SEQ ID NO:55 and the HC-CDR3 comprising the amino acid sequence set forth in SEQ ID NO: 56. In some aspects, each of the VEGF binding domains comprise one or more VHH. In some aspects, the multivalent antibody binding molecule comprises a first VEGF binding domain that is attached to the C-terminus of one or both of the scFv molecules or the Fab molecules.

In some aspects, the FZD4 binding domain comprises two Fab molecules that bind FZD4, and wherein the multivalent antibody binding molecule comprises a first VEGF binding domain and second VEGF binding domain, wherein each VEGF binding domain is attached to the C-terminus of each of the Fab molecules. In some aspects, the FZD4 binding domain comprises two Fab molecules that bind FZD4, and wherein the multivalent antibody binding molecule comprises a first VEGF binding domain and second VEGF binding domain, wherein each VEGF binding domain is attached to the N-terminus of each of the LRP5 binding domains. In some aspects, the first VEGF binding domain and the second VEGF binding domain are each attached to the C-terminus of each of the Fab molecules, and wherein the multivalent antibody binding molecule further comprises a third VEGF binding domain and a fourth VEGF binding domain, wherein the third VEGF binding domain and the fourth VEGF binding domain are each attached to the N-terminus of each of the LRP5 binding domains.

In some aspects, the multivalent antibody binding molecule comprises: (a) a first polypeptide comprising, from N-terminus to C-terminus, a first heavy chain variable domain (VH), a light chain variable domain (VL), a constant heavy chain domain 2 (CH2) and a constant heavy chain domain 3 (CH3) of the Fc domain, a second VH, and a constant heavy chain domain 1 (CH1); a second polypeptide comprising, from N-terminus to C-terminus, a first VH, a VL, a CH2 and a CH3 of the Fc domain, a second VH, and a CH1; a third polypeptide comprising, from N-terminus to C-terminus, a light chain variable domain (VL) and a constant light chain domain 1 (CL1); and a fourth polypeptide comprising, from N-terminus to C-terminus, a VL and a CL1. In some aspects, the first VEGF binding domain is fused to the C-terminus of the first polypeptide or the second polypeptide. In some aspects, the first VEGF binding domain is fused to the C-terminus of the first polypeptide and the second VEGF binding domain is fused to the C-terminus of the second polypeptide. In some aspects, the first VEGF binding domain is fused to the C-terminus of the third polypeptide and/or the second VEGF binding domain is fused to the C-terminus of the fourth polypeptide. In some aspects, the first VEGF binding domain is fused to the N-terminus of the first polypeptide or the second polypeptide. In some aspects, the first VEGF binding domain is fused to the N-terminus of the first polypeptide and the second VEGF binding domain is fused to the N-terminus of the second polypeptide.

In some aspects, the first VEGF binding domain and the second VEGF binding domain are each attached to the C-terminus of the first polypeptide and the second polypeptide, respectively, and wherein the multivalent antibody binding molecule further comprises a third VEGF binding domain, wherein the third VEGF binding domain is attached to the N-terminus of the first polypeptide or the second polypeptide. In some aspects, the first VEGF binding domain and the second VEGF binding domain are each attached to the C-terminus of the first polypeptide and the second polypeptide, respectively, and wherein the multivalent antibody binding molecule further comprises a third VEGF binding domain and a fourth VEGF binding domain, wherein the third VEGF binding domain is attached to the N-terminus of the first polypeptide and the fourth VEGF binding domain is attached to the N-terminus of the second polypeptide. In some aspects, each VEGF binding domain each comprises one VHH or antigen binding fragment thereof. In some aspects, each VEGF binding domain each comprises two VHH molecules or antigen binding fragments thereof. In some aspects, the first VEGF binding domain and/or the second VEGF binding domain each comprise one VHH or antigen binding fragment thereof. In some aspects, the first VEGF binding domain and the second VEGF binding domain each comprise two VHHs or antigen binding fragments thereof. In some aspects, the third VEGF binding domain and the fourth VEGF binding domain each comprise one VHH. In some aspects, the third VEGF binding domain and the fourth VEGF binding domain each comprise two VHHs.

In some aspects, each VHH comprises an amino acid sequence that is at least 90% identical to SEQ ID NO: 53 or SEQ ID NO: 57. In some aspects, each VHH comprises an amino acid sequence that is at least 95% identical to SEQ ID NO: 53 or SEQ ID NO: 57. In some aspects, each VHH comprises an amino acid sequence that is at least 96% identical to SEQ ID NO: 53 or SEQ ID NO: 57. In some aspects, each VHH comprises an amino acid sequence that is at least 97% identical to SEQ ID NO: 53 or SEQ ID NO: 57. In some aspects, each VHH comprises an amino acid sequence that is at least 98% identical to SEQ ID NO: 53 or SEQ ID NO: 57. In some aspects, each VHH comprises an amino acid sequence that is at least 99% identical to SEQ ID NO: 53 or SEQ ID NO: 57. In some aspects, each VHH comprises an amino acid sequence according to SEQ ID NO: 53 or SEQ ID NO: 57. In some aspects, each VHH comprises a variant amino acid sequence relative to SEQ ID NO:53 or SEQ ID NO:57, wherein the variant amino acid sequence differs from SEQ ID NO: 53 or SEQ ID NO: 57 by 5 or less conservative amino acid substitutions, 4 or less conservative amino acid substitutions, 3 or less conservative amino acid substitutions, 2 or less conservative amino acid substitutions or 1 conservative amino acid substitution.

In some aspects, the VHHs are separated by a VHH linker. In some aspects, the VHH linker comprises one or more of the amino acid sequence of SEQ ID NO: 29. In some aspects, the VHH linker comprises two repeats of SEQ ID NO: 29. In some aspects, the first VEGF binding domain, the second VEGF binding domain, the third VEGF binding domain, and/or the fourth VEGF binding domain comprise two VHHs and a VHH linker, and comprise a sequence according to SEQ ID NO: 82 or SEQ ID NO: 83.

In some aspects, the first VEGF binding domain and the second VEGF binding domain each comprise two VHHs and comprise an amino acid sequence that is at least 90% identical to SEQ ID NO: 82. In some aspects, the first VEGF binding domain and the second VEGF binding domain each comprise two VHHs and comprise an amino acid sequence that is at least 95% identical to SEQ ID NO: 82. In some aspects, the first VEGF binding domain and the second VEGF binding domain each comprise two VHHs and comprise an amino acid sequence that is at least 97% identical to SEQ ID NO: 82. In some aspects, the first VEGF binding domain and the second VEGF binding domain each comprise two VHHs and comprise an amino acid sequence that is at least 98% identical to SEQ ID NO: 82. In some aspects, the first VEGF binding domain and the second VEGF binding domain each comprise two VHHs and comprise an amino acid sequence according to SEQ ID NO: 82.

In some aspects, the first VEGF binding domain and the second VEGF binding domain each comprise two VHHs and comprise an amino acid sequence that is at least 90% identical to SEQ ID NO: 83. In some aspects, the first VEGF binding domain and the second VEGF binding domain each comprise two VHHs and comprise an amino acid sequence that is at least 95% identical to SEQ ID NO: 83. In some aspects, the first VEGF binding domain and the second VEGF binding domain each comprise two VHHs and comprise an amino acid sequence that is at least 97% identical to SEQ ID NO: 83. In some aspects, the first VEGF binding domain and the second VEGF binding domain each comprise two VHHs and comprise an amino acid sequence that is at least 98% identical to SEQ ID NO: 83. In some aspects, the first VEGF binding domain and the second VEGF binding domain each comprise two VHHs and comprise an amino acid sequence according to SEQ ID NO: 83.

In some aspects, the third VEGF binding domain and the fourth VEGF binding domain each comprise two VHHs and comprise an amino acid sequence that is at least 90% identical to SEQ ID NO: 82. In some aspects, the third VEGF binding domain and the fourth VEGF binding domain each comprise two VHHs and comprise an amino acid sequence that is at least 95% identical to SEQ ID NO: 82. In some aspects, the third VEGF binding domain and the fourth VEGF binding domain each comprise two VHHs and comprise an amino acid sequence that is at least 97% identical to SEQ ID NO: 82. In some aspects, the third VEGF binding domain and the fourth VEGF binding domain each comprise two VHHs and comprise an amino acid sequence that is at least 98% identical to SEQ ID NO: 82. In some aspects, the third VEGF binding domain and the fourth VEGF binding domain each comprise two VHHs and comprise an amino acid sequence according to SEQ ID NO: 82.

In some aspects, the third VEGF binding domain and the fourth VEGF binding domain each comprise two VHHs and comprise an amino acid sequence that is at least 90% identical to SEQ ID NO: 83. In some aspects, the third VEGF binding domain and the fourth VEGF binding domain each comprise two VHHs and comprise an amino acid sequence that is at least 95% identical to SEQ ID NO: 83. In some aspects, the third VEGF binding domain and the fourth VEGF binding domain each comprise two VHHs and comprise an amino acid sequence that is at least 97% identical to SEQ ID NO: 83. In some aspects, the third VEGF binding domain and the fourth VEGF binding domain each comprise two VHHs and comprise an amino acid sequence that is at least 98% identical to SEQ ID NO: 83. In some aspects, the third VEGF binding domain and the fourth VEGF binding domain each comprise two VHHs and comprise an amino acid sequence according to SEQ ID NO: 83.

### FZD4 and LRP5 Binding Domains

In an aspect of this disclosure, the binding moiety of the FZD binding domain is derived from an antibody, or an antibody fragment that binds specifically to one FZD, e.g., FZD4, and the co-receptor binding domain comprises a binding moiety that is derived from an antibody or antibody fragment that binds to a LPR5. In an aspect of the disclosure the FZD-binding antibodies bind to an extracellular cysteine rich domain (CRD) of the FZD receptor. The antibody that binds FZD may be an antibody that binds the FZD receptor and antagonizes Wnt signaling or inhibits binding of a Wnt ligand to the FZD receptor. The antibody that binds FZD may be an antibody that binds the FZD receptor without antagonizing or inhibiting binding of a Wnt ligand to the FZD receptor. The antibody that binds FZD may be an antibody that binds FZD and enhances Wnt signaling. The antibody that binds the LRP5 co-receptor may be an antibody that binds the LRP5 co-receptor and antagonizes Wnt signaling or inhibits binding of a Wnt ligand to the co-receptor, or the antibody that binds the LRP5 co-receptor may be an antibody that binds the co-receptor without antagonizing Wnt or Norrin signaling or inhibiting binding of a Wnt or Norrin ligand to the co-receptor.

In an aspect of the disclosure, the LRP5 co-receptor binding domain binds to a single epitope on a co-receptor, e.g., an epitope that binds to the Wnt1 (E1-E2) or Wnt3 (E3-E4) interacting domain of LRP5. In an aspect of the disclosure, the LRP5 co-receptor binding domain binds to two epitopes within the co-receptor, e.g., a paratope that binds to the Wnt1 (E1-E2) interacting epitope and a paratope that binds to Wnt3 (E3-E4) epitope of LRP5. In an aspect of the disclosure, the multivalent antibody binding molecule comprises a Fc domain, wherein the Fc domain is the Fc domain of an immunoglobulin or a fragment thereof comprising the CH3 domain. In an aspect of the disclosure, the immunoglobulin is an IgG. In an aspect of the disclosure, the IgG is an IgG1.

In an aspect of the disclosure, the LRP5 binding domain comprises a diabody comprising two peptides each comprising a heavy chain variable domain (VH) that binds to LRP5 linked to a light-chain variable domain (VL) that binds LRP5 wherein the binding domain is formed by pairing of the VH and the VL from one peptide to the VL and VH of the other peptide thereby forming the LRP5 binding domain.

In an aspect of the disclosure, both of the binding domains of the multivalent antibody binding molecules are bivalent and one or both of the bivalent binding domains are bispecific for the FZD4 receptor and/or LRP5 receptor. For example, the binding molecule may comprise an FZD binding domain that is bivalent and monospecific (each binding site binding to the same epitope) and the LRP5 binding domain is bivalent and bispecific, binding to two different epitopes (the Wnt1 (E1-E2) and Wnt3 (E3-E4) sites on LRP5).

The linker between the VH and VL that bind LRP5 is of a length that promotes the pairing of the VH and VL of the first heavy chain monomer with the VL and VH of the second heavy chain monomer thereby forming a LRP5 receptor binding diabody. The FZD-binding Fabs are formed by the pairing of each heavy chain monomer with a light chain monomer such that the VH that binds FZD4 pairs with the VL that binds FZD4 to form a FZD4 binding domain, and the CH1 of each of the heavy chain monomers pairs with a CL1 (Cκ or Cλ) of each of the light chain monomers. In this IgG-Diabody format, the Fabs form the FZD4-binding domain on the N-terminus of the Fc domain and the diabody forms the co-receptor-binding domain on the C-terminus of the Fc domain. The Fabs may be specific for one FZD, e.g., FZD4 or FZD5, or may be pan-specific, binding to more than one FZD, e.g., to FZD4 and/or FZD5, and in some cases more FZD.

In some aspects, the first VH of the first polypeptide interacts with the VL of the second polypeptide to form a domain that binds LRP5; the first VH of the second polypeptide interacts with the VL of the first polypeptide to form a domain that binds LRP5; the second VH of the first polypeptide interacts with the VL of the third polypeptide to form a domain that binds FZD4; and the second VH of the second polypeptide interacts with the VL of the fourth polypeptide to form a domain that binds FZD4.

In some aspects, peptides forming the diabody in the IgG-Diabody format are linked to the C-terminus of the Fc domain via their VH domain in a VH-VL orientation (N-terminus to C-terminus). In other aspects, the peptides forming the diabody are linked to the C-terminus of the Fc domain via their VL domains in a VL-VH orientation (N-terminus to C-terminus).

In some aspects, the heavy chain sequences forming the Fab molecules described herein are linked to the C-terminus of the Fc domain via their VH domain in a VH-CH1 orientation (N-terminus to C-terminus). In other aspects, the peptides forming the Fab molecules described herein are linked to the C-terminus of the Fc domain via their CH1 domains in a CH1-VH orientation (N-terminus to C-terminus). In some aspects, the light chains associate with these domains to form a properly oriented Fab fragment. In some aspects, the FZD4 binding domain comprises a VH comprising SEQ ID NO: 41 and a VL comprising SEQ ID NO: 42.

In some aspects, the CH1 domains forming the Fab molecules described herein may also comprise an upper hinge (SEQ ID NO: 27) or hinge region (SEQ ID NO: 28) at the C-terminus of the CH1 domain. In some aspects, the CH1 domains forming the Fab molecules described herein comprise SEQ ID NO: 46.

### Fc Heterodimerization

The first and second heavy chain monomers dimerize via their Fc regions, or fragments thereof. The Fc regions may dimerize via a knob-in-hole configuration. Methods for dimerizing peptides via a knob-in-hole configuration are described in WO2018/026942, inventors Van Dyk et al., Carter P. (2001) J. Immunol. Methods 248, 7-15; Ridgway et al. (1996) Protein Eng. 9, 617-621; Merchant, et al. (1998) Nat. Biotechnol. 16, 677-681, and; Atwell et al., (1997) J. Mol. Biol. 270, 26-35. The Fc regions may be Merrimack (knob chain: Q347M, Y349F, T350D, T366W and L368M; hole chain: S354I, E357L, T366S, L368A and Y407V), Merchant (knob chain: T366W; hole chain: T336S, L368A and Y407V) or Merchant S: S (Merchant mutations with additional S354C variant in the knob chain and Y349C in the hole chain).

In some aspects, the first polypeptide and the second polypeptide form a heterodimer. In some aspects, the CH3 of the first polypeptide comprises a serine at position 366, an alanine at position 368 and a valine at position 407 and the CH3 of the second polypeptide comprises a tryptophan at position 366; or the CH3 of the second polypeptide comprises a serine at position 366, an alanine at position 368 and a valine at position 407, and the CH3 of the first polypeptide comprises a tryptophan at position 366, and wherein the positions are according to EU numbering. In some aspects, the first polypeptide or the second polypeptide comprises SEQ ID NO: 21, and the other respective polypeptide comprises SEQ ID NO: 22.

In some aspects, the CH3 of the first polypeptide further comprises an isoleucine at position 354, a leucine at position 357, and a serine at position 360, and the CH3 of the second polypeptide further comprises a methionine at position 347, a phenylalanine at position 349, an aspartic acid at position 350, and a methionine a position 368; or the CH3 of the second polypeptide further comprises an isoleucine at position 354, a leucine at position 357, and a serine at position 360, and the CH3 of the first polypeptide further comprises a methionine at position 347, a phenylalanine at position 349, an aspartic acid at position 350, and a methionine a position 368; and wherein the positions are according to EU numbering.

In some aspects, the first polypeptide comprises a cysteine at position 349, and the second polypeptide comprises a cysteine at position 354, or the second polypeptide comprises a cysteine at position 349, and the first polypeptide comprises a cysteine at position 354, and wherein the positions are according to EU numbering. In some aspects, the first polypeptide or the second polypeptide comprises SEQ ID NO: 23 and the other respective polypeptide comprises SEQ ID NO: 24.

### Fc Effector Function

The Fc regions may also contain mutations that alter their effector function, e.g., the Fc region may have attenuated effector functions due to amino acid mutations, e.g., DANG variants, LALAPG variants, and LALAPS variants. The heavy chain constant region domains (e.g., of a human IgG1) can contain one or more modifications to reduce or eliminate efficient interaction with Fc Receptors on immune cells in the body. Such mutation(s) can abrogate its binding to Fc receptors and reduce or abolish antibody-directed cytotoxicity. Such modifications can include altering the L234L235G236G237 residues (EU numbering) to a LAGA mutation, a FEGG mutation, an AAGG mutation, an AAGA mutation, a LALA mutation or a combination thereof.

Other examples include amino acid substitutions in the Fc regions, e.g., the N297G and D265A, N297G (DANG) variants, L234A, L235A, D265S (LALADS), L234A, L235A, P331S (LALAPS), LALAPS Merchant, LALAPS Merchant S-S (Merchant A. M. et al. Nature Biotechnol 1998 vol 16 p 677-681) variants, or L234A, L235A, P329G (LALA-PG) substitutions, see e.g., Lo et al. “Effector Attenuating Substitutions that Maintain Antibody Stability and Reduce Toxicity in Mice. The Journal of Biological Chemistry Vol. 292, No. 9, pp. 3900-3908 Mar. 3, 2017, incorporated herein by reference.

In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain (κ) or a lambda chain (λ).

In some aspects, the Fc domain has reduced effector function. In some aspects, the CH3 of the first polypeptide and/or the CH3 of the second polypeptide comprise a glycine at position 397, an alanine at position 265, or both a glycine at position 397 and an alanine at position 265, wherein the positions according to EU numbering. In some aspects, the CH3 of the first polypeptide and/or the CH3 of the second polypeptide comprise a glycine at position 397 and an alanine at position 265, wherein the positions according to EU numbering. In some aspects, the CH2 of the first polypeptide and/or the CH2 of the second polypeptide comprise an alanine at position 234, an alanine at position 235, a serine at position 331, or any combination thereof, wherein the positions are according to EU numbering. In some aspects, the CH2 of the first polypeptide and/or the CH2 of the second polypeptide comprise an alanine at position 234 and an alanine at position 235, wherein the positions are according to EU numbering. In some aspects, the CH2 of the first polypeptide and the CH2 of the second polypeptide comprise an alanine at position 234, an alanine at position 235, and a serine a position 331, wherein the positions are according to EU numbering. In some aspects, the first polypeptide and/or the second polypeptide comprises SEQ ID NO: 18. In some aspects, the first polypeptide and/or the second polypeptide comprises SEQ ID NO: 19. In some aspects, the first polypeptide or the second polypeptide comprises SEQ ID NO: 18 and the other respective polypeptide comprises SEQ ID NO: 19.

### Peptide Linkers

The various domains of the multivalent molecules described herein, including domains such as VL, VH, CH1, CH2, CH3, CL1 and Fc, may be joined via linkers, e.g., peptide linkers. The binding domains of the FZD Agonists may be linked to the Fc domain via a linker. In some aspects, adjacent VH and VL domains are attached to each other via a peptide linker. In some aspects adjacent constant domains and variable domains are attached via a peptide linker. The linker may be, e.g., a polypeptide linker, or a non-peptidic linker. In some aspects the constant domains and variable domains of the FZD Agonists are attached to the Fc domain via a peptide linker. G4S linkers can also be used herein. These linkers are a type of flexible, unstructured synthetic peptide linker sequence, typically used to connect the VH and VL domain of a variety of antigen binding formats, including diabodies and single-chain variable fragments (scFvs), or other sequences.

In some aspects, the linker is an upper hinge (SEQ ID NO: 27) or hinge region (SEQ ID NO: 28). In some aspects, the peptide linker is a G4S linker. In some aspects, the peptide linker contains one or more G4S repeats (GGGGS, SEQ ID NO: 29). In some aspects, the linker comprises from one to about 10 G4S repeats. In some aspects, the linker comprises two G4S repeats. In some aspects, the linker comprises three G4S repeats. In some aspects, the linker comprises four G4S repeats. In some aspects, the linker comprises any of the linker sequences found in SEQ ID NOs: 29-33. In some aspects, the linker comprises any of the linker sequences found in SEQ ID NOs: 29-33, and either SEQ ID NO: 27 or SEQ ID NO: 28. In some aspects, the linker comprises SEQ ID NO: 32 and SEQ ID NO: 28. In some aspects, the peptide linker is a G4A linker. In some aspects, the peptide linker contains one or more G4A repeats (GGGGA, SEQ ID NO: 117). In some aspects, the linker comprises from one to about 10 G4A repeats. In some aspects, the linker comprises two G4A repeats. In some aspects, the linker comprises three G4A repeats. In some aspects, the linker comprises four G4A repeats. In some aspects, the linker comprises SEQ ID NO: 117. In some aspects, the linker comprises SEQ ID NO: 118. In some aspects, the linker comprises SEQ ID NO: 119.

The Fc domain of the FZD Agonists, with or without the linker, is of a length and flexibility that allows for the multivalent antibody binding molecule of this disclosure to bind both the FZD receptor and its LRP5 receptor thereby stabilizing receptor conformations that are compatible with activation of downstream Wnt signaling pathways. In an aspect of this disclosure the Fc domain, or fragment thereof comprising the CH3 domain, with or without the linker is greater than 100 amino acids spanning up to 300 Å, greater than 125 amino acids spanning up to 375 Å, greater than 150 amino acids spanning up to 450 Å, greater than 175 amino acids spanning up to 525 Å, or greater than 300 amino acids spanning up to 900 Å. Preferably the Fc domain is about 200 amino acids to about 300 amino acids in length.

In some aspects, the VL of the first polypeptide is attached to the CH2 of the first polypeptide by a first polypeptide linker, and the VL of the second polypeptide is attached to CH2 of the second polypeptide by a second polypeptide linker.

In some aspects, the second VH of the first polypeptide is attached to the CH3 of the first polypeptide by a third polypeptide linker, and the second VH of the second polypeptide is attached to CH3 of the second polypeptide by a fourth polypeptide linker.

In some aspects, the first polypeptide linker and/or the second polypeptide linker comprise or consist of the amino acid sequence of GGGGSGGGGSEPKSS (SEQ ID NO: 32). In some aspects, the first polypeptide linker and/or the second polypeptide linker comprise or consist of the amino acid sequence of GGGGSGGGGSEPKSSDKTHT (SEQ ID NO: 33). In some aspects, the first polypeptide linker and the second polypeptide linker comprise or consist of the amino acid sequence of GGGGSGGGGSEPKSSDKTHT (SEQ ID NO: 33). In some aspects, the third polypeptide linker and/or the fourth polypeptide linker comprise or consist of the amino acid sequence of GGGSGGGSGGGSGGGSGSTG (SEQ ID NO: 34). In some aspects, the third polypeptide linker and the fourth polypeptide linker comprise or consist of the amino acid sequence of GGGSGGGSGGGSGGGSGSTG (SEQ ID NO: 34).

In some aspects, the first VH of the first polypeptide is linked to the VL of the first polypeptide by a fifth polypeptide linker, and the first VH of the second polypeptide is linked to the VL of the second polypeptide by a sixth polypeptide linker.

In some aspects, the fifth polypeptide linker comprises or consists of the amino acid sequence of GGGGS (SEQ ID NO: 29); and the sixth polypeptide linker comprises or consists of the amino acid sequence of GGGGS (SEQ ID NO: 29).

In some aspects, the first VEGF binding domain is attached to the second VH of the first polypeptide by a seventh polypeptide linker, and/or the second VEGF binding domain is attached to the second VH of the second polypeptide by an eighth polypeptide linker. In some aspects, the seventh polypeptide linker and/or the eighth polypeptide linker comprise or consist of the amino acid sequence of GGGGSGGGGSEPKSS (SEQ ID NO: 32). In some aspects, the seventh polypeptide linker and/or the eighth polypeptide linker comprise or consist of the amino acid sequence of GGGGSGGGGSEPKSSDKTHT (SEQ ID NO: 33). In some aspects, the seventh polypeptide linker and the eighth polypeptide linker comprise or consist of the amino acid sequence of GGGGSGGGGSEPKSSDKTHT (SEQ ID NO: 33).

In some aspects, the seventh polypeptide linker comprises or consists of the amino acid sequence of GGGGS (SEQ ID NO: 29); and the eighth polypeptide linker comprises or consists of the amino acid sequence of GGGGS (SEQ ID NO: 29). In some aspects, the seventh polypeptide linker comprises or consists of the amino acid sequence of GGGGA (SEQ ID NO: 117); and the eighth polypeptide linker comprises or consists of the amino acid sequence of GGGGA (SEQ ID NO: 117). In some aspects, the seventh polypeptide linker comprises one or more repeats of the amino acid sequence of GGGGA (SEQ ID NO: 117); and the eighth polypeptide linker comprises one or more repeats of the amino acid sequence of GGGGA (SEQ ID NO: 117). In some aspects, the seventh polypeptide linker comprises or consists of the amino acid sequence of SEQ ID NO: 118, and the eighth polypeptide linker comprises or consists of the amino acid sequence of SEQ ID NO: 118.

### Variable Domains and Sequences

Without wishing to be bound by theory, it is contemplated that the multispecific antibody binding molecules of this disclosure facilitate the interaction of a FZD receptor and an LRP5 receptor on a cell by promoting their proximity and stabilizing conformations of the receptor proteins that are favorable for activating Wnt signaling pathways. Another aspect of this disclosure is a method for facilitating the interaction of a FZD receptor and an LRP5 receptor on a cell thereby activating a Wnt signaling pathway in the cell. The Wnt co-receptor binding domain and FZD binding domain are bivalent and each comprise a VL and/or a VH, and one or both of the binding domains may be monospecific. In an aspect of the disclosure one or both the Wnt co-receptor binding domain and FZD binding domain are bispecific. In an aspect of the disclosure the Wnt co-receptor binding domain is bivalent and bispecific. The FZD binding domain may comprise a scFv that binds FZD, or a Fab that binds FZD, or combinations thereof, or a diabody that binds FZD. The Wnt co-receptor binding domain may comprise a scFv that binds the LRP5 co-receptor, a Fab that binds the LRP5 receptor, or combinations thereof, or a diabody that binds the LRP5 receptor. In an aspect of the disclosure the FZD binding domain comprises two FZD-binding Fabs and the Wnt co-receptor binding domain comprises a bispecific bivalent diabody that binds LRP5 on two different epitopes.

The VH and VL domains of the FZD binding domain of the multivalent molecules of this disclosure may comprise the three light chain CDRs and three heavy chain CDRs of a FZD source antibody, e.g., the FZD4 binding antibodies of Table 2, or three light chain CDRs and three heavy chain CDRs according to Table 8 and VH and VL sequences that are at least 50%, at least 55%, at least 60%, at least 75, at least 80%, at least 85%, at least 90%, at least 92%, at least 95%, at least 96%, at least 97%, at least 98% or at least 99% identical to the VH and VL sequences of the FZD4 antibody of Table 8.

In some aspects, FZD4 binding domains of the multivalent molecules of the disclosure comprise a VH that is at least 90% identical to SEQ ID NO: 41, and a VL that is at least 90% identical to SEQ ID NO: 42. In some aspects, FZD4 binding domains of the multivalent molecules of this disclosure comprise a VH that is at least 95% identical to SEQ ID NO: 41, and a VL that is at least 95% identical to SEQ ID NO: 42. In some aspects, FZD4 binding domains of the multivalent molecules of this disclosure comprise a VH that is at least 98% identical to SEQ ID NO: 41, and a VL that is at least 98% identical to SEQ ID NO: 42. In some aspects, FZD4 binding domains of the multivalent molecules of this disclosure comprise a VH according to SEQ ID NO: 41, and a VL according to SEQ ID NO: 42.

The VH and VL domains of the LRP5 receptor binding domain of the multivalent molecules of this disclosure may comprise the three light chain CDRs and three heavy chain CDRs of an LRP5 receptor source antibody, e.g., the LRP5 binding antibodies of Table 8, or three light chain CDRs and three heavy chain CDRs according to Table 8 and VH and VL sequences that are at least 50%, at least 55%, at least 60%, at least 75%, at least 80%, at least 85%, at least 90%, at least 95%, at least 96%, at least 97%, at least 98% or at least 99% identical to the VH and VL of the Wnt co-receptor source antibody, e.g., the LRP5 binding antibodies of Table 2.

In some aspects, the LRP5 receptor binding domain of the multivalent molecules of the disclosure comprise a VH that is at least 90% identical to SEQ ID NO: 13, and a VL that is at least 90% identical to SEQ ID NO: 14. In some aspects, LRP5 receptor binding domain of the multivalent molecules of this disclosure comprise a VH that is at least 90% identical to SEQ ID NO: 15, and a VL that is at least 90% identical to SEQ ID NO: 16. In some aspects, LRP5 receptor binding domain of the multivalent molecules of this disclosure comprise a VH that is at least 95% identical to SEQ ID NO: 13, and a VL that is at least 95% identical to SEQ ID NO: 14. In some aspects, LRP5 receptor binding domain of the multivalent molecules of this disclosure comprise a VH that is at least 95% identical to SEQ ID NO: 15, and a VL that is at least 95% identical to SEQ ID NO: 16. In some aspects, LRP5 receptor binding domain of the multivalent molecules of this disclosure comprise a VH that is at least 98% identical to SEQ ID NO: 13, and a VL that is at least 98% identical to SEQ ID NO: 14. In some aspects, LRP5 receptor binding domain of the multivalent molecules of this disclosure comprise a VH that is at least 98% identical to SEQ ID NO: 15, and a VL that is at least 98% identical to SEQ ID NO: 16. In some aspects, LRP5 receptor binding domain of the multivalent molecules of this disclosure comprise a VH according to SEQ ID NO: 13, and a VL according to SEQ ID NO: 14. In some aspects, LRP5 receptor binding domain of the multivalent molecules of this disclosure comprise a VH according to SEQ ID NO: 15, and a VL according to SEQ ID NO: 16.

In an aspect of the disclosure is a polypeptide comprising the first heavy chain monomer (i.e., the first polypeptide) of the binding antibody molecules of the disclosure. In a further aspect, the polypeptide comprises a sequence which has at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to any sequence selected from SEQ ID NOs: 59, 62, 65, 68, 71, 74, or 77. In a further aspect, the polypeptide comprises a sequence that has about 70% identity, about 75% identity, about 80% identity, about 85% identity, about 90% identity, about 91% identity, about 92% identity, about 93% identity, about 94% identity, about 95% identity, about 96% identity, about 97% identity, about 98% identity, or about 99% identity, to any sequence selected from SEQ ID NOs: 59, 62, 65, 68, 71, 74, or 77. In a further aspect, the polypeptide comprises the sequence set forth in any of SEQ ID NOs: 59, 62, 65, 68, 71, 74, or 77.

In an aspect of the disclosure is a polypeptide comprising the second heavy chain monomer (i.e., the second polypeptide) of the binding antibody molecules of the disclosure. In a further aspect, the polypeptide comprises a sequence that has at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to any sequence selected from SEQ ID NOs: 60, 63, 66, 69, 72, 75, or 78. In a further aspect, the polypeptide comprises a sequence that has about 70% identity, about 75% identity, about 80% identity, about 85% identity, about 90% identity, about 91% identity, about 92% identity, about 93% identity, about 94% identity, about 95% identity, about 96% identity, about 97% identity, about 98% identity, or about 99% identity, to any sequence selected from SEQ ID NOs: 60, 63, 66, 69, 72, 75, or 78. In a further aspect, the polypeptide comprises the sequence set forth in any of SEQ ID NOs: 60, 63, 66, 69, 72, 75, or 78.

In an aspect of the disclosure is a polypeptide comprising a light chain monomer (i.e., the third polypeptide and/or the fourth polypeptide) of the binding antibody molecules of the disclosure. In a further aspect, the polypeptide comprises a sequence which has at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 61, 64, 67, 70, 73, 76, or 79. In a further aspect, the polypeptide comprises a sequence that has about 70% identity, about 75% identity, about 80% identity, about 85% identity, about 90% identity, about 91% identity, about 92% identity, about 93% identity, about 94% identity, about 95% identity, about 96% identity, about 97% identity, about 98% identity, or about 99% identity, to any sequence selected from SEQ ID NOs: 61, 64, 67, 70, 73, 76, or 79. In a further aspect, the polypeptide comprises the sequence set forth in any of SEQ ID NOs: 61, 64, 67, 70, 73, 76, or 79. 59. In some aspects, the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 64.

The present disclosure is also directed to a multivalent antibody binding molecule, comprising: (a) a first polypeptide comprising the amino acid sequence of SEQ ID NO: 59; (b) a second polypeptide comprising the amino acid sequence of SEQ ID NO: 60; (c) a third polypeptide comprising the amino acid sequence of SEQ ID NO: 61; (d) a fourth polypeptide comprising the amino acid sequence of SEQ ID NO: 61; and (c) one or more VEGF binding domains that each specifically bind vascular endothelial growth factor (VEGF); wherein each VEGF binding domain comprises the amino acid sequence of SEQ ID NO: 53 or SEQ ID NO: 57.

In an aspect of the disclosure is a multivalent antibody binding molecule comprises (a) in the first polypeptide, the VH that binds LRP5 comprises CDR-H1 of SEQ ID NO: 1, CDR-H2 of SEQ ID NO: 2 and CDR-H3 of SEQ ID NO: 3 and the VL that binds LRP5 comprises CDR-L1 of SEQ ID NO: 10, CDR-L2 of SEQ ID NO: 11 and CDR-L3 of SEQ ID NO: 12; and the VH that binds FZD4 comprises the FZD4 VH CDRs CDR-H1 of SEQ ID NO: 35, a CDR-H2 of SEQ ID NO: 36 and a CDR-H3 of SEQ ID NO: 37, and (b) in the second polypeptide, the VH that binds LRP5 comprises CDR-H1 of SEQ ID NO: 7, CDR-H2 of SEQ ID NO: 8 and CDR-H3 of SEQ ID NO: 9 and the VL that binds LRP5 comprises CDR-L1 of SEQ ID NO: 4, CDR-L2 of SEQ ID NO: 5 and CDR-L3 of SEQ ID NO: 6, and the VH that binds FZD4 comprises the FZD4 VH CDRs CDR-H1 of SEQ ID NO: 35, a CDR-H2 of SEQ ID NO: 36 and a CDR-H3 of SEQ ID NO: 37, and (c) in each of the third polypeptide and fourth polypeptide, the VL that binds FZD4 comprises the CDR-L1 of SEQ ID NO: 38, CDR-L2 of SEQ ID NO: 39 and CDR-L3 of SEQ ID NO: 40.

In some aspects, the disclosure is directed to a multivalent antibody binding molecule, comprising an LRP5 binding domain that specifically binds low-density lipoprotein receptor-related protein 5 (LRP5), an Fc domain, an FZD4 binding domain that specifically binds Frizzled 4 (FZD4), and one or more VEGF binding domains that each specifically bind vascular endothelial growth factor (VEGF); wherein the multivalent antibody molecule comprises four polypeptides, wherein the first polypeptide comprises a first VH comprising a CDR-H1 having the amino acid sequence of SEQ ID NO: 1, a CDR-H2 having the amino acid sequence of SEQ ID NO: 2, and a CDR-H3 having the amino acid sequence of SEQ ID NO: 3; a VL comprising a CDR-L1 having the amino acid sequence of SEQ ID NO: 10, a CDR-L2 having the amino acid sequence of SEQ ID NO: 11, and a CDR-L3 having the amino acid sequence of SEQ ID NO: 12; and a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37; the second polypeptide comprises: a first VH comprising a CDR-H1 having the amino acid sequence of SEQ ID NO: 7, a CDR-H2 having the amino acid sequence of SEQ ID NO: 8, and a CDR-H3 having the amino acid sequence of SEQ ID NO: 9; a VL comprising a CDR-L1 having the amino acid sequence of SEQ ID NO: 4, a CDR-L2 having the amino acid sequence of SEQ ID NO: 5, and a CDR-L3 having the amino acid sequence of SEQ ID NO: 6; and a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37; and the third polypeptide and the fourth polypeptide comprise a VL comprising a CDR-L1 having the amino acid sequence of SEQ ID NO: 38, a CDR-L2 having the amino acid sequence of SEQ ID NO: 39, and a CDR-L3 having the amino acid sequence of SEQ ID NO: 40, and wherein each of the VEGF binding domains comprise a VEGF receptor component.

In an aspect of the disclosure the first polypeptide a VH that binds LRP5 comprising CDR-H1 of SEQ ID NO: 1, CDR-H2 of SEQ ID NO: 2 and CDR-H3 of SEQ ID NO: 3; a VL that binds LRP5 comprising CDR-L1 of SEQ ID NO: 10, CDR-L2: of SEQ ID NO: 11 and CDR-L3 of SEQ ID NO: 12; and a VH that binds FZD4 comprising CDR-H1 of SEQ ID NO: 35, CDR-H2 of SEQ ID NO: 36 and CDR-H3 of SEQ ID NO: 37; wherein the first polypeptide and/or the second polypeptide comprises a sequence which has at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to any sequence selected from SEQ ID NOs: 13, 15, 59, 60, 62, 63, 65, 66, 68, 69, 71, 72, 74, 75, 77, or 78. In a further aspect, the polypeptide comprises a sequence that has about 70% identity, about 75% identity, about 80% identity, about 85% identity, about 90% identity, about 91% identity, about 92% identity, about 93% identity, about 94% identity, about 95% identity, about 96% identity, about 97% identity, about 98% identity, or about 99% identity to any sequence selected from SEQ ID NOs: 13, 15, 59, 60, 62, 63, 65, 66, 68, 69, 71, 72, 74, 75, 77, or 78. In a further aspect, the polypeptide comprises the sequence set forth in any of SEQ ID NOs: 13, 15, 59, 60, 62, 63, 65, 66, 68, 69, 71, 72, 74, 75, 77, or 78.

In an aspect of the disclosure the third polypeptide and fourth polypeptide comprise a VL that binds FZD4 comprising CDR-L1 of SEQ ID NO: 38, CDR-L2 of SEQ ID NO: 39 and CDR-L3 of SEQ ID NO: 40, wherein the third polypeptide and the fourth polypeptide comprise a sequence which has at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NOs: 14, 16, 42, 61, 64, 67, 70, 73, 76, 79, 80, or 81. In a further aspect, the polypeptide comprises a sequence that has about 70% identity, about 75% identity, about 80% identity, about 85% identity, about 90% identity, about 91% identity, about 92% identity, about 93% identity, about 94% identity, about 95% identity, about 96% identity, about 97% identity, about 98% identity, or about 99% identity to any sequence selected from SEQ ID NOs: 14, 16, 42, 61, 64, 67, 70, 73, 76, 79, 80, or 81. In a further aspect, the polypeptide comprises the sequence set forth in any of SEQ ID NOs: 14, 16, 42, 61, 64, 67, 70, 73, 76, 79, 80, or 81.

In some aspects, the first polypeptide comprises an amino acid sequence of any one of SEQ ID NOs: 59, 62, 65, 68, 71, 74, or 77, the second polypeptide comprises the amino acid of any one of SEQ ID NOs: 60, 63, 66, 69, 72, 75, or 78, and the third polypeptide and the fourth polypeptide each comprise an amino acid sequence of any one of SEQ ID NOs: 61, 64, 67, 70, 73, 76, or 79. In some aspects, the first polypeptide comprises an amino acid sequence of any one of SEQ ID NOs: 87, 90, 93, 96, 99, 102, 105, 108, 111, or 114, the second polypeptide comprises the amino acid sequence of any one of SEQ ID NOs: 88, 91, 94, 97, 100, 103, 106, 109, 112, or 115, and the third polypeptide and the fourth polypeptide each comprise an amino acid sequence of any one of SEQ ID NOs: 89, 92, 95, 98, 101, 104, 107, 110, 113, or 116.

In some aspects, the first polypeptide comprises an amino acid sequence of any one of SEQ ID NO: 59, 62, 65, 68, 71, 74, or 77, the second polypeptide comprises the amino acid of any one of SEQ ID NO: 60, 63, 66, 69, 72, 75, or 78, and the third polypeptide and the fourth polypeptide each comprise an amino acid sequence of any one of SEQ ID NO: 80 or 81.

The present disclosure is also directed to a multivalent antibody binding molecule comprising a first polypeptide, a second polypeptide, a third polypeptide, and a fourth polypeptide. In some aspects, the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 61. In some aspects, the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 80. In some aspects, the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 81. In some aspects, the first polypeptide comprises the amino acid sequence of SEQ ID NO: 87, the second polypeptide comprises the amino acid of SEQ ID NO: 88, and the third polypeptide and the fourth polypeptide each comprise the amino acid sequence of SEQ ID NO: 89. In some aspects, the first polypeptide comprises the amino acid sequence of SEQ ID NO: 90, the second polypeptide comprises the amino acid of SEQ ID NO: 91, and the third polypeptide and the fourth polypeptide each comprise the amino acid sequence of SEQ ID NO: 92. In some aspects, the first polypeptide comprises the amino acid sequence of SEQ ID NO: 93, the second polypeptide comprises the amino acid of SEQ ID NO: 94, and the third polypeptide and the fourth polypeptide each comprise the amino acid sequence of SEQ ID NO: 95. In some aspects, the first polypeptide comprises the amino acid sequence of SEQ ID NO: 96, the second polypeptide comprises the amino acid of SEQ ID NO: 97, and the third polypeptide and the fourth polypeptide each comprise the amino acid sequence of SEQ ID NO: 98. In some aspects, the first polypeptide comprises the amino acid sequence of SEQ ID NO: 99, the second polypeptide comprises the amino acid of SEQ ID NO: 100, and the third polypeptide and the fourth polypeptide each comprise the amino acid sequence of SEQ ID NO: 101. In some aspects, the first polypeptide comprises the amino acid sequence of SEQ ID NO: 99, the second polypeptide comprises the amino acid of SEQ ID NO: 100, and the third polypeptide and the fourth polypeptide each comprise the amino acid sequence of SEQ ID NO: 101.

In some aspects, the first polypeptide comprises the amino acid sequence of SEQ ID NO: 102, the second polypeptide comprises the amino acid of SEQ ID NO: 103, and the third polypeptide and the fourth polypeptide each comprise the amino acid sequence of SEQ ID NO: 104. In some aspects, the first polypeptide comprises the amino acid sequence of SEQ ID NO: 105, the second polypeptide comprises the amino acid of SEQ ID NO: 106, and the third polypeptide and the fourth polypeptide each comprise the amino acid sequence of SEQ ID NO: 107. In some aspects, the first polypeptide comprises the amino acid sequence of SEQ ID NO: 108, the second polypeptide comprises the amino acid of SEQ ID NO: 109, and the third polypeptide and the fourth polypeptide each comprise the amino acid sequence of SEQ ID NO: 110. In some aspects, the first polypeptide comprises the amino acid sequence of SEQ ID NO: 111, the second polypeptide comprises the amino acid of SEQ ID NO: 112, and the third polypeptide and the fourth polypeptide each comprise the amino acid sequence of SEQ ID NO: 113. In some aspects, the first polypeptide comprises the amino acid sequence of SEQ ID NO: 114, the second polypeptide comprises the amino acid of SEQ ID NO: 115, and the third polypeptide and the fourth polypeptide each comprise the amino acid sequence of SEQ ID NO: 116.

In an aspect of the disclosure the polypeptides comprising monomer chains further comprise a signal peptide. In some aspects, the signal peptide is present at the N-terminus of the first polypeptide, the N-terminus of the second polypeptide, the N-terminus of the third polypeptide, and/or the N-terminus of the fourth polypeptide. In some aspects, the signal peptide comprises SEQ ID NO: 58. In some aspects, the signal peptide comprises SEQ ID NO: 84. In some aspects, the signal peptide comprises SEQ ID NO: 85. In some aspects, the signal peptide comprises SEQ ID NO: 86. In some aspects, the signal peptide for the first polypeptide and/or the second polypeptide is SEQ ID NO: 84. In some aspects, the signal peptide for the first polypeptide and/or the second polypeptide is SEQ ID NO: 86. In some aspects, the signal peptide for the third polypeptide and/or the fourth polypeptide is SEQ ID NO: 85. In some aspects, the signal peptide for the first polypeptide and the second polypeptide is SEQ ID NO: 84, and the signal peptide for the third polypeptide and the fourth polypeptide is SEQ ID NO: 85. In some aspects, the signal peptide for the first polypeptide and the second polypeptide is SEQ ID NO: 86, and the signal peptide for the third polypeptide and the fourth polypeptide is SEQ ID NO: 85. In some aspects, the signal peptide for the first polypeptide and the second polypeptide is SEQ ID NO: 86, and the signal peptide for the third polypeptide and the fourth polypeptide is SEQ ID NO: 84. In an aspect of the disclosure, the polypeptides comprising monomer chains do not comprise a signal peptide. The signal peptide may be cleaved from the immature chain to produce the mature chain.

### Uses and Methods

The compositions described herein may be administered to a patient in need thereof to regulate angiogenesis, endothelial signaling, inflammation, and/or vascular leakage. Angiogenesis means the formation or growth of new blood vessels from previously existing blood vessels. Examples of angiogenic or angiogenesis-related diseases include, but are not limited to diabetic retinopathy, diabetic macular edema, retinopathy of prematurity, corneal graft rejection, macular degeneration, glaucoma such as neovascular glaucoma, systemic erythrosis, proliferative retinopathy, psoriasis, hemophilic arthritis, or capillary formation in atherosclerotic plaques.

In some aspects, the disclosure provides a method for treating an ocular disorder in a patient in need thereof comprising administering an effective amount of a multivalent antibody binding molecule as disclosed herein to the patient. In some aspects, the disclosure provides a method for treating macular degeneration in a patient in need thereof comprising administering an effective amount of a multivalent antibody binding molecule as disclosed herein to the patient. In some aspects, the disclosure provides a method for treating diabetic macular edema in a patient in need thereof comprising administering an effective amount of a multivalent antibody binding molecule as disclosed herein to the patient.

The methods of the disclosure include procedures for administering a pharmaceutical composition of pharmaceutically effective dose for individuals in need of inhibition of angiogenesis. The individual can be a mammal such as dog, cat, ferret, cow, horse, rabbit, mouse, rat, or human, but it is not limited thereto. For example, in one aspect, the individual can be a chicken, turkey, or other non-mammalian animal. The pharmaceutical composition can be administered via a suitable way including parenterally, subcutaneously, intraperitoneally, intrapulmonarily or intranasally, and if necessary, intralesionally for local treatment. In some aspects, the dosage of the pharmaceutical composition changes depending on various factors including, but not limited to, the health status and weight of the individual, the severity of the disease, the type of drug, the route and time of administration, and it can be easily determined by those skilled in the art.

The multivalent antibody binding molecule, and compositions thereof, may be administered to mammals, including rats, mice, livestock, companion animals, humans, etc. In some aspects, the subject is human. In some aspects, the subject is a companion animal, such as a mammalian companion animal. In some aspects, the companion animal is a dog, cat, rabbit, ferret, horse, mule, donkey, or hamster. In some aspects, administration is by intravitreal injection. In another aspect, the present disclosure relates to methods and compositions for preventing or treating angiogenic or vascular diseases by administering an effective amount of the multivalent antibody binding molecule. In other aspects, the invention relates to methods and compositions for regulating angiogenesis, endothelial signaling, inflammation, and/or vascular leakage by administering the multivalent antibody binding molecule to a subject in need thereof. In some aspects, an antibody binding molecule described herein is for use in therapy.

In some aspects, the ocular disorder is selected from diabetic retinopathy, retinopathy of prematurity, Coats' disease, Familial Exudative Vitreoretinopathy (FEVR), Norrie disease, macular degeneration, diabetic macular edema, and pediatric vitreoretinopathies. In some aspects, the ocular disorder is macular degeneration. In some aspects, the ocular disorder is diabetic macular edema. In some aspects, the macular degeneration is neovascular age-related macular degeneration (nvAMD). In aspects of any of the methods described herein, the multivalent antibody binding molecules, or compositions thereof, are administered to a subject through intravitreal injection.

Aspects of the invention also include one or more of the multivalent antibody binding molecules, and compositions thereof as described herein (i) for use in, (ii) for use as a medicament or composition for, or (iii) for use in the preparation of a medicament for: (a) therapy (e.g., of the human body); (b) medicine; (c) treatment of an ocular disorder; (d) regulation of angiogenesis; (c) regulation of endothelial signaling; (f) regulation of inflammation, and/or vascular leakage; (g) treatment of diabetic macular edema; (h) treatment of macular degeneration; (i) treatment of nvAMD, (j) treatment of diabetic retinopathy; (k) treatment of retinopathy of prematurity; (l) treatment of Coats' disease; (m) treatment of Familial Exudative Vitreoretinopathy (FEVR); (n) treatment of Norrie disease; (o) treatment of pediatric vitreoretinopathies; or (p) reduction of the progression, onset or severity of pathological symptoms associated any disease or condition described in one or more of (g)-(o).

### Pharmaceutical Compositions

The antibody molecule may be formulated with a suitable vehicle, excipient or diluent typically used in the field. In some aspects, the composition comprising the multivalent antibody binding molecule is a pharmaceutical composition. The disclosure provides, in some aspects, a pharmaceutical composition comprising any one of the antibody binding molecules described herein and a pharmaceutically acceptable carrier. In other aspects, the disclosure provides a pharmaceutical composition comprising one or more of the antibody binding molecules described herein and a pharmaceutically acceptable carrier.

Suitable vehicles, excipients, diluents are described in Remington: The Science and Practice of Pharmacy (19th ed.) ed. A. R. Gennaro, Mack Publishing Company, Easton, Pa. 1995. Suitable pharmaceutically acceptable vehicles, excipients or diluents include, for example, one or more of water, saline, phosphate buffered saline, dextrose, histidine, glycerol, sucrose, polysorbate, ethanol and the like, as well as combinations thereof. In some embodiments, the pH of the solution is from about 5 to about 8. In other embodiments, the pH of the solution is from about 6 to about 7. It will be apparent to those persons skilled in the art that certain carriers may be more preferable depending upon, for instance, the route of administration and concentration.

Pharmaceutical compositions having a pharmaceutically acceptable vehicle can be various oral or parenteral dosage form such as tablets, pills, powders, granules, capsules, suspensions, oral solutions, emulsions, syrups, sterile aqueous solutions, non-aqueous solutions, suspensions, lyophilizates, and suppository. The pharmaceutical composition can include a diluent or excipient, which can be formulated in combination, such as fillers, thickeners, binders, wetting agents, disintegrants, surfactants, etc. Solid preparations for oral administration may be in the form of tablets, pills, powders, granules, capsules, and the like. Simple excipient and lubricating agents may additionally be used.

The liquid preparation for oral administration may be a suspension, an oral solution, an emulsion, a syrup, or the like. Excipients such as water or simple diluents like wet paraffin, a variety of wetting agents, sweeteners, aromatics, preservatives, and etc. can be included in a liquid formulation. In addition, the pharmaceutical compositions may be in parenteral dosage form such as sterile aqueous solution, non-aqueous solvent, suspension, emulsion, lyophilizate, suppository, etc. Injectable propylene glycol, polyethylene glycol, vegetable oils such as olive oil and esters such as ethyl oleate may be suitable for insoluble solvent and suspension. The basic substance of the suppository includes Witepsol, macrogol, Tween 61, cacao butter, laurin butter and glycerogelatin.

The composition is administered in a pharmaceutically effective amount. Terms used herein, such as a “pharmaceutically effective amount” or “therapeutically effective amount” refer to an amount of the pharmaceutical composition effective for disease treatment, with an appropriate benefit/risk ratio which can be applicable for all medical treatments. The effective amount can be different depending on various factors including parameters like the severity of the disease, the patient's age and sex, type of disease, drug activity, drug sensitivity, administration time, route of administration, secretion rate, duration of treatment, and other factors. Also, the above composition can be administered in single doses or divided into multiple doses. When fully considering these factors, it is important to administer the minimum amount sufficient to obtain maximum effect without side effects. The dosage of the pharmaceutical composition is not particularly limited, but it varies depending on various factors, including patient's health status and weight, disease severity, drug type, administration route and administration time.

The composition may be administered into mammals, including rats, mice, livestock, companion animals, humans, etc., via typically accepted routes, for example, orally, rectal, intravenously, subcutaneously, intrauterinely, intravitreally, or intracerebrovascularly. In some aspects, the composition is administered by intravitreal injection. In some aspects, the subject is human. In some aspects, the subject is a companion animal, such as a mammalian companion animal. In some aspects, the companion animal is a dog, cat, rabbit, ferret, horse, mule, donkey, hamster, or other domesticated pet.

The present disclosure also relates to prevention or treatment methods for angiogenic or vascular diseases, and anti-angiogenic methods, including the steps for administering into an individual in need of the antibody or the above composition.

The multivalent antibody binding molecule can be provided in a pharmaceutical composition.

### Nucleic Acids and Expression

In some aspects, the disclosure relates to a nucleic acid encoding any of the multivalent antibody binding molecules disclosed herein. In other aspects, the disclosure relates to a nucleic acid encoding any one of the polypeptides included within a multivalent antibody binding molecule disclosed herein. The DNA encoding the multivalent antibody binding molecule is easily separated or synthesized using a conventional process (for example, by using oligonucleotide probes capable of specifically binding to the DNA encoding the heavy and light chains) Many vectors are available. Vector components generally include one or more of the following, but is not limited to: signal sequence, origin of replication, one or more marker genes, enhancer element, promoter, and transcription termination sequence.

In some aspects, the disclosure provides an expression vector comprising the nucleic acid encoding the multivalent antibody binding molecule. In another aspect, there is provided a vector which comprises nucleic acids encoding one heavy chain sequence and one light chain sequence of the multivalent antibody binding molecule.

In one aspect, there is provided a set of one or more vectors which collectively comprise the set of one or more polynucleotides of the disclosure, such that all chains (i.e., all light and heavy chains) of said antibody binding molecule are encoded in the set of vectors.

In one aspect, the vector is an animal virus, such as a virus selected from reverse transcriptase virus (including lentivirus), adenovirus, adeno-associated virus, herpes virus, varicella-zoster virus, baculovirus, papillomavirus, and papovavirus.

In some aspects, the expression vector further comprises a promoter/enhancer, such as a human cytomegalovirus IE1 (CMV-IE1) promoter/enhancer.

In some aspects, provided herein is a host cell comprising a polynucleotide encoding an antibody binding molecule described herein. In some aspects, provided herein is a host cell comprising a vector comprising a polynucleotide encoding an antibody binding molecule described herein. The host cell may be prokaryotic or eukaryotic. The host cell may be isolated, e.g., cultured or not part of a multicellular organism. In some aspects, the host cell is a member of a cell line. In some aspects, the host cell is a mammalian cell. In some aspects, the host cell is an immortalized mammalian cell.

In the case of prokaryotic cell as a host, a strong promotor which can process the transcription (for example, tac promoter, lac promoter, lacUV5 promoter, 1 pp promoter, pLλ promoter, pRλ promoter, rac5 promoter, amp promoter, recA promoter, SP6 Promoter, trp promoter and T7 promoter, etc.), ribosome binding site for initiation of translation and transcription/translation termination sequences are generally included. In addition, for example, in the case of eukaryotic cell as a host, a promoter derived from the genome of mammalian cells (example: metallothionine promoter, β-actin promoter, human hemoglobin promoter and human muscle creatine promoter) or a promotor derived from mammalian viruses (example: adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, Moloney Virus promotor, Epstein Barr Virus (EBV) promoter, and Rous sarcoma virus (RSV) promoter) can be used, and a polyadenylation sequence can be included generally as a transcription termination sequence.

In some cases, the vector may be fused with other sequences to facilitate the purification of the antibody expressed. The sequence to be fused is, for example, glutathione S-transferase (Pharmacia, USA), maltose binding protein (NEB, USA), FLAG (IBI, USA) and 6×His (hexahistidine; Qiagen, USA).

The vector contains an antibiotic resistance gene commonly used in the art as a selection marker, for example a resistant gene to ampicillin, gentamicin, carbenicillin, chloramphenicol, streptomycin, kanamycin, geneticin, neomycin and tetracycline. In another aspect, the present disclosure relates to a cell transformed with the above-mentioned expression vector. Cells used to produce the antibodies may be prokaryote, yeast and a higher eukaryotic cell, but not limited thereto.

Prokaryotic host cells such as Escherichia coli, Bacillus strains such as Bacillus subtilis and Bacillus thuringiensis, Streptomyces, Pseudomonas (e.g., Pseudomonas putida), Proteus mirabilis and Staphylococcus (for example, Staphylococcus carnosus), can be used.

Exemplary useful animal host cell lines are COS-7, BHK, CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TM, MRC 5, FS4, 3T3, MN, A549, PC12, K562, PER.C6, SP2/0, NS-0, U205, or HT1080, but not limited thereto.

The cells can be cultured in various media. Any commercial culture media can be used without limitation. All other essential supplements known to those skilled in the art may be included in an appropriate concentration. Culture conditions, such as temperature, pH, etc., and selected host cells are known to those skilled in the art.

Recovery of the antibody or antigen-binding fragment thereof can be made through removing impurities for example using centrifugation or ultrafiltration, and for example, using affinity chromatography, etc. Additional extra purification technology such as anion or cation exchange chromatography, hydrophobic interaction chromatography, hydroxyapatite chromatography, and the like can be used. In an aspect, the nucleic acid molecule encodes a polypeptide comprising a heavy chain monomer (i.e., the first polypeptide and/or the second polypeptide) and/or the light chain monomer (i.e., the third polypeptide and/or the fourth polypeptide) of the multivalent antibody binding molecule of the disclosure.

In an aspect of the disclosure is a set of one or more polynucleotides wherein each polynucleotide encodes at least one of the monomer chains of the multivalent antibody binding molecule of the disclosure, such that all chains of said multivalent antibody binding molecule are encoded. In a further aspect of the disclosure, the set of one or more polynucleotides encodes two chains of the multivalent antibody binding molecule. In a further aspect of the disclosure, the set of one or more polynucleotides encodes three chains of the multivalent antibody binding molecule. In a further aspect of the disclosure, the set of one or more polynucleotides encodes four chains of the multivalent antibody binding molecule.

In an aspect of the disclosure the nucleic acid molecules encode polypeptides of the disclosure further comprising a signal peptide. In an aspect of the disclosure the nucleic acid molecules encode polypeptides of the disclosure which do not comprise a signal peptide.

An aspect of this disclosure is a pharmaceutical composition comprising a FZD Agonist or a nucleic acid molecule, expression cassette, vector, a set of nucleic acid molecules or a set of vectors encoding a FZD Agonist described herein and a pharmaceutically acceptable carrier, diluent or excipient. The pharmaceutical composition may further comprise an additional agent, e.g., a second therapeutic antibody e.g., an anti-VEGF antibody (aflibercept, ranibizumab and bevacizumab), a growth factor, e.g., VEGF, or an agent that activates a Wnt pathway, e.g., the small molecule CHIR99021, a Norrin or R-Spondin, or a nucleic acid molecule, expression cassettes and vectors that encode the agent. The pharmaceutical composition may consist of or consist essentially of a FZD Agonist, or a nucleic acid molecule, an expression cassette or vector encoding an FZD Agonist described herein, and a pharmaceutically acceptable diluent, carrier or excipient. Suitable carriers, diluents and excipients, and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution may be e.g., from about 5 to about 8, from about 5 to 7.5 or from about 6 to 7. Further carriers include sustained release preparations such as semipermeable matrices of solid hydrophobic polymers containing the agonist, which matrices are in the form of shaped articles, e.g., films, liposomes or microparticles. It will be apparent to those persons skilled in the art that certain carriers may be more preferable depending upon, for instance, the route of administration and concentration of the FZD Agonists being administered.

This disclosure also includes methods for using the FZD Agonists, described herein. An aspect of this disclosure is a method for activating a Wnt signaling pathway in a cell, comprising contacting a cell having an FZD receptor and a LRP5 receptor, with a multivalent antibody binding molecule of this disclosure that binds the FZD, e.g., FZD4, and LRP5 in an amount effective to activate Wnt signaling. It has been reported that the Norrin-FZD4 pathway plays a role in retinal angiogenesis (see Wang et al. Cell. 2012; 151(6):1332-1344; Braunger B M, Tamm E R. Adv Exp Med Biol. 2012; 723:679-683; Ohlmann A, Tamm E R. Prog Retin Eye Res. 2012; 31(3):243-257 and; Ye et al. Trends Mol Med. 2010; 16(9):417-425). Signaling through Norrin-FZD4 pathway is necessary for development and maintenance of retinal vasculature. Mutations affecting genes of this pathway may result in several vitreoretinopathies, such as Norrie Discase, Familial Exudative Vitreoretinopathy (FEVR), and Pseudoglioma and Osteoporosis Syndrome. Additionally, Retinopathy of Prematurity (ROP) has been associated with mutations in this Norrin-FZD4 pathway, and Wnt-pathway mutations have been reported in Coats Disease and Persistent Fetal Vasculature (PFV). FZD4 signaling activated by Norrin and/or WNT7A/B pathway is also associated with central nervous system (CNS) blood brain barrier development and homeostasis. Genetic ablation of the Norrin, FZD4, LRP5, LRP6 and the co-receptor Tetraspanin-12 (Tspan-12) result in defective angiogenesis and barrier disruption in the retinal and/or cerebellar vessels (Cho et al. (2017) Neuron 95, 1056-1073; Zhou et al., (2014) J Clin Invest 124:3825-3846). Thus, a functional Wnt signaling system plays a key fundamental role in the development of a sufficient vascular and neural network in the eye and retina to support vision and in the CNS to support blood-brain barrier (BBB) development and homeostasis.

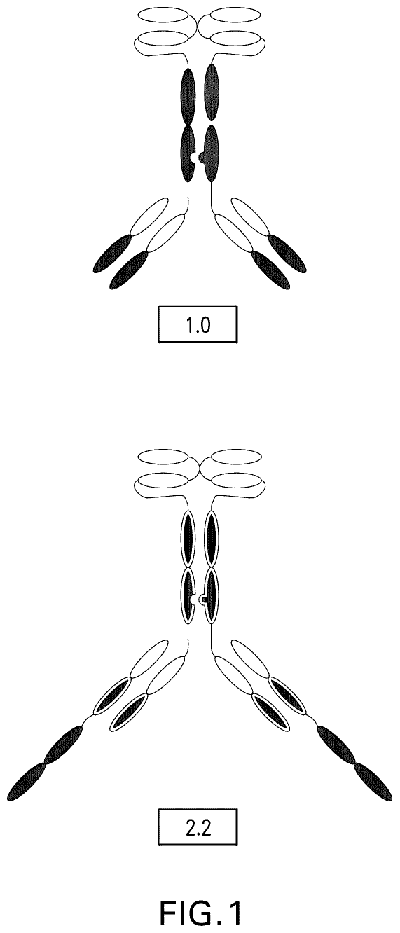

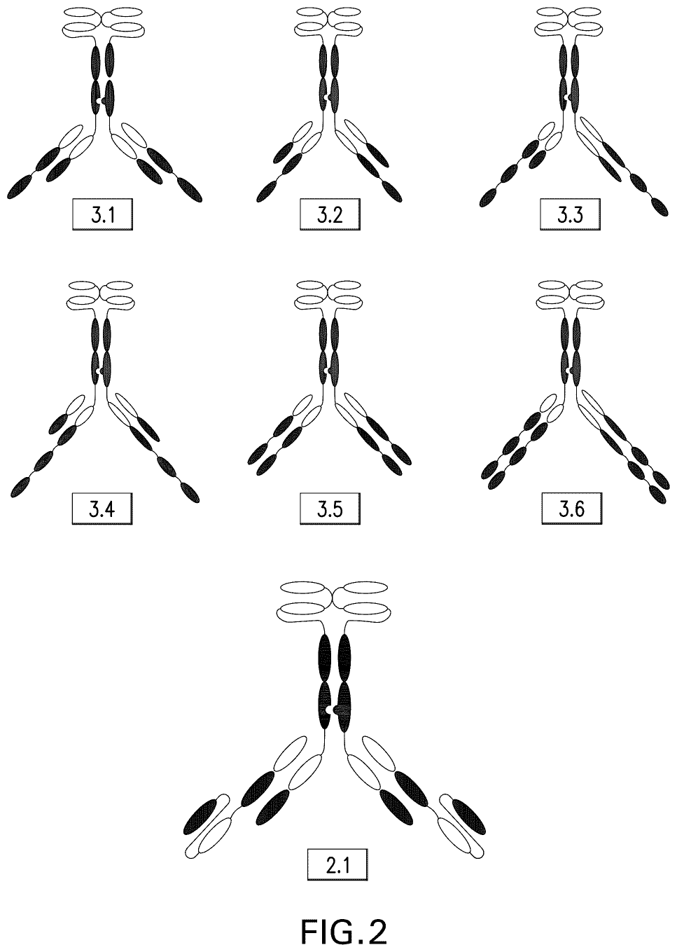

An aspect of this disclosure is a method for promoting and/or maintaining retinal vasculature by treating eye tissue, e.g., retinal tissue, with an effective amount of a pharmaceutical composition comprising a multivalent antibody molecule of this disclosure, e.g., a multivalent antibody molecule that binds FZD4 and LRP5/6, an FZD4 agonist, having the structures illustrated in FIGS. 1-2 through local or systemic administration. Also, an aspect of this disclosure is a method for promoting and/or maintaining BBB vasculature by treating a subject in need thereof with an effective amount of a pharmaceutical composition of this disclosure, e.g., a composition comprising an FZD4 agonists having the structures depicted in FIGS. 1-2. The BBB is initiated during development and its integrity remains vital for homeostasis and neural protection throughout life.

A further aspect of this disclosure is a method for treating a subject having a disorder characterized by vascular leakage, particularly retinal vascular leakage, and/or endothelial cell leakage, or disorders characterized by reduced retinal or brain endothelial cell barrier functions or a compromised BBB or BRB, e.g., diabetic retinopathy, retinopathy of prematurity, Coat's disease, familial exudative vitreoretinopathy (FEVR), Norrie disease, macular degeneration (including neovascular or wet age-related macular degeneration), diabetic macular edema, and pediatric vitreoretinopathies, by administering to such subject an effective amount of a pharmaceutical composition of the disclosure, e.g., a composition comprising an FZD4 agonist having the structures depicted in FIGS. 1-2. An effective amount of such composition is an amount sufficient, e.g., to increase or restore endothelial cell barrier functions, thereby reducing vascular leakage in such subject. The subject may be a fetus. The FZD4 agonists of this disclosure particularly the FZD4 agonist in the diabody-Fc-Fab format comprising two Fab fragments forming the FZD4 binding domain on the carboxy terminal of the Fc receptor and a binding domain for LRP5 composed of a diabody on the amino terminal of the Fc domain, e.g., as illustrated in FIGS. 1-2, activates FZD4 and β-catenin signaling in endothelial cells, promotes barrier functions and thereby reduces endothelial cell permeability and significantly enhance angiogenesis. In particular, treatment of endothelial cells, in vivo, ex vivo or in vitro, with these FZD4 agonists, preferably those with the diabody-Fc-Fab format, may enhance the development and maintenance of retinal vasculature and/or the BRB and the BBB far more effectively than other molecules that do not have this structure.

A further aspect of the disclosure is a method for treating a subject having inflammation of all or part of the intestines, also known as inflammatory bowel disease, by administering to such subject an effective amount of a pharmaceutical composition of this disclosure, e.g., a composition comprising a FZD5 Agonist. Examples of inflammatory bowel disease include, but are not limited to, Crohn's disease, and ulcerative colitis. An effective amount of such composition is an amount sufficient to reduce, ameliorate, eliminate, or treat the inflammation. A subject in need thereof includes a subject having inflammation of the mucosal of the gastrointestinal tract. The methods disclosed herein may be practiced to reduce inflammation (e.g., inflammation associated with IBD or in a tissue affected by IBD, such as gastrointestinal tract tissue, e.g., small intestine, large intestine, or colon), activate WNT signaling, or reduce any of the histological symptoms of IBD (e.g., those disclosed herein).

The FZD Agonists of the present disclosure may be administered systemically or locally, e.g., by injection (e.g., subcutaneous, intravenous, intraperitoneal, intrathecal, intraocular, intravitreal, etc.), implantation, topically, or orally. Depending on the route of administration, the FZD Agonists may be coated in a material to protect the agonists from conditions that may inactivate the agonists. The multivalent antibody binding molecules described herein may be dissolved or suspended in a pharmaceutically acceptable, preferably aqueous carrier. In addition, the composition comprising the FZD Agonists can contain excipients, such as buffers, binding agents, blasting agents, diluents, flavors, lubricants, etc. An extensive listing of excipients that can be used in such a composition, can be, for example, taken from A. Kibbe, Handbook of Pharmaceutical Excipients (Kibbe, 2000). The multivalent antibody binding molecules can also be administered together with immune stimulating substances, such as cytokines.

An aspect of this disclosure includes a method for directed differentiation of multipotent or pluripotent stem cells (PSC) or induced pluripotent stem (iPS) cells comprising culturing the cells under conditions suitable for directed differentiation wherein said culturing conditions further comprise an effective amount of a multivalent antibody binding molecule described herein. Studies in mouse and human PSCs have identified specific approaches to the addition of growth factors, including Wnt, which can induce PSC differentiation into different lineages. Methods for directed differentiation of PSCs comprising the activation of Wnt signaling are known in the art see e.g., Lam et al. (2014) Semin Nephol 34(4); 445-461; Yucer et al. (Sep. 6, 2017) Scientific Reports 7, Article number 10741. It is contemplated that the FZD Agonists, e.g., FZD4 Agonists, described herein can be used in an amount sufficient to effect activation of Wnt signaling pathways to direct differentiation of the PSCs to certain mesodermal lineages such as cardiomyocytes (cite Yoon et al. FZD4 Marks Lateral Plate Mesoderm and Signals with NORRIN to Increase Cardiomyocyte Induction from Pluripotent Stem Cell-Derived Cardiac Progenitors. Stem Cell Reports. 2018 January; 10(1):87-100. DOI: 10.1016/j.stemcr.2017.11.008.PMID: 29249665).

Also, an aspect of this disclosure is a method for making the multivalent antibody binding molecules described herein. The amino acid sequences of FZD receptors, e.g., FZD4, and the Wnt co-receptors LRP5/6, and nucleotide sequences encoding FZD receptors and the Wnt co-receptors LRP5/6, as well as antibodies and libraries of antibodies that bind FZD, e.g., FZD4, or the Wnt co-receptors LRP5/6, are readily available or can be generated using methods well known in the art (scc e.g., U.S. publication no. 2015/0232554, inventors Gurney et al. and US publication no. 2016/0194394, inventors Sidhu et al. and US20190040144, inventors Pan et al.; U.S. publication no. 2017/0166636, inventors Wu et al.; U.S. publication no. 2016/0208018, inventors Chen et al.; U.S. publication no. 2016/0053022, inventors Macheda et al.; U.S. publication no. 2015/031293, inventors Damelin et al.). And a variety of methods are known in the art for generating and screening such phage display libraries for antibodies, and antibody fragments, scFv, Fab, VL, and VH possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352:624-628 (1991); Marks et al., J. Mol. Biol. 222:581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284 (1-2): 119-132 (2004), all incorporated herein by reference. In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12:725-734 (1993). Finally, naive libraries can also be made synthetically by cloning unrearranged V-gene segments from stem cells and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360, all incorporated herein by reference. Antibodies or antibody fragments isolated from human antibody libraries are considered human antibodies or human antibody fragments herein.

### Assays

The ability of the multivalent antibody binding molecules of this disclosure to activate Wnt signaling can be confirmed by a number of assays. The multivalent antibody binding molecules of this disclosure typically initiate a reaction or activity that is similar to or the same as that initiated by the FZD receptor's natural ligand. The multivalent antibody binding molecules of this disclosure activates the Wnt signaling pathways, e.g., the canonical Wnt/β-catenin signaling pathway.

Various methods are known in the art for measuring the level of Wnt/β-catenin activation. These include but are not limited to assays that measure: Wnt/β-catenin target gene expression; LEF/TCF reporter gene expression (such as TopFLASH, superTopFLASH, pBAR); β-catenin stabilization; LRP5/6 phosphorylation; Dishevelled phosphorylation; Axin translocation from cytoplasm to cell membrane and binding to LRP5/6. The canonical Wnt/β-catenin signaling pathway ultimately leads to changes in gene expression through the transcription factors TCF1, TCF7L1, TCF7L2 and LEF1. The transcriptional response to Wnt activation has been characterized in a number of cells and tissues. As such, global transcriptional profiling by methods well known in the art can be used to assess Wnt/β-catenin signaling activation.

Changes in Wnt-responsive gene expression are generally mediated by TCF and LEF transcription factors. A TCF reporter assay assesses changes in the transcription of TCF/LEF controlled genes to determine the level of Wnt/β-catenin signaling. A TCF reporter assay was first described by Korinck, V. et al., 1997. Also known as TOP/FOP this method involves the use of three copies of the optimal TCF motif CCTTTGATC, or three copies of the mutant motif CCTTTGGCC, upstream of a minimal c-Fos promoter driving luciferase expression (pTOPFLASH and pFOPFLASH, respectively) to determine the transactivational activity of endogenous β-catenin/TCF. A higher ratio of these two reporter activities (TOP/FOP) indicates higher β-catenin/TCF activity. A newer and more sensitive version of this reporter is called pBAR and contains 12 repeats of the TCF motifs (Bicchele and Moon, Methods Mol Biol. 2008; 468:99-110, PMID: 19099249).

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

### EXAMPLES

The following examples are meant to be illustrative and should not be construed as further limiting. The contents of the figures and all references, patents, and published patent applications cited throughout this application are expressly incorporated herein by reference.

### Example 1: Expression of Constructs, TOPFlash Assay Analysis of β-Catenin Mediated Gene Reporter Activity and VEGF Neutralization Assay

The molecular formats described below in Table 2 and as described in Table 8 and FIGS. 1-2 were expressed and purified.

TABLE 2

| Molecule | Titer | Conc | Purity |
|---|---|---|---|
| Format | (mg/mL) | (mg/mL) | (%)SEC-HPLC |
| 3.1 | 0.75 | 4.67 | 98.8 |
| 3.2 | 1.35 | 4.34 | 99.0 |
| 3.3 | 0.69 | 4.5 | 98.6 |
| 3.4 | 0.87 | 4.95 | 98.9 |
| 3.5 | 0.51 | 6.42 | 97.9 |
| 3.6 | 1.01 | 0.60 | 99.6 |
| 2.2 | 0.50 | 4.58 | 98.1 |

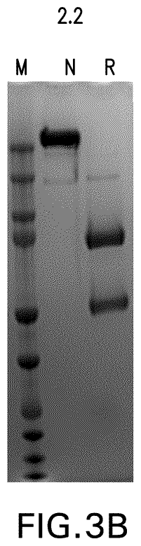

Molecules 3.1-3.6 and 2.2 were tested in a TOPFlash assay to monitor β-catenin mediated gene reporter activity. Chinese hamster ovary (CHO) cells were transduced with two lentiviral constructs. Details of the transcription units incorporated into the lentivirus are shown in FIG. 4. Such constructs include the highly and constitutively active MND promoter (myeloproliferative sarcoma virus enhancer, negative control region deleted to resist transcriptional silencing, d1587rev primer-binding site substituted), which is used to express both the LRP5 (NM_002335.4) and FZD4 genes (NM_012193.4). Human Mesoderm Development LRP chaperone (MESD) gene (NM_015154.3) and LRP5 are expressed as a single transcription unit which contains the T2A self-cleaving peptide allowing production of the two separate proteins LRP5 and MESD. MESD, is included in the construct design to assist with the folding and plasma membrane trafficking of LRP5.

The two lentiviral constructs were used to transduce CHO cells at a multiplicity of infection of 0.6 and 2.0 respectively and stable pools were selected using blasticidin and puromycin antibiotic selection. Clonal cell lines were subsequently isolated by limiting dilution and used to assess the β-catenin stabilization induced by the molecules.

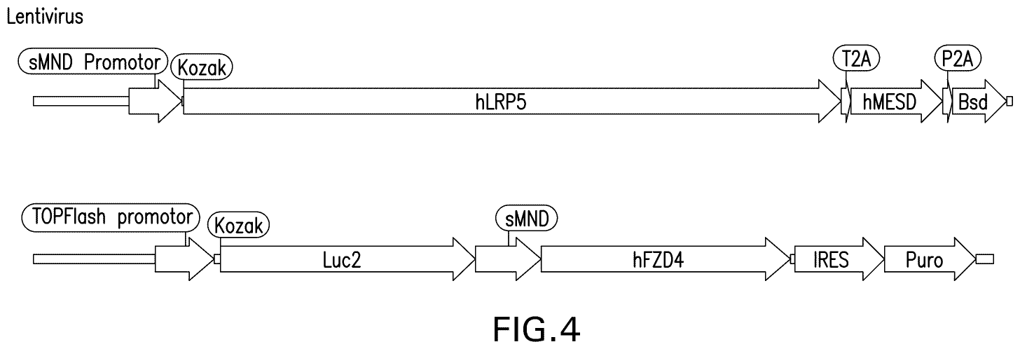

The TOPFlash assay was performed by plating 20,000 cells into a 96 well plate and test molecules, which had been previously 3-fold serially diluted from 20 μg/mL, are added and the plate is incubated at 37° C., in a 5% CO2 incubator for 24 hours. Following incubation, 75 μL of Steady-Glo® reagent was added to each well and incubated at room temperature in the dark for 35 mins. Luminescence output was measured using a SpectraMax® i3x Plate Reader, data is plotted in FIG. 5.

A commercially available VEGF reporter BIOASSAY kit (Promega®, Catalogue No. GA2005) was used to measure VEGF signaling inhibition for a control molecule. The VEGF Bioassay is a bioluminescent cell-based assay that can measure VEGF activation and inhibition of the VEGFR-2 receptor (KDR) using bioluminescence as a readout. HEK293 (human embryonic kidney) cells were engineered to express exogenous VEGFR-2. Upon activation, the receptor transduces intracellular signals which result in the activation of the modified luciferase reporter gene luc2P, due to the presence of VEGFR response element upstream of the luc2p promoter. The luciferase expression produces a bioluminescent signal, which can be detected by using Bio-Glo™ Luciferase Assay System and a standard luminometer.

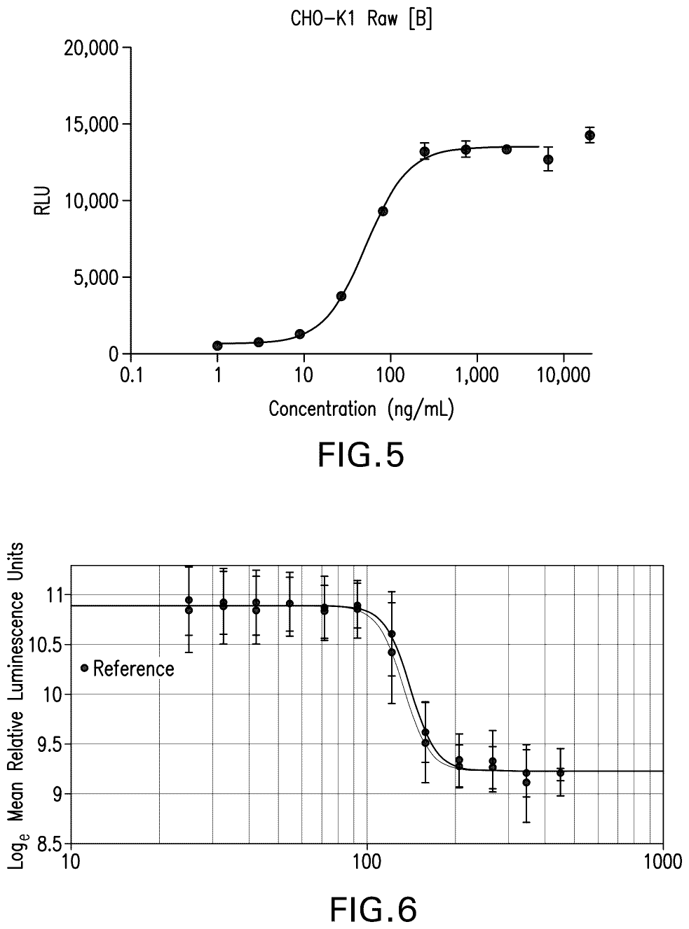

KDR/NFAT-RE HEK293 cells were thawed and plated (at ˜4×10⁴ cell/well) in DMEM, high glucose, GlutaMAX™ Growth (10%) media for immediate use. Test and reference samples were serially prediluted 1.3-fold starting at 1350 ng/mL in DMEM high glucose GlutaMAX™ Growth (10%) media. 40 μL VEGF at a final concentration of 100 ng/ml and 40 μL of test sample or positive control were added to the assay plates and incubated at 37° C., in a 5% CO2 incubator for 6 hours. Following incubation 75 μL of BioGlo reagent was added to each well and incubated at room temperature in the dark for 5-10 mins. Luminescence output was measured using a SpectraMax® i3x Plate Reader and data plotted in FIG. 6. Data are present in Table 3 for molecules 3.1-3.6 (See FIG. 2 and Table 8), and a VEGF-Trap control molecule comprising the same VEGF binding domain as contained in molecule 2.2 as described in FIG. 1 and Table 8 (i.e., SEQ ID NO: 49).

TABLE 3

| | VEGF Neutralization | FZD4 activation |
|---|---|---|
| Molecule | IC50 ng/ml (nM) | EC50 ng/nl (nM) |
| VEGF-Trap | 95.2 (0.475 nM) | N/A |
| control | | |
| 1.0 | N/A | 16.34 (0.08 nM) |
| 3.1 | 114.4 (0.51 nM) | 19.27 (0.09 nM) |
| 3.2 | 116.9 (0.52 nM) | 18.35 (0.08 nM) |
| 3.3 | 126.0 (0.5 nM) | 17.04 (0.07 nM) |
| 3.4 | 129.9 (0.52 nM) | 19.19 (0.08 nM) |
| 3.5 | 58.5 (0.23 nM) | 25.30 (0.10 nM) |
| 3.6 | 78.5 (0.26 nM) | 32.74 (0.11 nM) |

### Example 2: Wnt Agonism Assay Method

CHO-K1 TOPFlash B2 MCB cells co-expressing human FZD4 and LRP5 were used as a reporter cell line to assess functional Wnt pathway activation upon exposure to test articles. In brief, cells were plated in F-12 media plus 10% fetal bovine serum at a density of 10,000 cells/well of a 96-well plate the afternoon prior to test article exposure. A compound plate of test articles was prepared by serial dilution from 50 nM top concentration in a 3-fold, 10-point dilution series in growth media. To determine Eₘₐₓ and Eₘᵢₙ (maximal and minimal response), replicate (n=8) wells of cells were treated with either 50 nM of a reference compound or vehicle-containing media only. Cells underwent a complete media exchange into test articles and were allowed to incubate for 24 hours.

The Promega ONE-Glo™ kit was used to determine extent of luciferase expression after exposure to test articles. Cell culture media was exchanged with PBS prior to a 1:1 addition of complete ONE-Glo reagent and incubated for 30 minutes at approximately 25° C. with orbital shaking at 600 rpm. A SpectraMax M5 plate reader was used to measure luminescence.

Potency determination was determined by normalizing the raw data to the Eₘₐₓ and Eₘᵢₙ signals, then a 4PY fit was performed in GraphPad Prism®. EC50 values are represented as a geomean of multiple assays. Average values for the normalized maximal and minimal effect of each test article are also reported.

TABLE 4

| Emax/Emin | | | |
|---|---|---|---|
| Average | EC50 | Eₘₐₓ | Eₘᵢₙ |
| DAB032364 | 0.13 | 110.4 | 0.9 |
| DAB032370 | 0.10 | 107.7 | 0.35 |
| DAB032366 | 0.12 | 95.8 | 0.3 |
| DAB030448 | 0.10 | 103.3 | 0.0 |
| DAB030452 | 0.15 | 99.5 | 1.2 |
| DAB032574 | 0.15 | 91.5 | −3.5 |
| DAB032573 | 0.12 | 102.0 | 3.1 |

### Example 4: WNT Biacore Method

Multispecific molecules were tested for binding affinity to human VEGF121 and VEGF165, rabbit VEGF121 and VEGF165, human Frizzled 4 and LRP5 using a Biacore 8K biosensor. Affinity for human Frizzled 4 and LRP5 was tested both for the unbound molecules as well as the molecules bound to human VEGF165. Antibody from a Human Antibody Capture Kit, Type 2 (Cytiva) was amine coupled at a saturating level to a Series S CM5 sensor chip (Cytiva). Multispecific molecule ligands were captured at 50 nM for 15 seconds. Human VEGF 165 was injected at 20 nM for 40 seconds. Analyte was then injected for 180 seconds and dissociation was monitored for 570 to 1200 seconds. Analyte concentrations were 0.08 nM to 20 nM for all VEGF species, 16 nM to 400 nM for human Frizzled 4, and 6 nM to 150 nM for LRP5. All dilutions were done in the running buffer: 0.1 M HEPES, 1.5 M NaCl, 0.03 M EDTA and 0.5% v/v surfactant polysorbante 20 (PS20). The capture surface was regenerated with a 30 second injection of 3 M MgCl₂ between binding cycles. The data were double referenced and fit to a 1:1 binding model using Biacore Insight software. Data are presented in Table 5.

TABLE 5
Binding Affinity

| | | | | | | Ligand | Surface |
|---|---|---|---|---|---|---|---|
| | | k_{ON} | k_{OFF} | | Rmax | Level | Activity |
| Analyte | Ligand | (1/Ms) | (1/s) | K_{D} (M) | (RU) | (RU) | (%) |
| 1 | DAB030448 | 2.1E+07 | 2.6E−04 | 1.3E−11 | 67 | 331 | 113 |
| | DAB030452 | 5.9E+07 | 3.1E−04 | 5.2E−12 | 88 | 281 | 104 |
| | DAB032366 | 2.2E+07 | 1.4E−04 | 6.5E−12 | 56 | 283 | 109 |
| | DAB032370 | 1.9E+07 | 2.1E−04 | 1.1E−11 | 63 | 311 | 113 |
| | DAB032364 | 3.0E+07 | 1.7E−04 | 5.8E−12 | 92 | 287 | 106 |
| 2 | DAB030448 | 1.1E+07 | 2.3E−04 | 2.1E−11 | 44 | 332 | 94 |
| | DAB030452 | 9.5E+06 | 1.7E−04 | 1.8E−11 | 58 | 281 | 88 |
| | DAB032366 | 1.1E+07 | 1.9E−04 | 1.6E−11 | 36 | 283 | 92 |
| | DAB032370 | 1.1E+07 | 2.0E−04 | 1.9E−11 | 41 | 310 | 95 |
| | DAB032364 | 1.2E+07 | 1.6E−04 | 1.4E−11 | 60 | 287 | 90 |
| 3 | DAB030448 | 1.8E+07 | 2.5E−04 | 1.3E−11 | 63 | 326 | 107 |
| | DAB030452 | 2.8E+07 | 2.0E−04 | 7.3E−12 | 84 | 274 | 101 |
| | DAB032366 | 3.7E+07 | 2.1E−04 | 5.8E−12 | 54 | 282 | 106 |
| | DAB032370 | 2.0E+07 | 2.3E−04 | 1.1E−11 | 59 | 304 | 108 |
| | DAB032364 | 3.0E+07 | 1.8E−04 | 6.1E−12 | 89 | 284 | 103 |
| 4 | DAB030448 | 1.2E+07 | 2.5E−04 | 2.1E−11 | 43 | 326 | 93 |
| | DAB030452 | 8.6E+06 | 1.8E−04 | 2.2E−11 | 57 | 273 | 89 |
| | DAB032366 | 1.2E+07 | 1.9E−04 | 1.5E−11 | 36 | 282 | 92 |
| | DAB032370 | 1.1E+07 | 2.1E−04 | 2.0E−11 | 40 | 303 | 94 |
| | DAB032364 | 9.1E+06 | 1.7E−04 | 1.9E−11 | 61 | 283 | 91 |
| 5 | DAB030448 | 5.3E+04 | 1.0E−03 | 2.0E−08 | 39 | 309 | 89 |
| | DAB030452 | 5.1E+04 | 1.0E−03 | 2.0E−08 | 28 | 263 | 89 |
| | DAB032366 | 5.9E+04 | 1.1E−03 | 1.8E−08 | 35 | 274 | 90 |
| | DAB032370 | 5.4E+04 | 9.8E−04 | 1.8E−08 | 37 | 294 | 90 |
| | DAB032364 | 5.4E+04 | 1.0E−03 | 1.9E−08 | 31 | 280 | 93 |
| 6 | DAB030448 + | 5.1E+04 | 9.3E−04 | 1.8E−08 | 38 | 309 | 87 |
| | Hu VEGF165 | | | | | | |
| | DAB030452 + | 5.2E+04 | 9.4E−04 | 1.8E−08 | 27 | 263 | 88 |
| | Hu VEGF165 | | | | | | |
| | DAB032366 + | 6.1E+04 | 1.0E−03 | 1.6E−08 | 35 | 274 | 90 |
| | Hu VEGF165 | | | | | | |
| | DAB032370 + | 5.0E+04 | 9.0E−04 | 1.8E−08 | 37 | 293 | 89 |
| | Hu VEGF165 | | | | | | |
| | DAB032364 + | 4.9E+04 | 9.1E−04 | 1.8E−08 | 30 | 280 | 91 |
| | Hu VEGF165 | | | | | | |
| 7 | DAB030448 | 6.5E+04 | 1.1E−03 | 1.6E−08 | 71 | 309 | 37 |
| | DAB030452 | 6.3E+04 | 1.2E−03 | 1.9E−08 | 56 | 264 | 41 |
| | DAB032366 | 4.9E+04 | 1.1E−03 | 2.3E−08 | 56 | 274 | 34 |
| | DAB032370 | 6.5E+04 | 1.0E−03 | 1.6E−08 | 72 | 294 | 40 |
| | DAB032364 | 6.6E+04 | 1.1E−03 | 1.7E−08 | 63 | 280 | 44 |
| 8 | DAB030448 + | 6.0E+04 | 1.0E−03 | 1.7E−08 | 69 | 309 | 37 |
| | Hu VEGF165 | | | | | | |
| | DAB030452 + | 5.9E+04 | 1.1E−03 | 1.9E−08 | 60 | 263 | 44 |
| | Hu VEGF165 | | | | | | |
| | DAB032366 + | 2.2E+04 | 1.4E−03 | 6.3E−08 | 32 | 274 | 19 |
| | Hu VEGF165 | | | | | | |
| | DAB032370 + | 6.2E+04 | 1.0E−03 | 1.6E−08 | 70 | 293 | 39 |
| | Hu VEGF165 | | | | | | |
| | DAB032364 + | 6.1E+04 | 1.1E−03 | 1.8E−08 | 66 | 279 | 46 |
| | Hu VEGF165 | | | | | | |

### Example 3: Axin2 qPCR Assay

bEnd.3 cells (#CRL-2299 ATCC) were maintained in Dulbecco's modified Eagle's medium (DMEM) supplemented with 10% Fetal bovine serum at 37° C. in a 5% CO2 environment. Cells were passaged using trypsin 0.25%-EDTA solution at a split ratio of 1:3-5 and cells were used between p2 and p6 for the experiments. For the treatment, cells were seeded at a density of 30,000 per well in 96-well plates 24 h prior to treatment. After 6 h treatment, cells were lysed with lysis binding mix from MagMAX mirVana Total RNA isolation kit (Thermo Fisher Scientific Inc., Foster City, CA) using vendor suggested procedures and proceeded to RNA extraction.

For real-time PCR analysis, total RNA was isolated from cellular samples using the MagMAX mirVana Total RNA isolation kit (Thermo Fisher Scientific Inc., Foster City, CA) according to the manufacturer's instructions. Total RNA samples were qualified and quantified on the Fragment Analyzer (Agilent, Santa Clara, CA) per manufacturer's instructions.

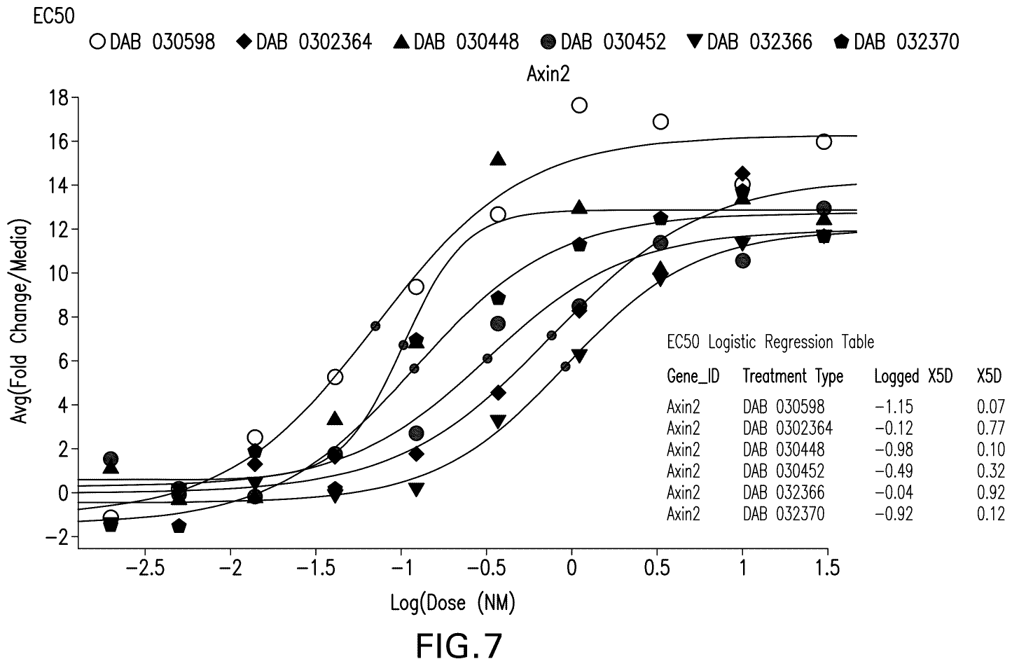

DNase-treated total RNA was reverse-transcribed using QuantiTect Reverse Transcription (Qiagen, Valencia, CA) according to manufacturer's instructions. 20× primer assays were obtained commercially from Thermo Fisher Scientific (Foster City, CA). Gene specific pre-amplification was done on 2-5 ng cDNA per Standard BioTools Biomark manufacturer's instructions (Standard BioTools, Foster City). Real-time quantitative PCR was performed on the Standard BioTools Biomark HD using 20× Taqman primer assays (Thermo Fisher Scientific Inc, Foster City, CA) with Taqman Fast Universal PCR Master Mix with no AmpErase UNG. Samples and primers were run on a 192.24 Dynamic Array per manufacturer's instructions (Standard BioTools, Foster City). Ubiquitin B levels were measured in a separate reaction and used to normalize the data by the Δ Ct method. Using the mean cycle threshold value for ubiquitin and the gene of interest for each sample, the equation: 1.8\hat{ }(Ct UbiquitinB minus Ct gene of interest)×10⁴ was used to obtain the normalized values. Results are shown in Table 6 and FIG. 7.

TABLE 6

| | Compound | | | | |
|---|---|---|---|---|---|
| | HC CT | | LC / HC CT | | HC NT |
| Name | DAB032370 | DAB030448 | DAB032364 | DAB030452 | DAB032366 |
| Potency (EC50 | 0.14 | 0.14 | 0.50 | 0.37 | 0.46 |
| WNT Activation- | | | | | |
| Axin2 mRNA) | | | | | |
| Geomean (nM) | | | | | |

Axin2 is a key component of canonical Wnt pathway signaling and Axin2 expression evidences Wnt activation. As shown in Table 6, the potency of Wnt activation as measured by Axin2 expression was determined, and DAB032370 and DAB030448 had the highest Wnt potency as shown by their respective 0.14 nM EC50 potency.

### Example 4: In Vitro Functional Assay: Rescue of VEGF-Induced Hyper-Permeability in Mouse bEnd.3 EC TEER

bEnd.3 cells (#CRL-2299 ATCC) were maintained in Dulbecco's modified Eagle's medium (DMEM) supplemented with 10% Fetal bovine serum at 37° C. in a 5% CO2 environment. Cells were passaged using trypsin 0.25%-EDTA solution at a split ratio of 1:3-5 and cells were used between p2 and p6 for the experiments. 96-well ECIS Trans-Epithelial/endothelial Electrical Resistance (TEER) plate (Applied Biophysics) was first coated with 50 ug/ml human fibronectin (ThermoFisher) for 1 hr at room temperature, followed by washing twice with PBS, and treated with 10 mM L-Cysteine for 30 min at room temperature. The plate was then washed twice with PBS, and replenished with 100 ul culture medium and placed onto the ECIS instrument for setup.

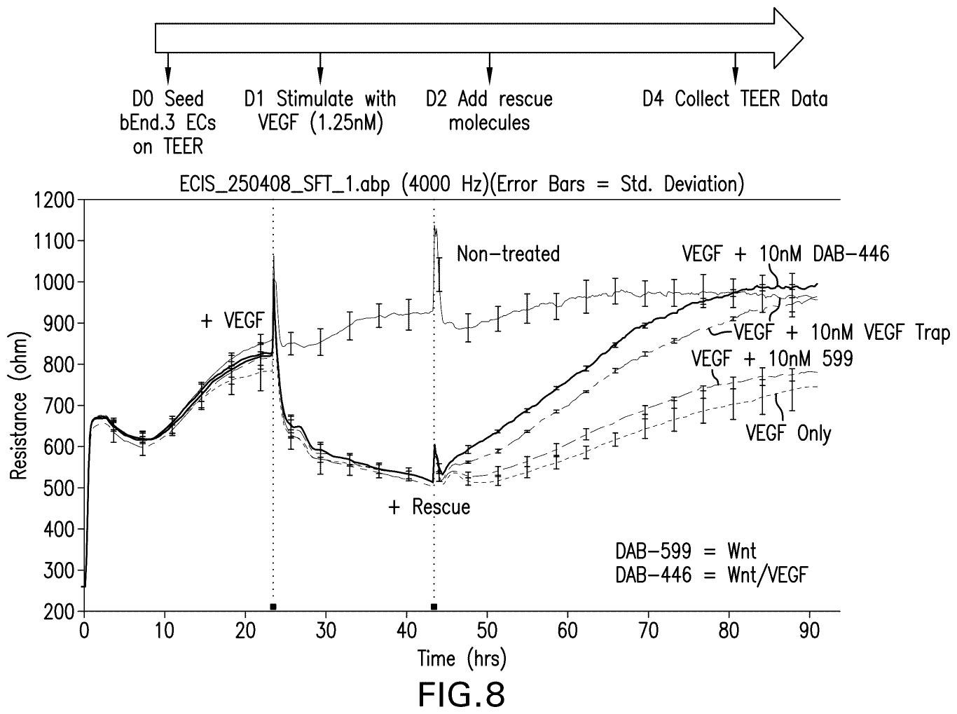

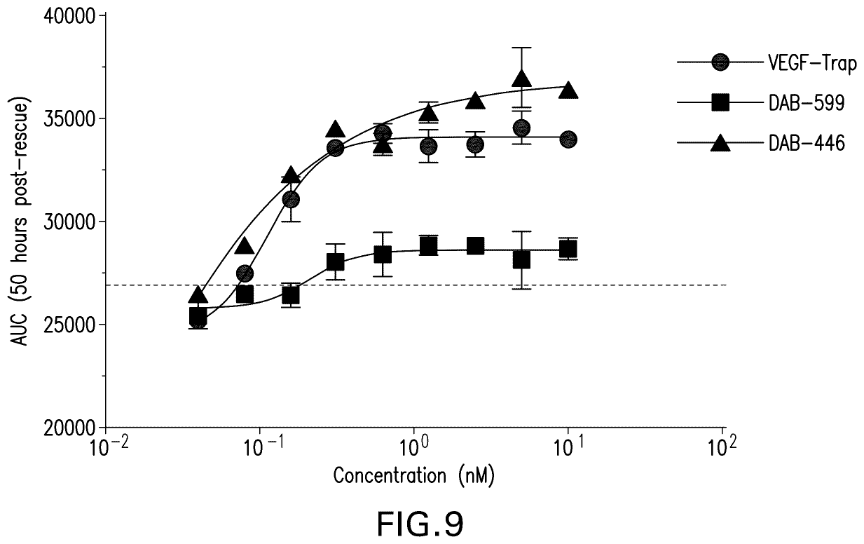

Dissociated bEnd.3 single cell suspension was plated on a TEER plate at the density of 36 k cells per well in 150 ul medium and loaded onto ECIS for continuous TEER measurement. 24 h post seeding, cells were treated with 50 ng/ml VEGF-A165 (Peprotech). 48 h post seeding, various concentrations of rescue compounds were added. 96 h post seeding, the TEER measurement was stopped, and data was exported for analysis as shown in Table 7 and FIGS. 8-9.

TABLE 7

| | | Eₘₐₓ (% Increased |
|---|---|---|
| Molecule | EC50 (nM) | over VEGF-A165) |
| VEGF Trap | 0.11 | 26% |
| (Anti-VEGF) | | |
| DAB-599 | 0.21 | 8% |
| (Wnt Only) | | |
| DAB-446 | — | 35% |
| (Wnt/VEGF | | |

These data in Example 4 evidence rescue of VEGF-induced hyper-permeability in the mouse bEnd.3 cells. Wnt signaling combined with anti-VEGF had a greater effect on Eₘₐₓ (Table 7) and showed the greatest rescue in permeability (FIG. 8) as compared to anti-VEGF alone and Wnt signaling alone, demonstrating that the combination of the effects of Wnt agonism and anti-VEGF activity showed improvement over the individual agents.

The disclosed subject matter is not to be limited in scope by the specific aspects and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other aspects are within the following claims.

TABLE 8
Sequences

| SEQ | | Sequence |
|---|---|---|
| ID | | |
| NO: | Description | |
| 1 | LRP5-1 | FSSSSI |
| | HCDR1 | |
| 2 | LRP5-1 | SISSSYGYTY |
| | HCDR2 | |
| 3 | LRP5-1 | SWAM |
| | HCDR3 | |
| 4 | LRP5-1 | SVSSA |
| | LCDR1 | |
| 5 | LRP5-1 | SASSLYS |
| | LCDR2 | |
| 6 | LRP5-1 | YWAYYSPI |
| | LCDR3 | |
| 7 | LRP5-2 | DFTAYAM |
| | HCDR1 | |
| 8 | LRP5-2 | SIYPSGGYTA |
| | HCDR2 | |
| 9 | LRP5-2 | RSYYFAL |
| | HCDR3 | |
| 10 | LRP5-2 | SVSSA |
| | LCDR1 | |
| 11 | LRP5-2 | SASDLYS |
| | LCDR2 | |
| 12 | LRP5-2 | YAGAGLI |
| | LCDR3 | |
| 13 | LRP5-1 VH | EVQLVESGGGLVQPGGSLRLSCAASGFDESSSSIHWVRQA |
| | | PGKGLEWVASISSSYGYTYYADSVKGRFTISADTSKNTAY |
| | | LQMNSLRAEDTAVYYCARSWAMDYWGQGTLVTVSS |
| 14 | LRP5-1 VL | DIQMTQSPSSLSASVGDRVTITCRASQSVSSAVAWYQQKP |
| | | GKAPKLLIYSASSLYSGVPSRESGSRSGTDFTLTISSLQP |
| | | EDFATYYCQQYWAYYSPITFGQGTKVEIK |
| 15 | LRP5-2 VH | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSS |
| 16 | LRP5-2 VL | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIK |
| 17 | WT CH2 | LLGGPSVFLFPPKPKDTLMISRTPEVTCVVVDVSHEDPEV |
| | | KFNWYVDGVEVHNAKTKPREEQYNSTYRVVSVLTVLHQDW |
| | | LNGKEYKCKVSNKALPAPIEKTISKAK |
| 18 | CH2 LALA | AAGGPSVFLFPPKPKDTLMISRTPEVTCVVVDVSHEDPEV |
| | | KFNWYVDGVEVHNAKTKPREEQYNSTYRVVSVLTVLHQDW |
| | | LNGKEYKCKVSNKALPAPIEKTISKAK |
| 19 | CH2 LALAPS | AAGGPSVFLFPPKPKDTLMISRTPEVTCVVVDVSHEDPEV |
| | | KFNWYVDGVEVHNAKTKPREEQYNSTYRVVSVLTVLHQDW |
| | | LNGKEYKCKVSNKALPASIEKTISKAK |
| 20 | WT CH3 | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| 21 | CH3 Knob-1 | GQPREPQVYTLPPSREEMTKNQVSLWCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| 22 | CH3 Hole-1 | GQPREPQVYTLPPSREEMTKNQVSLSCAVKGFYPSDIAVE |
| | | WESNGQPENNYKTTPPVLDSDGSFFLVSKLTVDKSRWQQG |
| | | NVFSCSVMHEALHNHYTQKSLSLSPGK |
| 23 | CH3 Knob-2 | GQPREPMVFDLPPSREEMTKNQVSLWCMVKGFYPSDIAVE |
| | | WESNGQPENNYKTTPPVLDSDGSFFLYSKLTVDKSRWQQG |
| | | NVFSCSVMHEALHNHYTQKSLSLSPGK |
| 24 | CH3 Hole-2 | GQPREPQVYTLPPIRELMTSNQVSLSCAVKGFYPSDIAVE |
| | | WESNGQPENNYKTTPPVLDSDGSFFLVSKLTVDKSRWQQG |
| | | NVFSCSVMHEALHNHYTQKSLSLSPGK |
| 25 | Db1 | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIK |
| 26 | Db2 | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIK |
| 27 | IgG1 Upper | DKTHT |
| | Hinge Domain | |
| 28 | IgG1 Hinge | DKTHTCPPCPAPE |
| | Domain | |
| 29 | G4S linker | GGGGS |
| | Subunit | |
| 30 | Linker | GGGGSGGGS |
| 31 | Linker | GGGGSGGGGSEPKSC |
| 32 | Linker | GGGGSGGGGSEPKSS |
| 33 | Linker | GGGGSGGGGSEPKSSDKTHT |
| 34 | Linker | GGGSGGGSGGGSGGGSGSTG |
| 35 | FZD4 HCDR1 | LSSYSM |
| 36 | FZD4 HCDR2 | YISSYDSITD |
| 37 | FZD4 HCDR3 | PAVGHMAF |
| 38 | FZD4 LCDR1 | SVSSA |
| 39 | FZD4 LCDR2 | SASSLYS |
| 40 | FZD4 LCDR3 | WYNAPI |
| 41 | FZD4 VH | EVQLVESGGGLVQPGGSLRLSCAASGFTLSSYSMHWVRQA |
| | | PGKGLEWVAYISSYDSITDYADSVKGRFTISADTSKNTAY |
| | | LQMNSLRAEDTAVYYCARPAVGHMAFDYWGQGTLVTVSS |
| 42 | FZD4 VL | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIK |
| 43 | Cₖ | RTVAAPSVFIFPPSDEQLKSGTASVVCLLNNFYPREAKVQ |
| | | WKVDNALQSGNSQESVTEQDSKDSTYSLSSTLTLSKADYE |
| | | KHKVYACEVTHQGLSSPVTKSFNRGEC |
| 44 | FZD4 Light | DIQMTQSPSSLSASVGDRVTITCRASQSVSSAVAWYQQKP |
| | Chain | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 45 | WT CH1 | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSC |
| 46 | CH1-Upper | ASTKGPSVFPLAPSSKSTSGGTAALGCLVKDYFPEPVTVS |
| | Hinge | WNSGALTSGVHTFPAVLQSSGLYSLSSVVTVPSSSLGTQT |
| | | YICNVNHKPSNTKVDKKVEPKSCDKTHT |
| 47 | VEGFR1 | SDTGRPFVEMYSEIPEIIHMTEGRELVIPCRVTSPNITV |
| | | TLKKFPLDTLIPDGKRIIWDSRKGFIISNATYKEIGLLTC |
| | | EATVNGHLYKTNYLTHRQTNTII |
| 48 | VEGFR2 | DVVLSPSHGIELSVGEKLVLNCTARTELNVGIDENWEYPS |
| | | SKHQHKKLVNRDLKTQSGSEMKKFLSTLTIDGVTRSDQGL |
| | | YTCAASSGLMTKKNSTFVRVHEK |
| 49 | VEGFR1 + VEG | SDTGRPFVEMYSEIPEIIHMTEGRELVIPCRVTSPNITVT |
| | FR2 | LKKFPLDTLIPDGKRIIWDSRKGFIISNATYKEIGLLTCE |
| | | ATVNGHLYKTNYLTHRQTNTIIDVVLSPSHGIELSVGEKL |
| | | VLNCTARTELNVGIDENWEYPSSKHQHKKLVNRDLKTQSG |
| | | SEMKKFLSTLTIDGVTRSDQGLYTCAASSGLMTKKNSTFV |
| | | RVHEK |
| 50 | VHH1 HCDR1 | SYSMG |
| 51 | VHH1 HCDR2 | AISKGGYKYDSVSLEG |
| 52 | VHH1 HCDR3 | SRAYGSSRLRLADTYEY |
| 53 | VHH1 | EVQLVESGGGLVQTGDSLRLSCEVSGRTFSSYSMGWFRQA |
| | | QGKEREFVVAISKGGYKYDSVSLEGRFTISKDNAKNTVYL |
| | | QINSLKPEDTAVYYCASSRAYGSSRLRLADTYEYWGQGTQ |
| | | VTVSS |
| 54 | VHH2 HCDR1 | SMA |
| 55 | VHH2 HCDR2 | RISSGGTTAYVDSVKG |
| 56 | VHH2 HCDR3 | FSSRPNP |
| 57 | VHH2 | EVQLVESGGGLVQPGGSLRLSCVASGIRFMSMAWYRQAPG |
| | | KHRELVARISSGGTTAYVDSVKGRFTISRDNSKNTVYLQM |
| | | NSLKAEDTAVYYCNTFSSRPNPWGAGTQVTVSS |
| 58 | Signal Peptide 1 | MNLLLILTFVAAAVA |
| 59 | 1.0 Hole HC | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGSGGGSGG |
| | | GSGGGSGSTGEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHT |
| 60 | 1.0 Knob HC | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPASIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGSG |
| | | GGSGGGSGGGSGSTGEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HT |
| 61 | 1.0 LC | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ES\VTEQDSKDSTYSLSSTLTLSKADYEKHKVYACEVTHQ |
| | | GLSSPVTKSFNRGEC |
| 62 | 3.1 Hole HC | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHT |
| 63 | 3.1 Knob HC | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPASIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HT |
| 64 | 3.1 LC | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGSEVQLVESGGGLVQTGDS |
| | | LRLSCEVSGRTFSSYSMGWFRQAQGKEREFVVAISKGGYK |
| | | YDSVSLEGRFTISKDNAKNTVYLQINSLKPEDTAVYYCAS |
| | | SRAYGSSRLRLADTYEYWGQGTQVTVSS |
| 65 | 3.2 Hole HC | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GSGGGSEVQLVESGGGLVQTGDSLRLSCEVSGRTFSSYSM |
| | | GWFRQAQGKEREFVVAISKGGYKYDSVSLEGRFTISKDNA |
| | | KNTVYLQINSLKPEDTAVYYCASSRAYGSSRLRLADTYEY |
| | | WGQGTQVTVSS |
| 66 | 3.2 Knob HC | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPASIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGSGGGSEVQLVESGGGLVQTGDSLRLSCEVSGRTF |
| | | SSYSMGWFRQAQGKEREFVVAISKGGYKYDSVSLEGRFTI |
| | | SKDNAKNTVYLQINSLKPEDTAVYYCASSRAYGSSRLRLA |
| | | DTYEYWGQGTQVTVSS |
| 67 | 3.2 LC | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 68 | 3.3 Hole HC | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHT |
| 69 | 3.3 Knob HC | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPASIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HT |
| 70 | 3.3 LC | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGSEVQLVESGGGLVQTGDS |
| | | LRLSCEVSGRTFSSYSMGWFRQAQGKEREFVVAISKGGYK |
| | | YDSVSLEGRFTISKDNAKNTVYLQINSLKPEDTAVYYCAS |
| | | SRAYGSSRLRLADTYEYWGQGTQVTVSSGGGGSGGGSEVQ |
| | | LVESGGGLVQPGGSLRLSCVASGIRFMSMAWYRQAPGKHR |
| | | ELVARISSGGTTAYVDSVKGRFTISRDNSKNTVYLQMNSL |
| | | KAEDTAVYYCNTFSSRPNPWGAGTQVTVSS |
| 71 | 3.4 Hole HC | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GSGGGSEVQLVESGGGLVQTGDSLRLSCEVSGRTFSSYSM |
| | | GWFRQAQGKEREFVVAISKGGYKYDSVSLEGRFTISKDNA |
| | | KNTVYLQINSLKPEDTAVYYCASSRAYGSSRLRLADTYEY |
| | | WGQGTQVTVSSGGGGSGGGSEVQLVESGGGLVQPGGSLRL |
| | | SCVASGIRFMSMAWYRQAPGKHRELVARISSGGTTAYVDS |
| | | VKGRFTISRDNSKNTVYLQMNSLKAEDTAVYYCNTFSSRP |
| | | NPWGAGTQVTVSS |
| 72 | 3.4 Knob HC | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPASIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGSGGGSEVQLVESGGGLVQTGDSLRLSCEVSGRTF |
| | | SSYSMGWFRQAQGKEREFVVAISKGGYKYDSVSLEGRFTI |
| | | SKDNAKNTVYLQINSLKPEDTAVYYCASSRAYGSSRLRLA |
| | | DTYEYWGQGTQVTVSSGGGGSGGGSEVQLVESGGGLVQPG |
| | | GSLRLSCVASGIRFMSMAWYRQAPGKHRELVARISSGGTT |
| | | AYVDSVKGRFTISRDNSKNTVYLQMNSLKAEDTAVYYCNT |
| | | FSSRPNPWGAGTQVTVSS |
| 73 | 3.4 LC | DIQMTQSPSSLSASVGDRVTITCRASQSVSSAVAWYQQKP |
| | | GKAPKLLIYSASSLYSGVPSRESGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 74 | 3.5 Hole HC | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GSGGGSEVQLVESGGGLVQTGDSLRLSCEVSGRTFSSYSM |
| | | GWFRQAQGKEREFVVAISKGGYKYDSVSLEGRFTISKDNA |
| | | KNTVYLQINSLKPEDTAVYYCASSRAYGSSRLRLADTYEY |
| | | WGQGTQVTVSS |
| 75 | 3.5 Knob HC | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPASIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGSGGGSEVQLVESGGGLVQTGDSLRLSCEVSGRTF |
| | | SSYSMGWFRQAQGKEREFVVAISKGGYKYDSVSLEGRFTI |
| | | SKDNAKNTVYLQINSLKPEDTAVYYCASSRAYGSSRLRLA |
| | | DTYEYWGQGTQVTVSS |
| 76 | 3.5 LC | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGSEVQLVESGGGLVQTGDS |
| | | LRLSCEVSGRTFSSYSMGWFRQAQGKEREFVVAISKGGYK |
| | | YDSVSLEGRFTISKDNAKNTVYLQINSLKPEDTAVYYCAS |
| | | SRAYGSSRLRLADTYEYWGQGTQVTVSS |
| 77 | 3.6 Hole HC | EVQLVESGGGLVQPGGSLRLSCAASGFDFSSSSIHWVRQA |
| | | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVDVSHEDPEVKFNWYVDGVEVHNAKTKPREEQYN |
| | | STYRVVSVLTVLHQDWLNGKEYKCKVSNKALPASIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GSGGGSEVQLVESGGGLVQTGDSLRLSCEVSGRTFSSYSM |
| | | GWFRQAQGKEREFVVAISKGGYKYDSVSLEGRFTISKDNA |
| | | KNTVYLQINSLKPEDTAVYYCASSRAYGSSRLRLADTYEY |
| | | WGQGTQVTVSSGGGGSGGGSEVQLVESGGGLVQPGGSLRL |
| | | SCVASGIRFMSMAWYRQAPGKHRELVARISSGGTTAYVDS |
| | | VKGRFTISRDNSKNTVYLQMNSLKAEDTAVYYCNTFSSRP |
| | | NPWGAGTQVTVSS |
| 78 | 3.6 Knob HC | EVQLVESGGGLVQPGGSLRLSCAASGFDFTAYAMHWVRQA |
| | | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVDVSHEDPEVKFNWYVDGVEVHNAKTKPRE |
| | | EQYNSTYRVVSVLTVLHQDWLNGKEYKCKVSNKALPASIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGSGGGSEVQLVESGGGLVQTGDSLRLSCEVSGRTF |
| | | SSYSMGWFRQAQGKEREFVVAISKGGYKYDSVSLEGRFTI |
| | | SKDNAKNTVYLQINSLKPEDTAVYYCASSRAYGSSRLRLA |
| | | DTYEYWGQGTQVTVSSGGGGSGGGSEVQLVESGGGLVQPG |
| | | GSLRLSCVASGIRFMSMAWYRQAPGKHRELVARISSGGTT |
| | | AYVDSVKGRFTISRDNSKNTVYLQMNSLKAEDTAVYYCNT |
| | | FSSRPNPWGAGTQVTVSS |
| 79 | 3.6 LC | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGSEVQLVESGGGLVQTGDS |
| | | LRLSCEVSGRTFSSYSMGWFRQAQGKEREFVVAISKGGYK |
| | | YDSVSLEGRFTISKDNAKNTVYLQINSLKPEDTAVYYCAS |
| | | SRAYGSSRLRLADTYEYWGQGTQVTVSSGGGGSGGGSEVQ |
| | | LVESGGGLVQPGGSLRLSCVASGIRFMSMAWYRQAPGKHR |
| | | ELVARISSGGTTAYVDSVKGRFTISRDNSKNTVYLQMNSL |
| | | KAEDTAVYYCNTFSSRPNPWGAGTQVTVSS |
| 80 | 2.2 LC | DIQMTQSPSSLSASVGDRVTITCRASQSVSSAVAWYQQKP |
| | | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGGSGGGGSSDTGRPFVEMY |
| | | SEIPEIIHMTEGRELVIPCRVTSPNITVTLKKFPLDTLIP |
| | | DGKRIIWDSRKGFIISNATYKEIGLLTCEATVNGHLYKTN |
| | | YLTHRQTNTIIDVVLSPSHGIELSVGEKLVLNCTARTELN |
| | | VGIDENWEYPSSKHQHKKLVNRDLKTQSGSEMKKFLSTLT |
| | | IDGVTRSDQGLYTCAASSGLMTKKNSTFVRVHEK |
| 81 | 2.2 LC (Linker | DIQMTQSPSSLSASVGDRVTITCRASQSVSSAVAWYQQKP |
| | SEQ ID NO: | GKAPKLLIYSASSLYSGVPSRFSGSRSGTDFTLTISSLQP |
| | 33) | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGGSEPKSSDKTHTSDTGRP |
| | | FVEMYSEIPEIIHMTEGRELVIPCRVTSPNITVTLKKFP |
| | | LDTLIPDGKRIIWDSRKGFIISNATYKEIGLLTCEATVNG |
| | | HLYKTNYLTHRQTNTIIDVVLSPSHGIELSVGEKLVLNCT |
| | | ARTELNVGIDFNWEYPSSKHQHKKLVNRDLKTQSGSEMKK |
| | | FLSTLTIDGVTRSDQGLYTCAASSGLMTKKNSTFVRVHE |
| | | K |
| 82 | VHH1 + VHH2 | EVQLVESGGGLVQTGDSLRLSCEVSGRTFSSYSMGWFRQA |
| | | QGKEREFVVAISKGGYKYDSVSLEGRFTISKDNAKNTVYL |
| | | QINSLKPEDTAVYYCASSRAYGSSRLRLADTYEYWGQGTQ |
| | | VTVSSGGGGSGGGSEVQLVESGGGLVQPGGSLRLSCVASG |
| | | IRFMSMAWYRQAPGKHRELVARISSGGTTAYVDSVKGRFT |
| | | ISRDNSKNTVYLQMNSLKAEDTAVYYCNTFSSRPNPWGAG |
| | | TQVTVSS |
| 83 | VHH2 + VHH1 | EVQLVESGGGLVQPGGSLRLSCVASGIRFMSMAWYRQAPG |
| | | KHRELVARISSGGTTAYVDSVKGRFTISRDNSKNTVYLQM |
| | | NSLKAEDTAVYYCNTFSSRPNPWGAGTQVTVSSGGGGSGG |
| | | GSEVQLVESGGGLVQTGDSLRLSCEVSGRTFSSYSMGWFR |
| | | QAQGKEREFVVAISKGGYKYDSVSLEGRFTISKDNAKNTV |
| | | YLQINSLKPEDTAVYYCASSRAYGSSRLRLADTYEYWGQG |
| | | TQVTVSS |
| 84 | Signal Peptide 2 | MGWSCIILFLVATATGVHS |
| 85 | Signal Peptide 3 | METDTLLLWVLLLWVPGSTG |
| 86 | Signal Peptide 4 | MEWSWVFLFFLSVTTGVHS |
| 87 | DAB030448 | EVQLVESGGGLVQPGGSLRLSCAASGFDFSSSSIHWVRQA |
| | Hole HC | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVSVSHEDPEVKFNWYVDGVEVHNAKTKPREEQYN |
| | | STYRVVSVLTVLHQDWLNGKEYKCKVSNKALPAPIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GAGGGGAGGGGAGGGGASDTGRPFVEMYSEIPEIIHMTEG |
| | | RELVIPCRVTSPNITVTLKKFPLDTLIPDGKRIIWDSRKG |
| | | FIISNATYKEIGLLTCEATVNGHLYKTNYLTHRQTNTIID |
| | | VVLSPSHGIELSVGEKLVLNCTARTELNVGIDENWEYPSS |
| | | KHQHKKLVNRDLKTQSGSEMKKFLSTLTIDGVTRSDQGLY |
| | | TCAASSGLMTKKNSTFVRVHEK |
| 88 | DAB030448 | EVQLVESGGGLVQPGGSLRLSCAASGFDFTAYAMHWVRQA |
| | Knob HC | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVSVSHEDPEVKFNWYVDGVEVHNAKTKPRE |
| | | EQYNSTYRVVSVLTVLHQDWLNGKEYKCKVSNKALPAPIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGAGGGGAGGGGAGGGGASDTGRPFVEMYSEIPEII |
| | | HMTEGRELVIPCRVTSPNITVTLKKFPLDTLIPDGKRIIW |
| | | DSRKGFIISNATYKEIGLLTCEATVNGHLYKTNYLTHRQT |
| | | NTIIDVVLSPSHGIELSVGEKLVLNCTARTELNVGIDFNW |
| | | EYPSSKHQHKKLVNRDLKTQSGSEMKKFLSTLTIDGVTRS |
| | | DQGLYTCAASSGLMTKKNSTFVRVHEK |
| 89 | DAB030448 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 90 | DAB030450 | SDTGRPFVEMYSEIPEIIHMTEGRELVIPCRVTSPNITVT |
| | Hole HC | LKKFPLDTLIPDGKRIIWDSRKGFIISNATYKEIGLLTCE |
| | | ATVNGHLYKTNYLTHRQTNTIIDVVLSPSHGIELSVGEKL |
| | | VLNCTARTELNVGIDENWEYPSSKHQHKKLVNRDLKTQSG |
| | | SEMKKFLSTLTIDGVTRSDQGLYTCAASSGLMTKKNSTFV |
| | | RVHEKGGGGAGGGGAGGGGAGGGGAEVQLVESGGGLVQP |
| | | GGSLRLSCAASGFDFSSSSIHWVRQAPGKGLEWVASISSS |
| | | YGYTYYADSVKGRFTISADTSKNTAYLQMNSLRAEDTAVY |
| | | YCARSWAMDYWGQGTLVTVSSGGGGSDIQMTQSPSSLSAS |
| | | VGDRVTITCRASQSVSSAVAWYQQKPGKAPKLLIYSASDL |
| | | YSGVPSRFSGSRSGTDFTLTISSLQPEDFATYYCQQYAGA |
| | | GLITFGQGTKVEIKGGGGSGGGGSEPKSSDKTHTCPPCPA |
| | | PEAAGGPSVFLFPPKPKDTLMISRTPEVTCVVVSVSHEDP |
| | | EVKFNWYVDGVEVHNAKTKPREEQYNSTYRVVSVLTVLHQ |
| | | DWLNGKEYKCKVSNKALPAPIEKTISKAKGQPREPQVYTL |
| | | PPIRELMTSNQVSLSCAVKGFYPSDIAVEWESNGQPENN |
| | | YKTTPPVLDSDGSFFLVSKLTVDKSRWQQGNVFSCSVMHE |
| | | ALHNHYTQKSLSLSPGKGGGGSGGGGSEPKSSDKTHTEVQ |
| | | LVESGGGLVQPGGSLRLSCAASGFTLSSYSMHWVRQAPGK |
| | | GLEWVAYISSYDSITDYADSVKGRFTISADTSKNTAYLQM |
| | | NSLRAEDTAVYYCARPAVGHMAFDYWGQGTLVTVSSASTK |
| | | GPSVFPLAPSSKSTSGGTAALGCLVKDYFPEPVTVSWNSG |
| | | ALTSGVHTFPAVLQSSGLYSLSSVVTVPSSSLGTQTYICN |
| | | VNHKPSNTKVDKKVEPKSCDKTHT |
| 91 | DAB030450 | SDTGRPFVEMYSEIPEIIHMTEGRELVIPCRVTSPNITV |
| | Knob HC | TLKKFPLDTLIPDGKRIIWDSRKGFIISNATYKEIGLLTC |
| | | EATVNGHLYKTNYLTHRQTNTIIDVVLSPSHGIELSVGEK |
| | | LVLNCTARTELNVGIDENWEYPSSKHQHKKLVNRDLKTQS |
| | | GSEMKKFLSTLTIDGVTRSDQGLYTCAASSGLMTKKNSTF |
| | | VRVHEKGGGGAGGGGAGGGGAGGGGAEVQLVESGGGLVQ |
| | | PGGSLRLSCAASGFDFTAYAMHWVRQAPGKGLEWVASIYP |
| | | SGGYTAYADSVKGRFTISADTSKNTAYLQMNSLRAEDTAV |
| | | YYCARRSYYFALDYWGQGTLVTVSSGGGGSDIQMTQSPSS |
| | | LSASVGDRVTITCRASQSVSSAVAWYQQKPGKAPKLLIYS |
| | | ASSLYSGVPSRFSGSRSGTDFTLTISSLQPEDFATYYCQQ |
| | | YWAYYSPITFGQGTKVEIKGGGGSGGGGSEPKSSDKTHTC |
| | | PPCPAPEAAGGPSVFLFPPKPKDTLMISRTPEVTCVVVSV |
| | | SHEDPEVKFNWYVDGVEVHNAKTKPREEQYNSTYRVVSVL |
| | | TVLHQDWLNGKEYKCKVSNKALPAPIEKTISKAKGQPREP |
| | | MVFDLPPSREEMTKNQVSLWCMVKGFYPSDIAVEWESNG |
| | | QPENNYKTTPPVLDSDGSFFLYSKLTVDKSRWQQGNVFSC |
| | | SVMHEALHNHYTQKSLSLSPGKGGGGSGGGGSEPKSSDKT |
| | | HTEVQLVESGGGLVQPGGSLRLSCAASGFTLSSYSMHWVR |
| | | QAPGKGLEWVAYISSYDSITDYADSVKGRFTISADTSKNT |
| | | AYLQMNSLRAEDTAVYYCARPAVGHMAFDYWGQGTLVTVS |
| | | SASTKGPSVFPLAPSSKSTSGGTAALGCLVKDYFPEPVTV |
| | | SWNSGALTSGVHTFPAVLQSSGLYSLSSVVTVPSSSLGTQ |
| | | TYICNVNHKPSNTKVDKKVEPKSCDKTHT |
| 92 | DAB030450 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 93 | DAB030452 | EVQLVESGGGLVQPGGSLRLSCAASGFDFSSSSIHWVRQA |
| | Hole HC | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVSVSHEDPEVKFNWYVDGVEVHNAKTKPREEQYN |
| | | STYRVVSVLTVLHQDWLNGKEYKCKVSNKALPAPIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GAGGGGAGGGGAGGGGASDTGRPFVEMYSEIPEIIHMTEG |
| | | RELVIPCRVTSPNITVTLKKFPLDTLIPDGKRIIWDSRKG |
| | | FIISNATYKEIGLLTCEATVNGHLYKTNYLTHRQTNTIID |
| | | VVLSPSHGIELSVGEKLVLNCTARTELNVGIDENWEYPSS |
| | | KHQHKKLVNRDLKTQSGSEMKKFLSTLTIDGVTRSDQGLY |
| | | TCAASSGLMTKKNSTFVRVHEK |
| 94 | DAB030452 | EVQLVESGGGLVQPGGSLRLSCAASGFDFTAYAMHWVRQA |
| | Knob HC | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVSVSHEDPEVKFNWYVDGVEVHNAKTKPRE |
| | | EQYNSTYRVVSVLTVLHQDWLNGKEYKCKVSNKALPAPIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGAGGGGAGGGGAGGGGASDTGRPFVEMYSEIPEII |
| | | HMTEGRELVIPCRVTSPNITVTLKKFPLDTLIPDGKRIIW |
| | | DSRKGFIISNATYKEIGLLTCEATVNGHLYKTNYLTHRQT |
| | | NTIIDVVLSPSHGIELSVGEKLVLNCTARTELNVGIDFNW |
| | | EYPSSKHQHKKLVNRDLKTQSGSEMKKFLSTLTIDGVTRS |
| | | DQGLYTCAASSGLMTKKNSTFVRVHEK |
| 95 | DAB030452 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGAGGGGAGGGGAGGGGASDTGRP |
| | | FVEMYSEIPEIIHMTEGRELVIPCRVTSPNITVTLKKFPL |
| | | DTLIPDGKRIIWDSRKGFIISNATYKEIGLLTCEATVNGH |
| | | LYKTNYLTHRQTNTIIDVVLSPSHGIELSVGEKLVLNCTA |
| | | RTELNVGIDFNWEYPSSKHQHKKLVNRDLKTQSGSEMKKF |
| | | LSTLTIDGVTRSDQGLYTCAASSGLMTKKNSTFVRVHEK |
| 96 | DAB032370 | EVQLVESGGGLVQPGGSLRLSCAASGFDFSSSSIHWVRQA |
| | Hole HC | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVSVSHEDPEVKFNWYVDGVEVHNAKTKPREEQYN |
| | | STYRVVSVLTVLHQDWLNGKEYKCKVSNKALPAPIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GSGGGGSGGGGSGGGGSSDTGRPFVEMYSEIPEIIHMTEG |
| | | RELVIPCRVTSPNITVTLKKFPLDTLIPDGKRIIWDSRKG |
| | | FIISNATYKEIGLLTCEATVNGHLYKTNYLTHRQTNTIID |
| | | VVLSPSHGIELSVGEKLVLNCTARTELNVGIDENWEYPSS |
| | | KHQHKKLVNRDLKTQSGSEMKKFLSTLTIDGVTRSDQGLY |
| | | TCAASSGLMTKKNSTFVRVHEK |
| 97 | DAB032370 | EVQLVESGGGLVQPGGSLRLSCAASGFDFTAYAMHWVRQA |
| | Knob HC | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVSVSHEDPEVKFNWYVDGVEVHNAKTKPRE |
| | | EQYNSTYRVVSVLTVLHQDWLNGKEYKCKVSNKALPAPIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGSGGGGSGGGGSGGGGSSDTGRPFVEMYSEIPEII |
| | | HMTEGRELVIPCRVTSPNITVTLKKFPLDTLIPDGKRIIW |
| | | DSRKGFIISNATYKEIGLLTCEATVNGHLYKTNYLTHRQT |
| | | NTIIDVVLSPSHGIELSVGEKLVLNCTARTELNVGIDENW |
| | | EYPSSKHQHKKLVNRDLKTQSGSEMKKFLSTLTIDGVTRS |
| | | DQGLYTCAASSGLMTKKNSTFVRVHEK |
| 98 | DAB032370 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 99 | DAB032367 | SDTGRPFVEMYSEIPEIIHMTEGRELVIPCRVTSPNITVT |
| | Hole HC | LKKFPLDTLIPDGKRIIWDSRKGFIISNATYKEIGLLTCE |
| | | ATVNGHLYKTNYLTHRQTNTIIDVVLSPSHGIELSVGEKL |
| | | VLNCTARTELNVGIDENWEYPSSKHQHKKLVNRDLKTQSG |
| | | SEMKKFLSTLTIDGVTRSDQGLYTCAASSGLMTKKNSTFV |
| | | RVHEKGGGGSGGGGSGGGGSGGGGSEVQLVESGGGLVQPG |
| | | GSLRLSCAASGFDFSSSSIHWVRQAPGKGLEWVASISSSY |
| | | GYTYYADSVKGRFTISADTSKNTAYLQMNSLRAEDTAVYY |
| | | CARSWAMDYWGQGTLVTVSSGGGGSDIQMTQSPSSLSASV |
| | | GDRVTITCRASQSVSSAVAWYQQKPGKAPKLLIYSASDLY |
| | | SGVPSRFSGSRSGTDFTLTISSLQPEDFATYYCQQYAGAG |
| | | LITFGQGTKVEIKGGGGSGGGGSEPKSSDKTHTCPPCPAP |
| | | EAAGGPSVFLFPPKPKDTLMISRTPEVTCVVVSVSHEDPE |
| | | VKFNWYVDGVEVHNAKTKPREEQYNSTYRVVSVLTVLHQD |
| | | WLNGKEYKCKVSNKALPAPIEKTISKAKGQPREPQVYTLP |
| | | PIRELMTSNQVSLSCAVKGFYPSDIAVEWESNGQPENNY |
| | | KTTPPVLDSDGSFFLVSKLTVDKSRWQQGNVFSCSVMHEA |
| | | LHNHYTQKSLSLSPGKGGGGSGGGGSEPKSSDKTHTEVQL |
| | | VESGGGLVQPGGSLRLSCAASGFTLSSYSMHWVRQAPGKG |
| | | LEWVAYISSYDSITDYADSVKGRFTISADTSKNTAYLQMN |
| | | SLRAEDTAVYYCARPAVGHMAFDYWGQGTLVTVSSASTKG |
| | | PSVFPLAPSSKSTSGGTAALGCLVKDYFPEPVTVSWNSGA |
| | | LTSGVHTFPAVLQSSGLYSLSSVVTVPSSSLGTQTYICNV |
| | | NHKPSNTKVDKKVEPKSCDKTHT |
| 100 | DAB032367 | SDTGRPFVEMYSEIPEIIHMTEGRELVIPCRVTSPNITV |
| | Knob HC | TLKKFPLDTLIPDGKRIIWDSRKGFIISNATYKEIGLLTC |
| | | EATVNGHLYKTNYLTHRQTNTIIDVVLSPSHGIELSVGEK |
| | | LVLNCTARTELNVGIDENWEYPSSKHQHKKLVNRDLKTQS |
| | | GSEMKKFLSTLTIDGVTRSDQGLYTCAASSGLMTKKNSTF |
| | | VRVHEKGGGGSGGGGSGGGGSGGGGSEVQLVESGGGLVQP |
| | | GGSLRLSCAASGFDFTAYAMHWVRQAPGKGLEWVASIYPS |
| | | GGYTAYADSVKGRFTISADTSKNTAYLQMNSLRAEDTAVY |
| | | YCARRSYYFALDYWGQGTLVTVSSGGGGSDIQMTQSPSSL |
| | | SASVGDRVTITCRASQSVSSAVAWYQQKPGKAPKLLIYSA |
| | | SSLYSGVPSRFSGSRSGTDFTLTISSLQPEDFATYYCQQY |
| | | WAYYSPITFGQGTKVEIKGGGGSGGGGSEPKSSDKTHTCP |
| | | PCPAPEAAGGPSVFLFPPKPKDTLMISRTPEVTCVVVSVS |
| | | HEDPEVKFNWYVDGVEVHNAKTKPREEQYNSTYRVVSVLT |
| | | VLHQDWLNGKEYKCKVSNKALPAPIEKTISKAKGQPREPM |
| | | VFDLPPSREEMTKNQVSLWCMVKGFYPSDIAVEWESNGQP |
| | | ENNYKTTPPVLDSDGSFFLYSKLTVDKSRWQQGNVFSCSV |
| | | MHEALHNHYTQKSLSLSPGKGGGGSGGGGSEPKSSDKTHT |
| | | EVQLVESGGGLVQPGGSLRLSCAASGFTLSSYSMHWVRQA |
| | | PGKGLEWVAYISSYDSITDYADSVKGRFTISADTSKNTAY |
| | | LQMNSLRAEDTAVYYCARPAVGHMAFDYWGQGTLVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSCDKTHT |
| 101 | DAB032367 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 102 | DAB032364 | EVQLVESGGGLVQPGGSLRLSCAASGFDFSSSSIHWVRQA |
| | Hole HC | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVSVSHEDPEVKFNWYVDGVEVHNAKTKPREEQYN |
| | | STYRVVSVLTVLHQDWLNGKEYKCKVSNKALPAPIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHTGGG |
| | | GSGGGGSGGGGSGGGGSSDTGRPFVEMYSEIPEIIHMTEG |
| | | RELVIPCRVTSPNITVTLKKFPLDTLIPDGKRIIWDSRKG |
| | | FIISNATYKEIGLLTCEATVNGHLYKTNYLTHRQTNTIID |
| | | VVLSPSHGIELSVGEKLVLNCTARTELNVGIDENWEYPSS |
| | | KHQHKKLVNRDLKTQSGSEMKKFLSTLTIDGVTRSDQGLY |
| | | TCAASSGLMTKKNSTFVRVHEK |
| 103 | DAB032364 | EVQLVESGGGLVQPGGSLRLSCAASGFDFTAYAMHWVRQA |
| | Knob HC | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVSVSHEDPEVKFNWYVDGVEVHNAKTKPRE |
| | | EQYNSTYRVVSVLTVLHQDWLNGKEYKCKVSNKALPAPIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HTGGGGSGGGGSGGGGSGGGGSSDTGRPFVEMYSEIPEII |
| | | HMTEGRELVIPCRVTSPNITVTLKKFPLDTLIPDGKRIIW |
| | | DSRKGFIISNATYKEIGLLTCEATVNGHLYKTNYLTHRQT |
| | | NTIIDVVLSPSHGIELSVGEKLVLNCTARTELNVGIDENW |
| | | EYPSSKHQHKKLVNRDLKTQSGSEMKKFLSTLTIDGVTRS |
| | | DQGLYTCAASSGLMTKKNSTFVRVHEK |
| 104 | DAB032364 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGGSGGGGSGGGGSSDTGRP |
| | | FVEMYSEIPEIIHMTEGRELVIPCRVTSPNITVTLKKFP |
| | | LDTLIPDGKRIIWDSRKGFIISNATYKEIGLLTCEATVNG |
| | | HLYKTNYLTHRQTNTIIDVVLSPSHGIELSVGEKLVLNCT |
| | | ARTELNVGIDFNWEYPSSKHQHKKLVNRDLKTQSGSEMKK |
| | | FLSTLTIDGVTRSDQGLYTCAASSGLMTKKNSTFVRVHE |
| | | K |
| 105 | DAB032365 | SDTGRPFVEMYSEIPEIIHMTEGRELVIPCRVTSPNIT |
| | Hole HC | VTLKKFPLDTLIPDGKRIIWDSRKGFIISNATYKEIGLLT |
| | | CEATVNGHLYKTNYLTHRQTNTIIDVVLSPSHGIELSVGE |
| | | KLVLNCTARTELNVGIDENWEYPSSKHQHKKLVNRDLKTQ |
| | | SGSEMKKFLSTLTIDGVTRSDQGLYTCAASSGLMTKKNST |
| | | FVRVHEKGGGGSGGGGSEVQLVESGGGLVQPGGSLRLSC |
| | | AASGFDFSSSSIHWVRQAPGKGLEWVASISSSYGYTYYAD |
| | | SVKGRFTISADTSKNTAYLQMNSLRAEDTAVYYCARSWAM |
| | | DYWGQGTLVTVSSGGGGSDIQMTQSPSSLSASVGDRVTIT |
| | | CRASQSVSSAVAWYQQKPGKAPKLLIYSASDLYSGVPSRF |
| | | SGSRSGTDFTLTISSLQPEDFATYYCQQYAGAGLITFGQG |
| | | TKVEIKGGGGSGGGGSEPKSSDKTHTCPPCPAPEAAGGPS |
| | | VFLFPPKPKDTLMISRTPEVTCVVVSVSHEDPEVKFNWYV |
| | | DGVEVHNAKTKPREEQYNSTYRVVSVLTVLHQDWLNGKEY |
| | | KCKVSNKALPAPIEKTISKAKGQPREPQVYTLPPIRELMT |
| | | SNQVSLSCAVKGFYPSDIAVEWESNGQPENNYKTTPPVL |
| | | DSDGSFFLVSKLTVDKSRWQQGNVFSCSVMHEALHNHYTQ |
| | | KSLSLSPGKGGGGSGGGGSEPKSSDKTHTEVQLVESGGGL |
| | | VQPGGSLRLSCAASGFTLSSYSMHWVRQAPGKGLEWVAYI |
| | | SSYDSITDYADSVKGRFTISADTSKNTAYLQMNSLRAEDT |
| | | AVYYCARPAVGHMAFDYWGQGTLVTVSSASTKGPSVFPLA |
| | | PSSKSTSGGTAALGCLVKDYFPEPVTVSWNSGALTSGVHT |
| | | FPAVLQSSGLYSLSSVVTVPSSSLGTQTYICNVNHKPSNT |
| | | KVDKKVEPKSCDKTHT |
| 106 | DAB032365 | SDTGRPFVEMYSEIPEIIHMTEGRELVIPCRVTSPNITV |
| | Knob HC | TLKKFPLDTLIPDGKRIIWDSRKGFIISNATYKEIGLLTC |
| | | EATVNGHLYKTNYLTHRQTNTIIDVVLSPSHGIELSVGEK |
| | | LVLNCTARTELNVGIDENWEYPSSKHQHKKLVNRDLKTQS |
| | | GSEMKKFLSTLTIDGVTRSDQGLYTCAASSGLMTKKNSTF |
| | | VRVHEKGGGGSGGGGSEVQLVESGGGLVQPGGSLRLSCA |
| | | ASGFDFTAYAMHWVRQAPGKGLEWVASIYPSGGYTAYADS |
| | | VKGRFTISADTSKNTAYLQMNSLRAEDTAVYYCARRSYYF |
| | | ALDYWGQGTLVTVSSGGGGSDIQMTQSPSSLSASVGDRVT |
| | | ITCRASQSVSSAVAWYQQKPGKAPKLLIYSASSLYSGVPS |
| | | RFSGSRSGTDFTLTISSLQPEDFATYYCQQYWAYYSPITF |
| | | GQGTKVEIKGGGGSGGGGSEPKSSDKTHTCPPCPAPEAAG |
| | | GPSVFLFPPKPKDTLMISRTPEVTCVVVSVSHEDPEVKFN |
| | | WYVDGVEVHNAKTKPREEQYNSTYRVVSVLTVLHQDWLNG |
| | | KEYKCKVSNKALPAPIEKTISKAKGQPREPMVFDLPPSRE |
| | | EMTKNQVSLWCMVKGFYPSDIAVEWESNGQPENNYKTTP |
| | | PVLDSDGSFFLYSKLTVDKSRWQQGNVFSCSVMHEALHNH |
| | | YTQKSLSLSPGKGGGGSGGGGSEPKSSDKTHTEVQLVESG |
| | | GGLVQPGGSLRLSCAASGFTLSSYSMHWVRQAPGKGLEWV |
| | | AYISSYDSITDYADSVKGRFTISADTSKNTAYLQMNSLRA |
| | | EDTAVYYCARPAVGHMAFDYWGQGTLVTVSSASTKGPSVF |
| | | PLAPSSKSTSGGTAALGCLVKDYFPEPVTVSWNSGALTSG |
| | | VHTFPAVLQSSGLYSLSSVVTVPSSSLGTQTYICNVNHKP |
| | | SNTKVDKKVEPKSCDKTHT |
| 107 | DAB032365 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 108 | DAB032366 | SDTGRPFVEMYSEIPEIIHMTEGRELVIPCRVTSPNITV |
| | Hole HC | TLKKFPLDTLIPDGKRIIWDSRKGFIISNATYKEIGLLTC |
| | | EATVNGHLYKTNYLTHRQTNTIIDVVLSPSHGIELSVGEK |
| | | LVLNCTARTELNVGIDENWEYPSSKHQHKKLVNRDLKTQS |
| | | GSEMKKFLSTLTIDGVTRSDQGLYTCAASSGLMTKKNSTF |
| | | VRVHEKGGGGSGGGGSGGGGSEVQLVESGGGLVQPGGSL |
| | | RLSCAASGFDFSSSSIHWVRQAPGKGLEWVASISSSYGYT |
| | | YYADSVKGRFTISADTSKNTAYLQMNSLRAEDTAVYYCAR |
| | | SWAMDYWGQGTLVTVSSGGGGSDIQMTQSPSSLSASVGDR |
| | | VTITCRASQSVSSAVAWYQQKPGKAPKLLIYSASDLYSGV |
| | | PSRFSGSRSGTDFTLTISSLQPEDFATYYCQQYAGAGLIT |
| | | FGQGTKVEIKGGGGSGGGGSEPKSSDKTHTCPPCPAPEAA |
| | | GGPSVFLFPPKPKDTLMISRTPEVTCVVVSVSHEDPEVKF |
| | | NWYVDGVEVHNAKTKPREEQYNSTYRVVSVLTVLHQDWLN |
| | | GKEYKCKVSNKALPAPIEKTISKAKGQPREPQVYTLPPIR |
| | | ELMTSNQVSLSCAVKGFYPSDIAVEWESNGQPENNYKTTP |
| | | PVLDSDGSFFLVSKLTVDKSRWQQGNVFSCSVMHEALHNH |
| | | YTQKSLSLSPGKGGGGSGGGGSEPKSSDKTHTEVQLVESG |
| | | GGLVQPGGSLRLSCAASGFTLSSYSMHWVRQAPGKGLEWV |
| | | AYISSYDSITDYADSVKGRFTISADTSKNTAYLQMNSLRA |
| | | EDTAVYYCARPAVGHMAFDYWGQGTLVTVSSASTKGPSVF |
| | | PLAPSSKSTSGGTAALGCLVKDYFPEPVTVSWNSGALTSG |
| | | VHTFPAVLQSSGLYSLSSVVTVPSSSLGTQTYICNVNHKP |
| | | SNTKVDKKVEPKSCDKTHT |
| 109 | DAB032366 | SDTGRPFVEMYSEIPEIIHMTEGRELVIPCRVTSPNIT |
| | Knob HC | VTLKKFPLDTLIPDGKRIIWDSRKGFIISNATYKEIGLLT |
| | | CEATVNGHLYKTNYLTHRQTNTIIDVVLSPSHGIELSVGE |
| | | KLVLNCTARTELNVGIDFNWEYPSSKHQHKKLVNRDLKTQ |
| | | SGSEMKKFLSTLTIDGVTRSDQGLYTCAASSGLMTKKNST |
| | | FVRVHEKGGGGSGGGGSGGGGSEVQLVESGGGLVQPGGS |
| | | LRLSCAASGFDFTAYAMHWVRQAPGKGLEWVASIYPSGGY |
| | | TAYADSVKGRFTISADTSKNTAYLQMNSLRAEDTAVYYCA |
| | | RRSYYFALDYWGQGTLVTVSSGGGGSDIQMTQSPSSLSAS |
| | | VGDRVTITCRASQSVSSAVAWYQQKPGKAPKLLIYSASSL |
| | | YSGVPSRFSGSRSGTDFTLTISSLQPEDFATYYCQQYWAY |
| | | YSPITFGQGTKVEIKGGGGSGGGGSEPKSSDKTHTCPPCP |
| | | APEAAGGPSVFLFPPKPKDTLMISRTPEVTCVVVSVSHED |
| | | PEVKFNWYVDGVEVHNAKTKPREEQYNSTYRVVSVLTVLH |
| | | QDWLNGKEYKCKVSNKALPAPIEKTISKAKGQPREPMVFD |
| | | LPPSREEMTKNQVSLWCMVKGFYPSDIAVEWESNGQPENN |
| | | YKTTPPVLDSDGSFFLYSKLTVDKSRWQQGNVFSCSVMHE |
| | | ALHNHYTQKSLSLSPGKGGGGSGGGGSEPKSSDKTHTEVQ |
| | | LVESGGGLVQPGGSLRLSCAASGFTLSSYSMHWVRQAPGK |
| | | GLEWVAYISSYDSITDYADSVKGRFTISADTSKNTAYLQM |
| | | NSLRAEDTAVYYCARPAVGHMAFDYWGQGTLVTVSSASTK |
| | | GPSVFPLAPSSKSTSGGTAALGCLVKDYFPEPVTVSWNSG |
| | | ALTSGVHTFPAVLQSSGLYSLSSVVTVPSSSLGTQTYICN |
| | | VNHKPSNTKVDKKVEPKSCDKTHT |
| 110 | DAB032366 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 111 | DAB030449 | SDTGRPFVEMYSEIPEIIHMTEGRELVIPCRVTSPNITV |
| | Hole HC | TLKKFPLDTLIPDGKRIIWDSRKGFIISNATYKEIGLLTC |
| | | EATVNGHLYKTNYLTHRQTNTIIDVVLSPSHGIELSVGEK |
| | | LVLNCTARTELNVGIDENWEYPSSKHQHKKLVNRDLKTQS |
| | | GSEMKKFLSTLTIDGVTRSDQGLYTCAASSGLMTKKNSTF |
| | | VRVHEKGGGGAGGGGAEVQLVESGGGLVQPGGSLRLSCA |
| | | ASGFDFSSSSIHWVRQAPGKGLEWVASISSSYGYTYYADS |
| | | VKGRFTISADTSKNTAYLQMNSLRAEDTAVYYCARSWAMD |
| | | YWGQGTLVTVSSGGGGSDIQMTQSPSSLSASVGDRVTITC |
| | | RASQSVSSAVAWYQQKPGKAPKLLIYSASDLYSGVPSRFS |
| | | GSRSGTDFTLTISSLQPEDFATYYCQQYAGAGLITFGQGT |
| | | KVEIKGGGGSGGGGSEPKSSDKTHTCPPCPAPEAAGGPSV |
| | | FLFPPKPKDTLMISRTPEVTCVVVSVSHEDPEVKFNWYVD |
| | | GVEVHNAKTKPREEQYNSTYRVVSVLTVLHQDWLNGKEYK |
| | | CKVSNKALPAPIEKTISKAKGQPREPQVYTLPPIRELMTS |
| | | NQVSLSCAVKGFYPSDIAVEWESNGQPENNYKTTPPVLD |
| | | SDGSFFLVSKLTVDKSRWQQGNVFSCSVMHEALHNHYTQK |
| | | SLSLSPGKGGGGSGGGGSEPKSSDKTHTEVQLVESGGGLV |
| | | QPGGSLRLSCAASGFTLSSYSMHWVRQAPGKGLEWVAYIS |
| | | SYDSITDYADSVKGRFTISADTSKNTAYLQMNSLRAEDTA |
| | | VYYCARPAVGHMAFDYWGQGTLVTVSSASTKGPSVFPLAP |
| | | SSKSTSGGTAALGCLVKDYFPEPVTVSWNSGALTSGVHTF |
| | | PAVLQSSGLYSLSSVVTVPSSSLGTQTYICNVNHKPSNTK |
| | | VDKKVEPKSCDKTHT |
| 112 | DAB030449 | SDTGRPFVEMYSEIPEIIHMTEGRELVIPCRVTSPNITV |
| | Knob HC | TLKKFPLDTLIPDGKRIIWDSRKGFIISNATYKEIGLLTC |
| | | EATVNGHLYKTNYLTHRQTNTIIDVVLSPSHGIELSVGEK |
| | | LVLNCTARTELNVGIDENWEYPSSKHQHKKLVNRDLKTQS |
| | | GSEMKKFLSTLTIDGVTRSDQGLYTCAASSGLMTKKNSTF |
| | | VRVHEKGGGGAGGGGAEVQLVESGGGLVQPGGSLRLSCA |
| | | ASGFDFTAYAMHWVRQAPGKGLEWVASIYPSGGYTAYADS |
| | | VKGRFTISADTSKNTAYLQMNSLRAEDTAVYYCARRSYYF |
| | | ALDYWGQGTLVTVSSGGGGSDIQMTQSPSSLSASVGDRVT |
| | | ITCRASQSVSSAVAWYQQKPGKAPKLLIYSASSLYSGVPS |
| | | RFSGSRSGTDFTLTISSLQPEDFATYYCQQYWAYYSPITF |
| | | GQGTKVEIKGGGGSGGGGSEPKSSDKTHTCPPCPAPEAAG |
| | | GPSVFLFPPKPKDTLMISRTPEVTCVVVSVSHEDPEVKFN |
| | | WYVDGVEVHNAKTKPREEQYNSTYRVVSVLTVLHQDWLNG |
| | | KEYKCKVSNKALPAPIEKTISKAKGQPREPMVFDLPPSRE |
| | | EMTKNQVSLWCMVKGFYPSDIAVEWESNGQPENNYKTTP |
| | | PVLDSDGSFFLYSKLTVDKSRWQQGNVFSCSVMHEALHNH |
| | | YTQKSLSLSPGKGGGGSGGGGSEPKSSDKTHTEVQLVESG |
| | | GGLVQPGGSLRLSCAASGFTLSSYSMHWVRQAPGKGLEWV |
| | | AYISSYDSITDYADSVKGRFTISADTSKNTAYLQMNSLRA |
| | | EDTAVYYCARPAVGHMAFDYWGQGTLVTVSSASTKGPSVF |
| | | PLAPSSKSTSGGTAALGCLVKDYFPEPVTVSWNSGALTSG |
| | | VHTFPAVLQSSGLYSLSSVVTVPSSSLGTQTYICNVNHKP |
| | | SNTKVDKKVEPKSCDKTHT |
| 113 | DAB030449 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 114 | DAB032372 | EVQLVESGGGLVQPGGSLRLSCAASGFDFSSSSIHWVRQA |
| | Hole HC | PGKGLEWVASISSSYGYTYYADSVKGRFTISADTSKNTAY |
| | | LQMNSLRAEDTAVYYCARSWAMDYWGQGTLVTVSSGGGGS |
| | | DIQMTQSPSSLSASVGDRVTITCRASQSVSSAVAWYQQKP |
| | | GKAPKLLIYSASDLYSGVPSRFSGSRSGTDFTLTISSLQP |
| | | EDFATYYCQQYAGAGLITFGQGTKVEIKGGGGSGGGGSEP |
| | | KSSDKTHTCPPCPAPEAAGGPSVFLFPPKPKDTLMISRTP |
| | | EVTCVVVSVSHEDPEVKFNWYVDGVEVHNAKTKPREEQYN |
| | | STYRVVSVLTVLHQDWLNGKEYKCKVSNKALPAPIEKTIS |
| | | KAKGQPREPQVYTLPPIRELMTSNQVSLSCAVKGFYPSDI |
| | | AVEWESNGQPENNYKTTPPVLDSDGSFFLVSKLTVDKSRW |
| | | QQGNVFSCSVMHEALHNHYTQKSLSLSPGKGGGGSGGGGS |
| | | EPKSSDKTHTEVQLVESGGGLVQPGGSLRLSCAASGFTLS |
| | | SYSMHWVRQAPGKGLEWVAYISSYDSITDYADSVKGRFTI |
| | | SADTSKNTAYLQMNSLRAEDTAVYYCARPAVGHMAFDYWG |
| | | QGTLVTVSSASTKGPSVFPLAPSSKSTSGGTAALGCLVKD |
| | | YFPEPVTVSWNSGALTSGVHTFPAVLQSSGLYSLSSVVTV |
| | | PSSSLGTQTYICNVNHKPSNTKVDKKVEPKSCDKTHT |
| 115 | DAB032372 | EVQLVESGGGLVQPGGSLRLSCAASGFDFTAYAMHWVRQA |
| | Knob HC | PGKGLEWVASIYPSGGYTAYADSVKGRFTISADTSKNTAY |
| | | LQMNSLRAEDTAVYYCARRSYYFALDYWGQGTLVTVSSGG |
| | | GGSDIQMTQSPSSLSASVGDRVTITCRASQSVSSAVAWYQ |
| | | QKPGKAPKLLIYSASSLYSGVPSRFSGSRSGTDFTLTISS |
| | | LQPEDFATYYCQQYWAYYSPITFGQGTKVEIKGGGGSGGG |
| | | GSEPKSSDKTHTCPPCPAPEAAGGPSVFLFPPKPKDTLMI |
| | | SRTPEVTCVVVSVSHEDPEVKFNWYVDGVEVHNAKTKPRE |
| | | EQYNSTYRVVSVLTVLHQDWLNGKEYKCKVSNKALPAPIE |
| | | KTISKAKGQPREPMVFDLPPSREEMTKNQVSLWCMVKGFY |
| | | PSDIAVEWESNGQPENNYKTTPPVLDSDGSFFLYSKLTV |
| | | DKSRWQQGNVFSCSVMHEALHNHYTQKSLSLSPGKGGGGS |
| | | GGGGSEPKSSDKTHTEVQLVESGGGLVQPGGSLRLSCAAS |
| | | GFTLSSYSMHWVRQAPGKGLEWVAYISSYDSITDYADSVK |
| | | GRFTISADTSKNTAYLQMNSLRAEDTAVYYCARPAVGHMA |
| | | FDYWGQGTLVTVSSASTKGPSVFPLAPSSKSTSGGTAALG |
| | | CLVKDYFPEPVTVSWNSGALTSGVHTFPAVLQSSGLYSLS |
| | | SVVTVPSSSLGTQTYICNVNHKPSNTKVDKKVEPKSCDKT |
| | | HT |
| 116 | DAB032372 | DIQMTQSPSSLSASVGDRVTITCRASQSVSSAVAWYQQKP |
| | LC | GKAPKLLIYSASSLYSGVPSRFSGSRSGTDFTLTISSLQP |
| | | EDFATYYCQQWYNAPITFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGECGGGGSGGGGSGGGGSGGGGSSDTGRP |
| | | FVEMYSEIPEIIHMTEGRELVIPCRVTSPNITVTLKKFP |
| | | LDTLIPDGKRIIWDSRKGFIISNATYKEIGLLTCEATVNG |
| | | HLYKTNYLTHRQTNTIIDVVLSPSHGIELSVGEKLVLNCT |
| | | ARTELNVGIDFNWEYPSSKHQHKKLVNRDLKTQSGSEMKK |
| | | FLSTLTIDGVTRSDQGLYTCAASSGLMTKKNSTFVRVHE |
| | | K |
| 117 | Linker | GGGGA |
| 118 | Linker | GGGGAGGGGAGGGGAGGGGA |
| 119 | Linker | GGGGAGGGGAGGGGA |
| 120 | Linker | GGGGSGGGGS |
| 121 | Linker | GGGGSGGGGSGGGGS |
| 122 | Linker | GGGGSGGGGSGGGGSGGGGS |

1. A multivalent antibody binding molecule, comprising an LRP5 binding domain that specifically binds low-density lipoprotein receptor-related protein 5 (LRP5), an Fc domain, an FZD4 binding domain that specifically binds Frizzled 4 (FZD4), and one or more VEGF binding domains that each specifically bind vascular endothelial growth factor (VEGF);
wherein the multivalent antibody binding molecule comprises four polypeptides, wherein:
(a) the first polypeptide comprises:
(i) a first VH comprising a CDR-H1 having the amino acid sequence of SEQ ID NO: 1, a CDR-H2 having the amino acid sequence of SEQ ID NO: 2, and a CDR-H3 having the amino acid sequence of SEQ ID NO: 3;
(ii) a VL comprising a CDR-L1 having the amino acid sequence of SEQ ID NO: 10, a CDR-L2 having the amino acid sequence of SEQ ID NO: 11, and a CDR-L3 having the amino acid sequence of SEQ ID NO: 12; and
(iii) a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37;
(b) the second polypeptide comprises:
(i) a first VH comprising a CDR-H1 having the amino acid sequence of SEQ ID NO: 7, a CDR-H2 having the amino acid sequence of SEQ ID NO: 8, and a CDR-H3 having the amino acid sequence of SEQ ID NO: 9;
(ii) a VL comprising a CDR-L1 having the amino acid sequence of SEQ ID NO: 4, a CDR-L2 having the amino acid sequence of SEQ ID NO: 5, and a CDR-L3 having the amino acid sequence of SEQ ID NO: 6; and
(iii) a second VH comprising a CDR-H1 having the amino acid sequence of SEQ ID NO: 35, a CDR-H2 having the amino acid sequence of SEQ ID NO: 36 and a CDR-H3 having the amino acid sequence of SEQ ID NO: 37; and
(c) the third and fourth polypeptides comprise a VL comprising a CDR-L1 having the amino acid sequence of SEQ ID NO: 38, a CDR-L2 having the amino acid sequence of SEQ ID NO: 39, and a CDR-L3 having the amino acid sequence of SEQ ID NO: 40,
and wherein each of the VEGF binding domains comprise a VEGF receptor component.

2. The multivalent antibody binding molecule of claim 1, wherein the LRP5 binding domain is attached to the N-terminus of the Fc domain and the FZD4 binding domain is attached to the C-terminus the Fc domain, wherein the LRP5 binding domain comprises a diabody that binds LRP5, and wherein the FZD4 binding domain comprises two scFv molecules or two Fab molecules that bind FZD4.

3-5. (canceled)

6. The multivalent antibody binding molecule of claim 1, wherein a first VEGF binding domain is attached to the C-terminus of the third polypeptide, and a second VEGF binding domain is attached to the C-terminus of the fourth polypeptide.

7. (canceled)

8. The multivalent antibody binding molecule of claim 6, wherein the first VEGF binding domain and/or the second VEGF binding domain comprises a VEGF receptor 1 (VEGFR1) extracellular Ig domain 2 and a VEGF receptor 2 (VEGFR2) extracellular Ig domain number 3.

9. The multivalent antibody binding molecule of claim 8, wherein the first VEGF binding domain and/or the second VEGF binding domain comprises an amino acid sequence at least 90% identical to a sequence selected from SEQ ID NO: 47-49.

10-13. (canceled)

14. The multivalent antibody binding molecule of claim 1, wherein:
the first VH of the first polypeptide interacts with the VL of the second polypeptide to form a domain that binds LRP5;
the first VH of the second polypeptide interacts with the VL of the first polypeptide to form a domain that binds LRP5;
the second VH of the first polypeptide interacts with the VL of the third polypeptide to form a domain that binds FZD4; and
the second VH of the second polypeptide interacts with the VL of the fourth polypeptide to form a domain that binds FZD4.

15. The multivalent antibody binding molecule of claim 1, wherein:
(a) the first polypeptide comprises:
(i) a first VH comprising an amino acid sequence that is at least 90% identical to SEQ ID NO: 13,
(ii) a VL comprising an amino acid sequence that is at least 90% identical to SEQ ID NO: 16; and
(iii) a second VH comprising an amino acid sequence that is at least 90% identical to SEQ ID NO: 41;
(b) the second polypeptide comprises:
(i) a first VH comprising an amino acid sequence that is at least 90% identical to SEQ ID NO: 15;
(ii) a VL comprising an amino acid sequence that is at least 90% identical to SEQ ID NO: 14; and
(iii) a second VH comprising an amino acid sequence that is at least 90% identical to SEQ ID NO: 41; and
(c) the third polypeptide and the fourth polypeptide comprise a VL comprising an amino acid sequence that is at least 90% identical to SEQ ID NO: 42.

16-44. (canceled)

45. The multivalent antibody binding molecule of claim 1, wherein the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 61.

46. The multivalent antibody binding molecule of claim 1, wherein the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 80.

47. The multivalent antibody binding molecule of claim 1, wherein the first polypeptide comprises the amino acid sequence of SEQ ID NO: 59, the second polypeptide comprises the amino acid of SEQ ID NO: 60, and the third polypeptide and the fourth polypeptide each comprise the amino acid sequence of SEQ ID NO: 81.

48. A pharmaceutical composition comprising the multivalent antibody binding molecule of claim 1, and a pharmaceutically acceptable carrier.

49. A method of treating an ocular disorder comprising administering to a person in need thereof a therapeutically effective amount of the multivalent antibody binding molecule of claim 1.

50. The method of claim 49, wherein the ocular disorder is selected from diabetic retinopathy, retinopathy of prematurity, Coats' disease, Familial Exudative Vitreoretinopathy (FEVR), Norrie disease, macular degeneration, diabetic macular edema, and pediatric vitreoretinopathies.

51-53. (canceled)

54. The method of claim 50, wherein the multivalent antibody binding molecule is administered by intravitreal injection.

55-64. (canceled)

65. A nucleic acid molecule encoding the multivalent antibody binding molecule of claim 1.

66. A nucleic acid molecule encoding one or more of the first polypeptide, the second polypeptide, the third polypeptide, or the fourth polypeptide of the multivalent antibody binding molecule of claim 1.

67. A vector comprising the nucleic acid molecule of claim 66.

68. A host cell comprising the nucleic acid molecule of claim 66.

69. A host cell comprising one or more vectors of claim 67.

70. A method for producing a multivalent antibody binding molecule comprising providing the host cell of claim 69, culturing the host cell in culture medium under conditions to enable expression of the multivalent antibody binding molecule, and optionally isolating the multivalent antibody binding molecule.

71. The multivalent antibody binding molecule of claim 1, wherein:
(a) the first polypeptide comprises:
(i) a first VH comprising an amino acid sequence that is at least 95% identical to SEQ ID NO: 13,
(ii) a VL comprising an amino acid sequence that is at least 95% identical to SEQ ID NO: 16; and
(iii) a second VH comprising an amino acid sequence that is at least 95% identical to SEQ ID NO: 41;
(b) the second polypeptide comprises:
(i) a first VH comprising an amino acid sequence that is at least 95% identical to SEQ ID NO: 15;
(ii) a VL comprising an amino acid sequence that is at least 95% identical to SEQ ID NO: 14; and
(iii) a second VH comprising an amino acid sequence that is at least 95% identical to SEQ ID NO: 41; and
(c) the third polypeptide and the fourth polypeptide comprise a VL comprising an amino acid sequence that is at least 95% identical to SEQ ID NO: 42, and
(d) wherein a first VEGF binding domain is attached to the C-terminus of the third polypeptide, and a second VEGF binding domain is attached to the C-terminus of the fourth polypeptide, and
(e) wherein the first VEGF binding domain and the second VEGF binding domain each comprise an amino acid sequence at least 95% identical to SEQ ID NO: 49.

72. The multivalent antibody binding molecule of claim 1, wherein:
(a) the first polypeptide comprises:
(i) a first VH comprising an amino acid sequence according to SEQ ID NO: 13,
(ii) a VL comprising an amino acid sequence according to SEQ ID NO: 16; and
(iii) a second VH comprising an amino acid sequence according to SEQ ID NO: 41;
(b) the second polypeptide comprises:
(i) a first VH comprising an amino acid sequence according to SEQ ID NO: 15;
(ii) a VL comprising an amino acid sequence according to SEQ ID NO: 14; and
(iii) a second VH comprising an amino acid sequence according to SEQ ID NO: 41; and
(c) the third polypeptide and the fourth polypeptide comprise a VL comprising an amino acid sequence according to SEQ ID NO: 42,
(d) wherein the first VEGF binding domain and the second VEGF binding domain each comprise an amino acid sequence according to SEQ ID NO: 49,
(e) wherein the first polypeptide and the second polypeptide form a heterodimer and each comprise a constant heavy chain domain 1 (CH1) comprising SEQ ID NO: 46, a constant heavy chain domain 2 (CH2) comprising an alanine at position 234 and an alanine at position 235 according to EU numbering, and a constant heavy chain domain 3 (CH3), wherein the CH3 of the first polypeptide chain comprises SEQ ID NO: 24 and the CH3 of the second polypeptide comprises SEQ ID NO: 23, and
(f) wherein the CH2 and CH3 of the first polypeptide and the CH2 and CH3 of the second polypeptide form the Fc domain.

73. The multivalent antibody binding molecule of claim 72, wherein:
(a) the VL of the first polypeptide is attached to the CH2 of the first polypeptide by a first polypeptide linker, and the VL of the second polypeptide is attached to CH2 of the second polypeptide by a second polypeptide linker,
(b) the second VH of the first polypeptide is attached to the CH3 of the first polypeptide by a third polypeptide linker, and the second VH of the second polypeptide is attached to CH3 of the second polypeptide by a fourth polypeptide linker,
(c) the first VH of the first polypeptide is linked to the VL of the first polypeptide by a fifth polypeptide linker, and the first VH of the second polypeptide is linked to the VL of the second polypeptide by a sixth polypeptide linker, and
(d) the first VEGF binding domain is attached to the second VH of the first polypeptide by a seventh polypeptide linker, and the second VEGF binding domain is attached to the second VH of the second polypeptide by an eighth polypeptide linker.

74. A pharmaceutical composition comprising the multivalent antibody binding molecule of claim 73 and a pharmaceutically acceptable carrier.

75. A method of treating an ocular disorder comprising administering to a person in need thereof a therapeutically effective amount of the multivalent antibody binding molecule of claim 73.

76. The method of claim 75, wherein the ocular disorder is selected from diabetic retinopathy, retinopathy of prematurity, Coats' disease, Familial Exudative Vitreoretinopathy (FEVR), Norrie disease, macular degeneration, diabetic macular edema, and pediatric vitreoretinopathies.

77. The method of claim 76, wherein the multivalent antibody binding molecule is administered by intravitreal injection.

78. A nucleic acid molecule encoding the multivalent antibody binding molecule of claim 73.

79. A nucleic acid molecule encoding one or more of the first polypeptide, the second polypeptide, the third polypeptide, or the fourth polypeptide of the multivalent antibody binding molecule of claim 73.

80. A vector comprising the nucleic acid molecule of claim 79.

81. A host cell comprising the nucleic acid molecule of claim 79.

82. A host cell comprising one or more vectors of claim 80.

83. A method for producing a multivalent antibody binding molecule comprising providing the host cell of claim 82, culturing the host cell in culture medium under conditions to enable expression of the multivalent antibody binding molecule, and optionally isolating the multivalent antibody binding molecule.
